(19) United States
(12) Patent Application Publication
(10) Pub. No.: US 2021/0379044 A1
(43) Pub. Date: Dec. 9, 2021

(54) NON PEPTIDIC HETEROBIVALENT MOLECULES FOR TREATING INFLAMMATORY DISEASES

(71) Applicant: GLAXOSMITHKLINE INTELLECTUAL PROPERTY DEVELOPMENT LIMITED, Brentford (GB)

(72) Inventors: James BAILEY, Stevenage (GB); Yao CHEN, Collegeville, PA (US); Mark HURLE, Collegeville, PA (US); Craig LEACH, Collegeville, PA (US); Brandon TURUNEN, Collegeville, PA (US)

(73) Assignee: GLAXOSMITHKLINE INTELLECTUAL PROPERTY DEVELOPMENT LIMITED

(21) Appl. No.: 17/412,070

(22) Filed: Aug. 25, 2021

Related U.S. Application Data
(63) Continuation of application No. 16/478,551, filed on Jul. 17, 2019.
(60) Provisional application No. 62/446,959, filed on Jan. 17, 2017.

(51) Int. Cl. A61K 31/454; A61K 31/4375; A61K 31/438; A61K 31/496; A61K 31/499; A61K 31/506; A61K 31/5375; A61K 31/5377

(52) U.S. Cl. CPC A61K 31/454; A61K 31/4375; A61K 31/438; A61K 31/5377; A61K 31/499; A61K 31/506; A61K 31/5375; A61K 31/496

(57) ABSTRACT

The present invention relates to non peptidic, heterobivalent molecules (HBM) that are able to simultaneously bind a surface target protein as well as an endogenous or exogenous human antibody protein and induce immune effector function. More specifically, the present invention relates to agents capable of binding to a chemokine receptor and inducing the depletion of chemokine receptor positive subsets of pathogenic cells in a subject for use in the treatment and/or prevention of cancer, inflammatory, autoimmune and allergic disease.

### FIELD OF INVENTION

The present invention relates to non peptidic, heterobivalent molecules (HBM) that are able to simultaneously bind a surface target protein as well as an endogenous or exogenous human antibody protein and induce immune effector function. More specifically, the present invention relates to agents capable of binding to a chemokine receptor and inducing the depletion of chemokine receptor positive subsets of pathogenic cells in a subject for use in the treatment and/or prevention of cancer, inflammatory, autoimmune and allergic disease.

### BACKGROUND OF THE INVENTION

Chemokine ligand/receptors play key roles in a range of inflammatory, allergic and autoimmune diseases as well tumor initiation, growth and metastasis. At the sites of inflammation cells release a defined set of inflammatory chemokines that are responsible for the recruitment of activated pathological leukocytes. Recruited immune cells synthesize and release a host of inflammatory mediators and are responsible for the maintenance and escalation of inflammatory responses, secondary tissue damage, and the promotion of autoimmunity, fibrosis and tissue remodelling. Predominant leukocyte subtype populations with defined up regulation of inflammatory chemokine receptors are associated with specific diseases (Table 1).

Examples of this include CCR1 expression on Myeloid Derived Suppressor Cells (MDSCs) in the tumor microenvironment and CCR2 positive monocytes and macrophages in human glomerulonephritides and nephropathies. Also, CCR3 positive eosinophils and Th2 cells are associated with allergic asthma and rhinitis. Many cancers over express one or more chemokine receptors. As an example, CCR4 has been 52 cells (Tregs) in the tumor microenvironment.

TABLE 1
Chemokine receptor, disease association and related cell type

| Chemokine Receptor | Associated Disease | Cell Type |
|---|---|---|
| CCR1 | Cancer, RSV, RA | MDSCs, Cancer cells |
| CCR2 | MS, Cancer, Lupus, RA | MDSC’s, TAMs, |
| | | TH1 and TH17 |
| CCR3 | Allergies and Asthma | Eosinophils, TH2 cells |
| CCR4 | Asthma and Cancer | Tregs, Cancer cells |
| CCR5 | HIV and MS | Thi and Tregs |
| CCR6 | Asthma, RA, MS, Cancer | Th17 and Cancer cells |
| CCR9 | IBD and Colitis | T-Cell subsets |

Abbreviations: RSV, Respiratory Syncytial Virus; RA, Rheumatoid Arthritis; MDSCs, Myeloid Derived Suppressor Cells; MS, Multiple Sclerosis; TAMs, Tumor Associated Macrophages; TH1, T Helper 1 cells; TH17, T Helper 17 cells; Tregs, T regulatory cells; HIV, Human Immunodeficiency Virus; IBD, Inflammatory Bowel Disease.

### SUMMARY OF THE INVENTION

In one aspect of the invention there is provided a Heterobivalent molecule (HBM) wherein HBM comprise a moiety binding to a CCR receptor on a cell and a moiety binding to endogenous or exogenous antibodies.

In further aspect, the HBM further comprises a linker.

In further aspect, the HBM has chemical structure represented by P-Q-R, in which P represents a CCR binding moiety, Q represents a chemical linker, and R represents a moiety binding to the endogenous or exogenous antibody.

In further aspect, the moiety binding to endogenous or exogenous antibody is selected from the group consisting of DNP, fluorescein, cotinine and biotin, or derivative thereof.

In one aspect of the invention there is provided a Heterobivalent molecule (HBM) wherein HBM comprise a moiety binding to a CCR receptor on a cell and a moiety binding to endogenous or exogenous antibodies for use in therapy.

In a further embodiment, the present invention provides a pharmaceutical composition comprising HBM and one or more pharmaceutical acceptable excipients, diluents, and/or carriers.

In a further aspect of the present invention, there is provided a method of treating diseases and conditions mediated by the CCR receptor in a subject comprising administering a therapeutically effective amount of a a Heterobivalent molecule (HBM) wherein HBM comprise a moiety binding to a CCR receptor on a cell and a moiety binding to endogenous or exogenous antibodies.

In a further aspect of the present invention, there is provided the use of a Heterobivalent molecule (HBM) wherein HBM comprise a moiety binding to a CCR receptor on a cell and a moiety binding to endogenous or exogenous antibodies or a pharmaceutically acceptable salt thereof in the manufacture of a medicament for use in treating diseases and conditions mediated by the CCR receptor.

In one aspect of the invention, the present invention relates to a method of destroying CCR-positive cells in a human using a Heterobivalent Molecule (HBM), wherein HBM comprise a moiety binding to a CCR receptor on the cell and a moiety binding to endogenous or exogenous antibodies.

In further aspect, the cell destruction is mediated through ADCC, ADCP and/or CDC.

In further aspect, the cells being destroyed are cancer cells and/or pathogenic immune cells.

In further aspect, the cancer cells and/or pathogenic immune cells express one or more CCR receptors selected from the group of CCR1, CCR2, CCR3, and CCR5.

In another aspect, the present invention relates to preventing or treating cancers, inflammatory disease, autoimmune disease, or allergic disease in a human patient comprising administering a therapeutic effective amount of a HBM to the human patient.

Yet in another aspect, HBM can be given parenterally or orally.

In further embodiment, the present invention provides for the use of HBM for the manufacture of a medicament for the treatment or prevention of cancers, inflammatory disease, autoimmune disease, or allergic disease in a human patient.

In further embodiment, the present invention relates to a pharmaceutical composition for treating or preventing cancers, inflammatory disease, autoimmune disease, or allergic disease in a human patient comprising HBM.

In a further aspect of the present invention, an exogenous monoclonal antibody, which binds to a portion of the HBM, is administered to the patient. The combination of the HBM and exogenous monoclonal antibody comprise medicament for use in treating diseases and conditions mediated by the CCR receptor.

### DESCRIPTION OF THE INVENTION

The present invention relates to a small molecule agent capable of binding a member of the chemokine receptor family and inducing the depletion of CCR-positive cells in a subject for use in the treatment and/or prevention of a disease. The present invention further relates to a method of treating and/or preventing an immune driven disease via selective depletion of pathogenic immune cells by administering a pharmaceutically effective amount of an agent or combination of agents capable of binding to a CCR and inducing the depletion of the CCR-positive cells. Furthermore, the present invention relates to a pharmaceutical composition comprising the agent of the invention which will be termed “heterobivalent molecules” (HBM). In another aspect, the invention may include passive immunization with a monoclonal antibody which binds the HBM and induces immune effector function in the presence of CCR-positive cells.

Antibody based therapeutics (ABTs) suffer from poor bioavailability, high cost, thermal instability, and difficult manufacturing due to their size, complexity and peptide based structures. HBMs are a class of immunotherapeutics that promises the affordability, stability and oral dosing of small molecules, the selectivity and immune control of a therapeutic antibody and the lasting immunity of a vaccine. These bifunctional synthetic agents are designed such that one terminus interacts with a disease-relevant, extracellular biomolecular target (for example CCRs), while the other binds endogenous pools of specific antibody proteins (or effector cell directly). This complex directs immune surveillance to target expressing tissue/cells and disrupts signaling in the same fashion as a biological based monoclonal antibody. This mechanism may include antibody dependent cellular cytotoxicity (ADCC), antibody dependent cellular phagocytosis (ADCP), complement dependant cytotocity (CDC) or ligand mediated neutralization. The same Fc receptor expressing immune cells that initiate destruction of the ARM (antibody retargeting molecule, herein also referred to as HBM)/antibody tagged cells also participate in presentation of endogenous antigens for the potential for long term cellular immunity.

Many preclinical examples exist demonstrating one can directly target a tumor specific antigen, a virus or bacteria. The Spiegel group at Yale has targeted prostate specific membrane antigen (PSMA), and recruited dinitrophenyl (DNP) antibodies, inhibiting tumor growth and prolonging survival in a mouse in-vivo model. These researchers also developed a HIV-targeted ARM using reported fusion inhibitor BMS-378806 coupled to the DNP hapten. This chemical tool induced destruction of HIV infected cells as well as exhibited competitive binding with CD4, blocking entry of the HIV-1 virus. George M. Whitesides directed phagocytosis of various Gram-positive bacteria (S. epidermidis, S. pneumoniae, and S. aureus) utilizing vancomycin as a targeting agent and fluorescein as the hapten. Also attempts are being made to effect the CCR4+ immune cell depletion with an ADCC enhanced mAb to evoke anti-tumor activities, no one to date has used HBM to rebalance the immune system to allow a patient's own immune response to treat diseases, such as cancer, inflammatory, autoimmune and allergic diseases. (D. Sugiyama et al., PNAS, Vol 110, no. 44, Oct. 29, 2013) Using biological based therapies (PDL1, CTLA4, etc) rebalancing the immune system has had profound clinical effects in patients. As used herein:

“Pathogenic immune cells” includes a particular immune cell subset that causes or is capable of causing disease. These cellular subsets are resident cells or are recruited to particular locations and secrete cytokines, chemokines and other mediators and contribute to the persistence and progression of disease such as cancer in the case of a tumor microenvironment or chronic inflammation of the lung in the case of asthma (there are many other examples). Examples of pathogenic immune cells are listed in “Cell Type” column of Table 1.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

Compounds which targets chemokine receptors are very well studied and described. (See for example: M. Wijtmans et al., Drug Discovery Today; Technologies, Vol 9, No 4, 2012, p e229; M. Allegretti et al., Immunology Letters 145 (2012) p 68-78) A person skilled the art can select the known molecules to be used as a moiety which bind to CCRs in HBM. Further, there are many examples in the art disclosing molecules which binds to antibodies, such as dinitrophenyl (DNP), fluorescein, cotinine and biotin, and derivatives thereof. These known molecules can used as a part of HBM. To illustrate the principle that a skilled person can construct HBM from known compounds, a number of actual HBM examples are provided in Section 1.1 Compounds section only as a way of illustration, and by no means limit the invention in any manner.

The HBM has chemical structure represented by a structure of formula I,

P-Q-R I

in which P represents a CCR binding moiety, Q represents a chemical linker, and R represents a moiety binding to the endogenous or exogenous antibody.

Example of R can be represented as, but not limited to a radical of the formula:

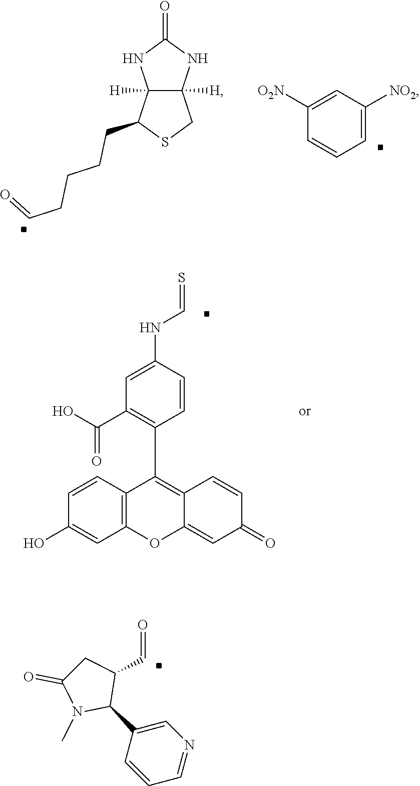

Further Q can be represented as, but not limited to, a radical of the formula:

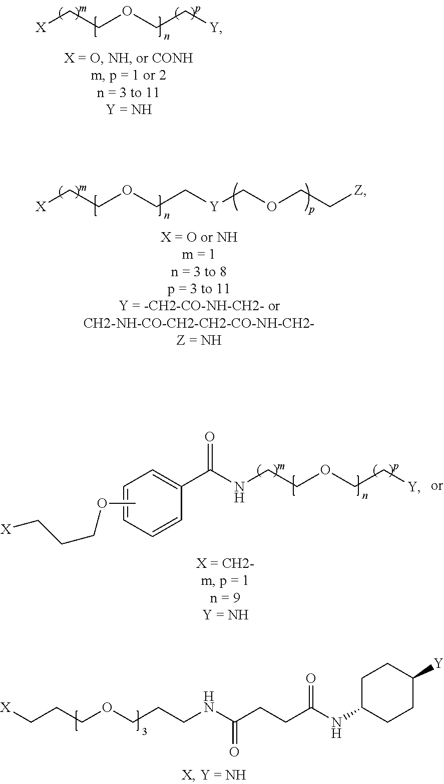

### SPECIFIC EMBODIMENTS

The compounds described herein have been characterized in an ADCC reporter assay (see Section 1.2 ADCC reporter for protocol). This assay format can be used to demonstrate that the heterobivalent small molecule (HBM) is able to simultaneously bind the cell surface target as well as the antibody protein. Furthermore, the assay also confirms that this formed complex can engage and activate the Fc receptors on immune cells. Lastly, the assay provides both potency (EC50) and efficacy (signal to background ratio) for each compound in a dose dependant manner.

The HBMs targeting receptors CCR1, CCR2, CCR3 and CCR5 have demonstrated the ability to simultaneously bind the surface target protein and specific antibody proteins. Once formed, this ternary complex is able to induce cell to cell contact with the target cell and the effector cell. We detail in Table 2 below these results and demonstrating a range of potencies and efficacies. For example, compound 18 which targets CCR3, exhibits potency in the nanomolar range and a robust efficacy signal providing confidence this would be an effective cell depleting agent.

TABLE 2
Antibody Dependent Cellular Cytotoxicity Reporter Assay for
HBMs targeting a variety of chemokine receptors

| Chemokine | Compound | ADCC | ADCC |
|---|---|---|---|
| Receptor | Number | potency^{a} | efficacy^{b} |
| CCR1 | 1 | + | +++^{d} |
| CCR1 | 2 | +/− | ++^{d} |
| CCR1 | 3 | + | ++^{d} |
| CCR1 | 4 | +/− | ++^{d} |
| CCR1 | 5 | +/− | +^{d} |
| CCR2 | 6 | +++ | +++^{e} |
| CCR2 | 7 | +++ | +++^{e} |
| CCR2 | 8 | + | +++^{e} |
| CCR2 | 9 | +++ | +++^{e} |
| CCR2 | 10 | ++ | +++^{e} |
| CCR2 | 11 | + | +++^{e} |
| CCR2 | 12 | +++ | +++^{e} |
| CCR2 | 13 | ++ | +++^{e} |
| CCR2 | 14 | +/− | ++^{e} |
| CCR2 | 15 | ++ | +++^{e} |
| CCR2 | 16 | +++ | ++^{e} |
| CCR2 | 17 | +++ | +++^{e} |
| CCR2 | 18 | +++ | +++^{e} |
| CCR2 | 19 | +++ | +++^{e} |
| CCR3 | 20 | +++ | +++^{f} |
| CCR3 | 21 | +++ | +++^{f} |
| CCR3 | 22 | ++ | +++^{f} |
| CCR3 | 23 | +++ | +++^{f} |
| CCR3 | 25 | ++ | +++^{f} |
| CCR5 | 20 | +++ | +++^{g} |

^{a}Antibody Dependent Cellular Cytotoxity potency is defined by the amount of compound required to achieve 50% of maximal response (EC50)
^{b}Antibody Dependent Cellular Cytotoxity effcacy is defined quantitatively by the effectivness of the compound to induce complex formation between target cell, antibody and effector cell and is measured by ratio of the signal to background S/B
^{c}The range of potencies is broken down in the scale, pEC50 ≥ 9 = +++, 9 > pEC50 ≥ 8 = ++ 8 > pEC50 ≥ 7, 7 > pEC50 ≥ 6 = +/−
^{d}The efficacy range is described as a maximum signal: background >11 = +++, between 9-11 = ++, between 6-8 = +
^{e}Maximum Signal: Background >10 = +++, >5 = ++
^{f}Maximum S: B >20 = +++,
^{g}Maximum Signal: Background >70 = +++

### Section 1.1: Compounds

### CCR1

### N1-((R)-1-(4-(4-chlorophenyl)piperidin-1-yl)-3-methyl-1-oxobutan-2-yl)-N3-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)isophthalamide

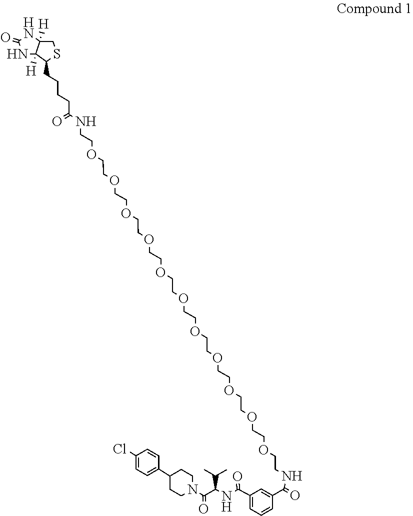 [SMILES: CC(C)[C@@H](NC(=O)c1cccc(C(=O)NCCOCCOCCOCCOCCOCCOCCOCCOCCOCCOCCOCCNC(=O)CCCC[C@@H]2SC[C@@H]3NC(=O)N[C@@H]32)c1)C(=O)N1CCC(c2ccc(Cl)cc2)CC1]

### Step 1

### (R)-Tert-butyl (1-(4-(4-chlorophenyl)piperidin-1-yl)-3-methyl-1-oxobutan-2-yl)carbamate

Under a nitrogen atmosphere, a solution of (R)-2-((tert-butoxycarbonyl)amino)-3-methylbutanoic acid (5.55 g, 25.6 mmol) and HATU (10.69 g, 28.1 mmol) and HOBt (0.391 g, 2.56 mmol) dissolved in dichloromethane (DCM) (85 ml) was prepared at room temperature in a 100 mL. The mixture was treated with DIEA (13.39 ml, 77 mmol) and appeared to be some what homogeneous. Afterwards, to the mixture was added 4-(4-chlorophenyl)piperidine (5.0 g, 25.6 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)]((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1:1 mixture of saturated sodium bicarbonate and water and brine. The organic layer was passed through a phase separator and concentrated to give the crude product, which was purified by flash chromatography using a Biotage Isolera. A 120 g silica column was used along with a gradient of 0%(3cv)-0-40%(20CV) 40%(4CV) ehtyl acetate/hexanes at a flow rate of 100 mL/min. All fractions containing the desired product were combined and concentrated to give the desired product, (R)-tert-butyl (1-(4-(4-chlorophenyl)piperidin-1-yl)-3-methyl-1-oxobutan-2-yl)carbamate (7.65 g, 18.40 mmol, 72.0% yield), as an colorless oil. Purity was judged >95% and the analytical characterization data of the final material was consistent with the assigned structure. All of this material was carried on as a synthetic intermediate. MS (ES) m/e 354.1 [M+H]+, rt=0.92 mins.

### Step 2

### (R)-2-Amino-1-(4-(4-chlorophenyl)piperidin-1-yl)-3-methylbutan-1-one, Hydrochloride

Under a nitrogen atmosphere, a solution of (R)-tert-butyl (1-(4-(4-chlorophenyl)piperidin-1-yl)-3-methyl-1-oxobutan-2-yl)carbamate (7.6 g, 19.24 mmol) dissolved in 1,4-dioxane (64.1 ml) was prepared at room temperature in a 250 mL round bottom flask. The mixture was treated with 4M HCl in dioxane (60.1 ml, 241 mmol) and appeared to be some what homogeneous. Afterwards, to the mixture was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was concentrated to give the crude product, (R)-2-amino-1-(4-(4-chlorophenyl)piperidin-1-yl)-3-methylbutan-1-one, Hydrochloride (6.98 mg, 0.020 mmol, 0.104% yield), as an colorless oil. Purity was judged >95% and the analytical characterization data of the final material was consistent with the assigned structure. All of the isolated material was carried on as a synthetic intermediate. MS (ES) m/e 295 [M+H]+, rt=0.83 mins.

### Step 3

### (R)-Methyl-2-((1-(4-(4-chlorophenyl)piperidin-1-yl)-3-methyl-1-oxobutan-2-yl)carbamoyl)benzoate

Under a nitrogen atmosphere, a solution of 2-(methoxycarbonyl)benzoic acid (272 mg, 1.509 mmol) and HATU (603 mg, 1.585 mmol) and HOBt (23.11 mg, 0.151 mmol) dissolved in dichloromethane (DCM) (5031 μl) was prepared at room temperature in a small microwave vial. The mixture was treated with DIEA (791 μl, 4.53 mmol) and appeared to be some what homogeneous. Afterwards, to the mixture was added (R)-2-amino-1-(4-(4-chlorophenyl)piperidin-1-yl)-3-methylbutan-1-one, Hydrochloride (500 mg, 1.509 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1:1 mixture of saturated sodium bicarbonate and water and brine. The organic layer was passed through a phase separator and concentrated to give the crude product, which was purified by flash chromatography using a Biotage Isolera. A 120 g silica column was used along with a gradient of 0%(3cv)-0-40%(20CV) 40%(4CV) ethyl acetate/hexanes at a flow rate of 100 mL/min. All fractions containing the desired product were combined and concentrated to give the desired product, (N43755-5-101) (R)-methyl 2-((1-(4-(4-chlorophenyl)piperidin-1-yl)-3-methyl-1-oxobutan-2-yl)carbamoyl)benzoate (553 mg, 1.210 mmol, 80% yield), as an off-white solid. Purity was judged >95% and the analytical characterization data of the final material was consistent with the assigned structure. All of this material was carried on as a synthetic intermediate. MS (ES) m/e 457 [M+H]+, rt=1.19 mins.

### Step 4

### (R)-3-((1-(4-(4-chlorophenyl)piperidin-1-yl)-3-methyl-1-oxobutan-2-yl)carbamoyl)benzoic acid

(R)-methyl 3-((1-(4-(4-chlorophenyl)piperidin-1-yl)-3-methyl-1-oxobutan-2-yl)carbamoyl)benzoate (534.2 mg, 1.169 mmol) was taken up in a 2:1:1 mixture of MeOH:THF:H2O, 1 mL total volume. Afterwards, KOH (1998 μl, 23.38 mmol), was added at room temperature and the mixture was allowed to stir at 20° C. for 20 hr. The progress was checked by Open Access LCMS [Shimadzu 10A PE (LC) coupled with Sciex Single Quadrupole 150EX (MS) and Sedere Sedex 75C (ELS)]((Thermo Hypersil Gold C18, 20×2.1 mm, 1.9 u particle diam.), 1.6 mL/min, gradient from 5-92% CH3CN (0.02% TFA)/H2O (0.02% TFA)). After 20 hours, the spectra confirmed that the reaction progressed fairly cleanly to give the desired product. As a result, more catalyst was added and the reaction was allowed to continue for another 17 hours. The mixture was concentrated under vacuum to give a crude residue. The residue was dissolved in a minimum amount of water and treated with 6N HCl to lower the pH to 5. A white solid precipitated out of solution and was collected by vacuum filtration and rinsed with water several times. After drying in a vacuum oven, the crude product, (R)-3-((1-(4-(4-chlorophenyl)piperidin-1-yl)-3-methyl-1-oxobutan-2-yl)carbamoyl)benzoic acid (530 mg, 1.077 mmol, 92% yield), was recovered as an off-white presumed amorphous solid after drying in a vacuum oven overnight. Purity was judged >95% and the analytical characterization data of the final material was consistent with the assigned structure. This material was carried forward as a synthetic intermediate. MS (ES) m/e 710.5 [M+H]+, rt=0.89 mins.

### Step 5

### N1-((R)-1-(4-(4-chlorophenyl)piperidin-1-yl)-3-methyl-1-oxobutan-2-yl)-N3-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)isophthalamide

Under a nitrogen atmosphere, a solution of (R)-3-((1-(4-(4-chlorophenyl)piperidin-1-yl)-3-methyl-1-oxobutan-2-yl)carbamoyl)benzoic acid (40 mg, 0.090 mmol), HATU (34.7 mg, 0.091 mmol), and HOBt (1.383 mg, 9.03 μmol) dissolved in N,N-dimethylformamide (DMF) (903 μl) was prepared at room temperature in a small 4 mL vial. The mixture was treated with DIEA (47.3 μl, 0.271 mmol) and appeared to be some what homogeneous. Afterwards, to the mixture was added N-(35-amino-3,6,9,12,15,18,21,24,27,30,33-undecaoxapentatriacontyl)-5-((3aS,4S,6aR)-2-oxohexahydro-1H-thieno[3,4-d]imidazol-4-yl)pentanamide (69.6 mg, 0.090 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was purified by reverse phase HPLC [40-70% acetonitrile:water (0.1% NH4OH modifier), C18 50×30 mm GEMINI column, 47 mL/min]. All fractions containing the desired product were combined and concentrated to give the desired product, N1-((R)-1-(4-(4-chlorophenyl)piperidin-1-yl)-3-methyl-1-oxobutan-2-yl)-N3-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)isophthalamide (34.6 mg, 0.027 mmol, 30.4% yield), as a yellow oil after lyopholizing. Purity was judged >95% and the analytical characterization data of the final material was consistent with the assigned structure. All of this material was submitted for biological testing and storage.

MS (ES) m/e 1195.9 [M+H]+, rt=1.1 mins. ¹H NMR (400 MHz, DMSO-d₆) □ ppm 8.54-8.68 (m, 2H) 8.34 (d, J=11.00 Hz, 1H) 8.00 (dd, J=17.85, 7.09 Hz, 2H) 7.81 (t, J=5.50 Hz, 1H) 7.55 (t, J=7.70 Hz, 1H) 7.16-7.42 (m, 4H) 6.29-6.45 (m, 2H) 4.80 (t, J=8.56 Hz, 1H) 4.58 (d, J=12.72 Hz, 1H) 4.21-4.42 (m, 2H) 4.12 (dd, J=7.46, 4.52 Hz, 1H) 3.44-3.60 (m, 46H) 3.02-3.24 (m, 4H) 2.81 (dd, J=12.35, 5.01 Hz, 2H) 2.54-2.75 (m, 2H) 2.21 (dt, J=14.24, 7.18 Hz, 1H) 2.06 (t, J=7.34 Hz, 2H) 1.69-1.95 (m, 2H) 1.21-1.66 (m, 8H) 0.86-1.02 (m, 6H)

### N1-((R)-1-(4-(4-chlorophenyl)piperidin-1-yl)-3-methyl-1-oxobutan-2-yl)-N3-(16-oxo-20-((3aS,4S,6aR)-2-oxohexahydro-1H-thieno[3,4-d]imidazol-4-yl)-3,6,9,12-tetraoxa-15-azaicosyl)i sophthalamide

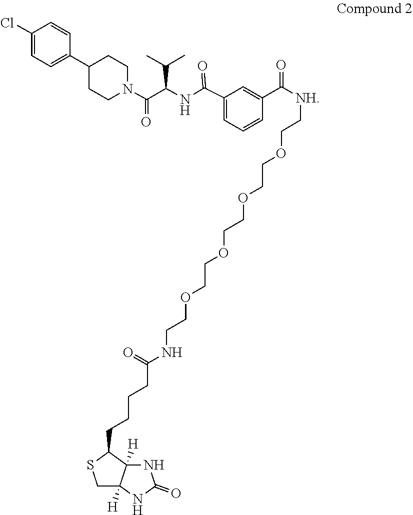 [SMILES: CC(C)[C@@H](NC(=O)c1cccc(C(=O)NCCOCCOCCOCCOCCNC(=O)CCCC[C@@H]2SC[C@@H]3NC(=O)N[C@@H]32)c1)C(=O)N1CCC(c2ccc(Cl)cc2)CC1]

### Repeated Steps 1-4 Described for Compound 1

### Step 1 (Compound 2)

### N1-((R)-1-(4-(4-chlorophenyl)piperidin-1-yl)-3-methyl-1-oxobutan-2-yl)-N3-(16-oxo-20-((3aS,4S,6aR)-2-oxohexahydro-1H-thieno[3,4-d]imidazol-4-yl)-3,6,9,12-tetraoxa-15-azaicosyl)isophthalamide

Under a nitrogen atmosphere, a solution of (R)-3-((1-(4-(4-chlorophenyl)piperidin-1-yl)-3-methyl-1-oxobutan-2-yl)carbamoyl)benzoic acid (40 mg, 0.090 mmol), HATU (34.7 mg, 0.091 mmol), and HOBt (1.383 mg, 9.03 μmol) dissolved in N,N-dimethylformamide (DMF) (903 μl) was prepared at room temperature in a small 4 mL vial. The mixture was treated with DIEA (47.3 μl, 0.271 mmol) and appeared to be some what homogeneous. Afterwards, to the mixture was added N-(14-amino-3,6,9,12-tetraoxatetradecyl)-5-((3aS,4S,6aR)-2-oxohexahydro-1H-thieno[3,4-d]imidazol-4-yl)pentanamide (41.8 mg, 0.090 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was purified by reverse phase HPLC [40-70% acetonitrile:water (0.1% NH4OH modifier), C18 50×30 mm GEMINI column, 47 mL/min]. All fractions containing the desired product were combined and concentrated to give the desired product, (N43755-41-101) N1-((R)-1-(4-(4-chlorophenyl)piperidin-1-yl)-3-methyl-1-oxobutan-2-yl)-N3-(16-oxo-20-((3aS,4S,6aR)-2-oxohexahydro-1H-thieno[3,4-d]imidazol-4-yl)-3,6,9,12-tetraoxa-15-azaicosyl)isophthalamide (40.7 mg, 0.044 mmol, 48.2% yield), as a yellow oil after lyopholizing. Purity was judged >95% and the analytical characterization data of the final material was consistent with the assigned structure. All of this material was submitted for biological testing and storage. MS (ES) m/e 887.5 [M+H]+, rt=1.05 mins. ¹H NMR (400 MHz, DMSO-d₆) □ ppm 8.54-8.67 (m, 2H) 8.34 (d, J=10.76 Hz, 1H) 8.00 (dd, J=17.97, 6.97 Hz, 2H) 7.81 (t, J=5.50 Hz, 1H) 7.55 (t, J=7.70 Hz, 1H) 7.17-7.39 (m, 4H) 6.40 (br. s., 1H) 4.80 (t, J=8.56 Hz, 1H) 4.58 (d, J=13.69 Hz, 1H) 4.23-4.40 (m, 3H) 4.12 (dd, J=7.58, 4.40 Hz, 2H) 3.42-3.59 (m, 14H) 3.37 (t, J=5.87 Hz, 2H) 3.05-3.23 (m, 4H) 2.81 (dd, J=12.47, 5.14 Hz, 2H) 2.54-2.75 (m, 3H) 2.14-2.26 (m, 1H) 2.06 (t, J=7.34 Hz, 2H) 1.71-1.94 (m, 2H) 1.22-1.65 (m, 8H) 0.89-1.01 (m, 6H)

### N1-((R)-1-(4-(4-chlorophenyl)piperidin-1-yl)-3-methyl-1-oxobutan-2-yl)-N3-(25-oxo-29-((3aS,4S,6aR)-2-oxohexahydro-1H-thieno[3,4-d]imidazol-4-yl)-3,6,9,12,15,18,21-heptaoxa-24-azanonacosyl)isophthalamide

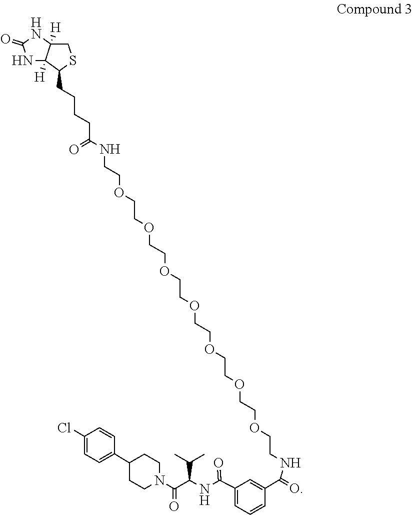 [SMILES: CC(C)[C@@H](NC(=O)c1cccc(C(=O)NCCOCCOCCOCCOCCOCCOCCOCCNC(=O)CCCC[C@@H]2SC[C@@H]3NC(=O)N[C@@H]32)c1)C(=O)N1CCC(c2ccc(Cl)cc2)CC1]

### Repeated Steps 1-4 Described for Compound 1

### Step 1 (Compound 3)

### N1-((R)-1-(4-(4-chlorophenyl)piperidin-1-yl)-3-methyl-1-oxobutan-2-yl)-N3-(25-oxo-29-((3aS,4S,6aR)-2-oxohexahydro-1H-thieno[3,4-d]imidazol-4-yl)-3,6,9,12,15,18,21-heptaoxa-24-azanonacosyl)isophthalamide

Under a nitrogen atmosphere, a solution of (R)-3-((1-(4-(4-chlorophenyl)piperidin-1-yl)-3-methyl-1-oxobutan-2-yl)carbamoyl)benzoic acid (40 mg, 0.090 mmol), HATU (34.7 mg, 0.091 mmol), and HOBt (1.383 mg, 9.03 μmol) dissolved in N,N-dimethylformamide (DMF) (903 μl) was prepared at room temperature in a small 4 mL vial. The mixture was treated with DIEA (47.3 μl, 0.271 mmol) and appeared to be some what homogeneous. Afterwards, to the mixture was added N-(23-amino-3,6,9,12,15,18,21-heptaoxatricosyl)-5-((3aS,4S,6aR)-2-oxohexahydro-1H-thieno[3,4-d]imidazol-4-yl)pentanamide (53.7 mg, 0.090 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 □m particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was purified by reverse phase HPLC [40-70% acetonitrile:water (0.1% NH4OH modifier), C18 50×30 mm GEMINI column, 47 mL/min]. All fractions containing the desired product were combined and concentrated to give the desired product, (N43755-40-101) N1-((R)-1-(4-(4-chlorophenyl)piperidin-1-yl)-3-methyl-1-oxobutan-2-yl)-N3-(25-oxo-29-((3aS,4S,6aR)-2-oxohexahydro-1H-thieno[3,4-d]imidazol-4-yl)-3,6,9,12,15,18,21-heptaoxa-24-azanonacosyl)isophthalamide (42 mg, 0.039 mmol, 43.3% yield), as a yellow oil after lyopholizing. Purity was judged >95% and the analytical characterization data of the final material was consistent with the assigned structure (1HNMR:N43755-40-101; LCMS:N43755-40-101). All of this material was submitted for biological testing and storage.

MS (ES) m/e 1019.7 [M+H]+, rt=1.09 mins. ¹H NMR (400 MHz, DMSO-d₆) □ ppm 8.54-8.66 (m, 2H) 8.34 (d, J=11.00 Hz, 1H) 7.94-8.05 (m, 2H) 7.81 (t, J=5.50 Hz, 1H) 7.55 (t, J=7.70 Hz, 1H) 7.15-7.39 (m, 5H) 6.40 (br. s., 2H) 4.80 (t, J=8.44 Hz, 1H) 4.58 (d, J=13.94 Hz, 1H) 4.23-4.40 (m, 3H) 4.12 (dd, J=7.58, 4.40 Hz, 2H) 3.42-3.59 (m, 24H) 3.38 (t, J=5.99 Hz, 2H) 3.04-3.23 (m, 4H) 2.81 (dd, J=12.47, 5.14 Hz, 2H) 2.53-2.75 (m, 3H) 2.13-2.28 (m, 1H) 2.06 (t, J=7.34 Hz, 2H) 1.71-1.94 (m, 2H) 1.21-1.67 (m, 8H) 0.88-1.03 (m, 6H)

### N1-((R)-1-(4-(4-chlorophenyl)piperidin-1-yl)-3-methyl-1-oxobutan-2-yl)-N3-(15,53-dioxo-57-((3aS,4S,6aR)-2-oxohexahydro-1H-thieno[3,4-d]imidazol-4-yl)-3,6,9,12,19,22,25,28,31,34, 37,40,43,46,49-pentadecaoxa-16,52-diazaheptapentacontyl)isophthalamide

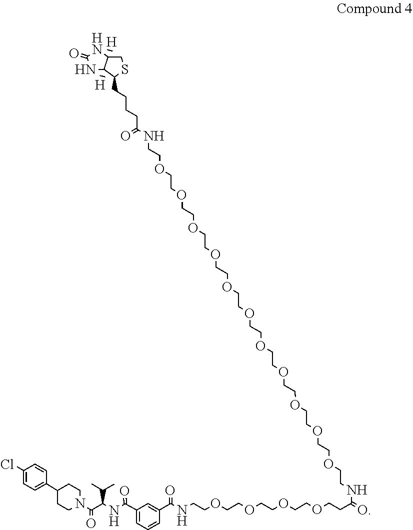 [SMILES: CC(C)[C@@H](NC(=O)c1cccc(C(=O)NCCOCCOCCOCCOCCC(=O)NCCOCCOCCOCCOCCOCCOCCOCCOCCOCCOCCOCCNC(=O)CCCC[C@@H]2SC[C@@H]3NC(=O)N[C@@H]32)c1)C(=O)N1CCC(c2ccc(Cl)cc2)CC1]

### Repeated Steps 1-4 Described for Compound 1

### Step 1 (Compound 4)

### 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-diazaheptapentacontyl)-3,6,9,12-tetraoxapentadecan-15-amide

Under a nitrogen atmosphere, a solution of 2,2-dimethyl-4-oxo-3,8,11,14,17-pentaoxa-5-azaicosan-20-oic acid (31.6 mg, 0.086 mmol), HATU (33.2 mg, 0.087 mmol), and HOBt (1.324 mg, 8.65 μmol) was prepared in dichloromethane (DCM) (865 μl) at ambient temperature. The mixture was homogeneous. Afterwards, DIEA (45.3 μl, 0.259 mmol) was delivered. After 10 mins the mixture was treated with N-(35-amino-3,6,9,12,15,18,21,24,27,30,33-undecaoxapentatriacontyl)-5-((3aS,4S,6aR)-2-oxohexahydro-1H-thieno[3,4-d]imidazol-4-yl)pentanamide (66.7 mg, 0.086 mmol) and was allowed to stir for 18 hours. The progress was checked by Open Access LCMS [Shimadzu 10A PE (LC) coupled with Sciex Single Quadrupole 150EX (MS) and Sedere Sedex 75C (ELS)]((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1:1 mixture of saturated sodium bicarbonate and water and brine. The organic layer was passed through a phase separator and concentrated to give the desired product, tert-butyl (15,53-dioxo-57-((3aS,4S,6aR)-2-oxohexahydro-1H-thieno[3,4-d]imidazol-4-yl)-3,6,9,12,19,22,25,28,31,34,37,40,43,46,49-pentadecaoxa-16,52-diazaheptapentacontyl)carbamate (102 mg, 0.082 mmol, 95% yield), as a clear colorless oil. Purity was judged >90% and the analytical characterization data of the final material was consistent with the assigned structure. MS (ES) m/e 1119 [M+H]+, rt=0.83 mins.

Again, under a nitrogen atmosphere, a solution of tert-butyl (15,53-dioxo-57-((3aS,4S,6aR)-2-oxohexahydro-1H-thieno[3,4-d]imidazol-4-yl)-3,6,9,12,19,22,25,28,31,34,37,40,43,46,49-pentadecaoxa-16,52-diazaheptapentacontyl)carbamate (102 mg, 0.082 mmol) was prepared in 1 mL 1,4 dioxane at ambient temperature. The mixture was homogeneous. Afterwards, 4M solution of HCl in dioxane (216 μl, 0.865 mmol) was delivered. The mixture was allowed to stir at 20° C. for 1.5 hrs. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1N NaOH and brine. The organic layer was passed through a phase separator and concentrated to give the crude product, 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-diazaheptapentacontyl)-3,6,9,12-tetraoxapentadecan-15-amide (63 mg, 0.056 mmol, 64.4% yield), as a clear colorless oil. Purity was judged >90% and the analytical characterization data of the final material was consistent with the assigned structure. All of this material was carried on as a synthetic intermediate. MS (ES) m/e 1018.9 [M+H]+, rt=0.63 mins.

### Step 2 (Compound 4)

### N1-((R)-1-(4-(4-chlorophenyl)piperidin-1-yl)-3-methyl-1-oxobutan-2-yl)-N3-(15,53-dioxo-57-((3aS,4S,6aR)-2-oxohexahydro-1H-thieno[3,4-d]imidazol-4-yl)-3,6,9,12,19,22,25,28,31,34, 37,40,43,46,49-pentadecaoxa-16,52-diazaheptapentacontyl)isophthalamide

Under a nitrogen atmosphere, a solution of (R)-3-((1-(4-(4-chlorophenyl)piperidin-1-yl)-3-methyl-1-oxobutan-2-yl)carbamoyl)benzoic acid (27 mg, 0.061 mmol), HATU (23.41 mg, 0.062 mmol), and HOBt (0.933 mg, 6.10 μmol) dissolved in N,N-Dimethylformamide (DMF) (610 μl) was prepared at room temperature in a small 4 mL vial. The mixture was treated with DIEA (31.9 μl, 0.183 mmol) and appeared to be some what homogeneous. Afterwards, to the mixture was added 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-3,6,9,12-tetraoxapentadecan-15-amide (62.1 mg, 0.061 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was purified by reverse phase HPLC [40-70% acetonitrile:water (0.1% NH4OH modifier), C18 50×30 mm GEMINI column, 47 mL/min]. All fractions containing the desired product were combined and concentrated to give the desired product, N1-((R)-1-(4-(4-chlorophenyl)piperidin-1-yl)-3-methyl-1-oxobutan-2-yl)-N3-(15,53-dioxo-57-((3aS,4S,6aR)-2-oxohexahydro-1H-thieno[3,4-d]imidazol-4-yl)-3,6,9,12,19,22,25,28,31,34,37,40,43,46,49-pentadecaoxa-16,52-diazaheptapentacontyl)isophthalamide (45 mg, 0.030 mmol, 48.6% yield), as a clear colorless oil after lyopholizing. Purity was judged >95% and the analytical characterization data of the final material was consistent with the assigned structure. All of this material was submitted for biological testing and storage. MS (ES) m/e 1443.2 [M+H]+, rt=1.07 mins. ¹H NMR (400 MHz, DMSO-d₆) □ ppm 8.54-8.66 (m, 2H) 8.34 (d, J=10.76 Hz, 1H) 8.00 (dd, J=18.22, 6.97 Hz, 2H) 7.78-7.90 (m, 2H) 7.55 (t, J=7.58 Hz, 1H) 7.18-7.39 (m, 3H) 6.41 (br. s., 1H) 4.80 (t, J=8.56 Hz, 1H) 4.59 (d, J=12.72 Hz, 1H) 4.23-4.40 (m, 2H) 4.12 (dd, J=7.82, 4.40 Hz, 1H) 3.35-3.61 (m, 63H) 3.05-3.22 (m, 6H) 2.82 (dd, J=12.47, 5.14 Hz, 2H) 2.54-2.74 (m, 2H) 2.30 (t, J=6.48 Hz, 2H) 2.21 (dt, J=13.88, 7.12 Hz, 1H) 2.02-2.09 (m, 3H) 1.72-1.94 (m, 2H) 1.22-1.66 (m, 8H) 0.89-1.00 (m, 6H)

### N1-((R)-1-(4-(4-chlorophenyl)piperidin-1-yl)-3-methyl-1-oxobutan-2-yl)-N3-(27,65-dioxo-69-((3aS,4S,6aR)-2-oxohexahydro-1H-thieno[3,4-d]imidazol-4-yl)-3,6,9,12,15,18,21,24,31,34, 37,40,43,46,49,52,55,58,61-nonadecaoxa-28,64-diazanonahexacontyl)isophthalamide

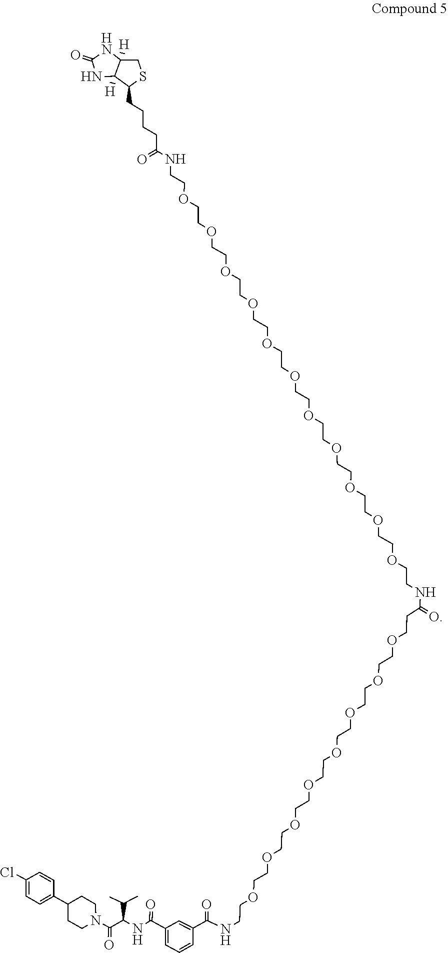 [SMILES: CC(C)[C@@H](NC(=O)c1cccc(C(=O)NCCOCCOCCOCCOCCOCCOCCOCCOCCC(=O)NCCOCCOCCOCCOCCOCCOCCOCCOCCOCCOCCOCCNC(=O)CCCC[C@@H]2SC[C@@H]3NC(=O)N[C@@H]32)c1)C(=O)N1CCC(c2ccc(Cl)cc2)CC1]

### Repeated Steps 1-4 Described for Compound 1

### Step 1 (Compound 5)

### 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-diazanonahexacontyl)-3,6,9,12,15,18,21,24-octaoxaheptacosan-27-amide

Under a nitrogen atmosphere, a solution of 2,2-dimethyl-4-oxo-3,8,11,14,17,20,23,26,29-nonaoxa-5-azadotriacontan-32-oic acid (42.2 mg, 0.078 mmol), HATU (29.6 mg, 0.078 mmol), and HOBt (11.92 mg, 0.078 mmol) was prepared in Dichloromethane (DCM) (778 μl) at ambient temperature. Afterwards, DIEA (45.3 μl, 0.259 mmol) was delivered. The mixture was homogeneous. After 10 mins the mixture was treated with N-(35-amino-3,6,9,12,15,18,21,24,27,30,33-undecaoxapentatriacontyl)-5-((3aS,4S,6aR)-2-oxohexahydro-1H-thieno[3,4-d]imidazol-4-yl)pentanamide (60 mg, 0.078 mmol) and was allowed to stir for 72 hours. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1:1 mixture of saturated sodium bicarbonate and water and brine. The organic layer was passed through a phase separator and concentrated to give the desired product, tert-butyl (27,65-dioxo-69-((3aS,4S,6aR)-2-oxohexahydro-1H-thieno[3,4-d]imidazol-4-yl)-3,6,9,12,15,18,21,24,31,34,37,40,43,46,49,52,55,58,61-nonadecaoxa-28,64-diazanonahexacontyl)carbamate (111 mg, 0.077 mmol, 99% yield), as a clear colorless oil. Purity was judged >90% and the analytical characterization data of the final material was consistent with the assigned structure. MS (ES) m/e 1295.1 [M+H]+, rt=0.87 mins.

Again, under a nitrogen atmosphere, Under a nitrogen atmosphere, a solution of (N43755-69-101) tert-butyl (27,65-dioxo-69-((3aS,4S,6aR)-2-oxohexahydro-1H-thieno[3,4-d]imidazol-4-yl)-3,6,9,12,15,18,21,24,31,34,37,40,43,46,49,52,55,58,61-nonadecaoxa-28,64-diazanonahexacontyl)carbamate (111 mg, 0.077 mmol) in 1,4 dioxane (1000 μl) was prepared at room temperature in a small microwave vial. The mixture was treated with 4M solution of HCl in dioxane (195 μl, 0.778 mmol) and appeared to be some what homogeneous. Finally, the reaction was stirred at room temperature for 18 hours. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was diluted with DCM and treated with an aqueous work up using 1N NaOH and brine. The organic layer was passed through a phase separator and concentrated to give the crude product, 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-diazanonahexacontyl)-3,6,9,12,15,18,21,24-octaoxaheptacosan-27-amide (80 mg, 0.06 mmol, 77% yield), as a clear colorless oil. Purity was judged >90% and the analytical characterization data of the final material was consistent with the assigned structure. All of this material was carried on as a synthetic intermediate. MS (ES) m/e 1195 [M+H]+, rt=0.67 mins.

### Step 2 (Compound 5)

### N1-((R)-1-(4-(4-chlorophenyl)piperidin-1-yl)-3-methyl-1-oxobutan-2-yl)-N3-(27,65-dioxo-69-((3aS,4S,6aR)-2-oxohexahydro-1H-thieno[3,4-d]imidazol-4-yl)-3,6,9,12,15,18,21,24,31,34, 37,40,43,46,49,52,55,58,61-nonadecaoxa-28,64-diazanonahexacontyl)isophthalamide

Under a nitrogen atmosphere, a solution of (R)-3-((1-(4-(4-chlorophenyl)piperidin-1-yl)-3-methyl-1-oxobutan-2-yl)carbamoyl)benzoic acid (26 mg, 0.059 mmol), HATU (22.54 mg, 0.059 mmol), and HOBt (0.899 mg, 5.87 μmol) dissolved in N,N-Dimethylformamide (DMF) (587 μl) was prepared at room temperature in a small 4 mL vial. The mixture was treated with DIEA (30.8 μl, 0.176 mmol) and appeared to be some what homogeneous. Afterwards, to the mixture was added 1-amino-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-3,6,9,12,15,18,21,24-octaoxaheptacosan-27-amide (70.1 mg, 0.059 mmol). Finally, the reaction was stirred at room temperature for 18 hr. The progress was checked by Open Access LCMS [Shimadzu 10A PE (LC) coupled with Sciex Single Quadrupole 150EX (MS) and Sedere Sedex 75C (ELS)] ((Thermo Hypersil Gold C18, 20×2.1 mm, 1.9 u particle diam.), 1.6 mL/min, gradient from 5-92% CH3CN (0.02% TFA)/H2O (0.02% TFA)). The spectra confirmed that the reaction was progressed fairly cleanly and was about 100% complete. The reaction mixture was purified by reverse phase HPLC [40-70% acetonitrile:water (0.1% NH4OH modifier), C18 50×30 mm GEMINI column, 47 mL/min]. All fractions containing the desired product were combined and concentrated to give the desired product, N1-((R)-1-(4-(4-chlorophenyl)piperidin-1-yl)-3-methyl-1-oxobutan-2-yl)-N3-(27,65-dioxo-69-((3aS,4S,6aR)-2-oxohexahydro-1H-thieno[3,4-d]imidazol-4-yl)-3,6,9,12,15,18,21,24,31,34,37,40,43,46,49,52,55,58,61-nonadecaoxa-28,64-diazanonahexacontyl)isophthalamide (41 mg, 0.024 mmol, 41.0% yield), as a yellow oil after lyopholizing. Purity was judged >95% and the analytical characterization data of the final material was consistent with the assigned structure. All of this material was submitted for biological testing and storage. MS (ES) m/e 1619[M+H]+, rt=1.09 mins. ¹H NMR (400 MHz, DMSO-d₆) □ ppm 8.54-8.66 (m, 2H) 8.34 (d, J=11.00 Hz, 1H) 8.00 (dd, J=17.97, 7.21 Hz, 2H) 7.78-7.90 (m, 2H) 7.55 (t, J=7.70 Hz, 1H) 7.18-7.39 (m, 4H) 6.41 (br. s., 1H) 4.80 (t, J=8.56 Hz, 1H) 4.58 (d, J=12.47 Hz, 1H) 4.23-4.40 (m, 2H) 4.12 (dd, J=7.70, 4.52 Hz, 1H) 3.36-3.60 (m, 76H) 3.05-3.24 (m, 6H) 2.82 (dd, J=12.47, 5.14 Hz, 2H) 2.54-2.75 (m, 3H) 2.31 (t, J=6.48 Hz, 2H) 2.21 (dt, J=14.24, 7.18 Hz, 1H) 2.03-2.10 (m, 2H) 1.21-1.95 (m, 12H) 0.88-1.00 (m, 6H)

### CCR2

### N-(29-((2,4-dinitrophenyl)amino)-3,6,9,12,15,18,21,24,27-nonaoxanonacosyl)-4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide, Trifluoroacetic acid salt

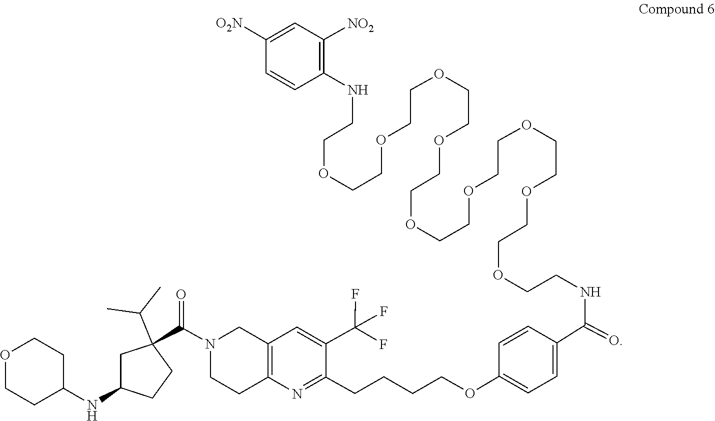 [SMILES: CC(C)[C@]1(C(=O)N2CCc3nc(CCCCOc4ccc(C(=O)NCCOCCOCCOCCOCCOCCOCCOCCOCCOCCNc5ccc([N+](=O)[O-])cc5[N+](=O)[O-])cc4)c(C(F)(F)F)cc3C2)CC[C@@H](NC2CCOCC2)C1]

### Step 1

### (2-(4-hydroxybutyl)-3-(trifluoromethyl)-7,8-dihydro-1,6-naphthyridin-6 (5H)-yl)((1S,3R)-1-isopropyl-3-((tetrahydro-2H-pyran-4-yl)amino)cyclopentyl)methanone

Method reference Jin, J.; MacMillan, D. W. C. Nature 2015, 525, 87-90

In a 20 mL vial tosicacid (529 mg, 2.78 mmol), ((1S,3R)-1-isopropyl-3-((tetrahydro-2H-pyran-4-yl)amino)cyclopentyl)(3-(trifluoromethyl)-7,8-dihydro-1,6-naphthyridin-6 (5H)-yl)methanone, 2Hydrochloride (570 mg, 1.112 mmol) (WO2003092586A2), and Ir(dtbpy)(bpy)2PF6 (17 mg, 0.019 mmol) were dissolved in DMSO (4.4 mL) and THF (4.4 mL). To this was added ethyl 2-mercaptopropanoate (30 μl, 0.23 mmol). The solution was split into two 2 dram vials and the resulting solution was irradiated with a blue LED lamp (Kessil H150 blue) at a distance of ˜2 cm for 1 week.

The reaction was concentrated under a stream of nitrogen at 50° C. then diluted with water and washed with EtOAc (three times). The aqueous layer was basified with 1 N NaOH and extracted with DCM (4 times). The combined organic layers were washed with brine (twice) then the organic layer was dried over anhydrous sodium sulfate, filtered, and concentrated under a stream of nitrogen at 50° C. The resultant residue was dissolved in DMSO and purified on a x-bridge prep C18 5 □m OBD 30×150 mm column with a gradient from 10-90% ACN/(0.1% NH₄OH/H₂O) over 10 min. The desired fractions were collected and concentrated under a stream of nitrogen at 50° C. resulting in (2-(4-hydroxybutyl)-3-(trifluoromethyl)-7,8-dihydro-1,6-naphthyridin-6 (5H)-yl)((1S,3R)-1-isopropyl-3-((tetrahydro-2H-pyran-4-yl)amino)cyclopentyl)methanone (116 mg, 0.177 mmol, 15.9% yield) as a clear gum.

LC/MS (ESI): m/z 512.2 (M+H)⁺, 0.72 min (ret. time)

¹H NMR (400 MHz, METHANOL-d₄) indicated a mixture of the starting material (12%) with the desired product (78%) and the alternative regioisomer (10%). The material was carried on as a mixture.

### Step 2

### Methyl 4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate

At 10° C., (2-(4-hydroxybutyl)-3-(trifluoromethyl)-7,8-dihydro-1,6-naphthyridin-6 (5H)-yl)((1S,3R)-1-isopropyl-3-((tetrahydro-2H-pyran-4-yl)amino)cyclopentyl)methanone (17 mg, 0.033 mmol), triphenylphosphine (11 mg, 0.04 mmol), and methyl 4-hydroxybenzoate (6 mg, 0.04 mmol) were dissolved in Tetrahydrofuran (THF) (0.2 mL). DEAD (40 wt. % in toluene) (0.02 mL, 0.04 mmol) was added and the reaction was heated to 40° C. for 3.5 h. The reaction was added to isolute and concentrated under a stream of nitrogen at 50° C. The crude product was purified on a silica cartridge (4 g) with a Combiflash Rf 200i, eluting at 18 mL/min with a non-linear 0-100% (2% NH₄OH in 3:1 EtOH/EtOAc)/hexanes gradient. The desired fractions were concentrated under reduced pressure and dried under high vacuum, giving methyl 4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate (11 mg, 0.012 mmol, 37.4% yield) as a tan film. Two peaks in LCMS at 1.07 and 1.11 min correspond to regiosomers from the previous reaction carried on as a mixture.

LC/MS (ESI): m/z 646.3 (M+H)⁺, 1.07 min and 1.11 min (ret. time) minor isomer accounts for roughly 15% of the product UV AUC.

### Step 3

### N-(29-((2,4-dinitrophenyl)amino)-3,6,9,12,15,18,21,24,27-nonaoxanonacosyl)-4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide

Methyl 4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate (11 mg, 0.017 mmol) and lithium hydroxide hydrate (1.4 mg, 0.034 mmol) were dissolved in methanol (0.06 mL), water (0.06 mL), and THF (0.06 mL). The reaction was shaken at 50° C. for 1.5 h then concentrated under a stream of nitrogen at 50° C. Water (approximately 1 mL) was added to the resultant residue and the mixture was lyophilized resulting in a orange solid. TSTU (6.2 mg, 0.020 mmol) was added and the reaction was dissolved in DMF (0.2 mL) and TEA (0.01 mL, 0.072 mmol). After 2.5 h, N1-(2,4-dinitrophenyl)-3,6,9,12,15,18,21,24,27-nonaoxanonacosane-1,29-diamine (13 mg, 0.020 mmol) dissolved in N,N-Dimethylformamide (DMF) (0.2 mL) was added to the reaction, and stirred for 45 min. The reaction was diluted with MeOH and purified on a Gilson HPLC (Sunfire 5 μm C18 OBD 19×100 mm preparatory column), eluting at 20 mL/min with a linear gradient running from 10-90% CH₃CN/H₂O (0.1% TFA) over 12 min. The desired fractions were concentrated under a stream of nitrogen at 50° C., giving lot A1 of N-(29-((2,4-dinitrophenyl)amino)-3,6,9,12,15,18,21,24,27-nonaoxanonacosyl)-4-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide, Trifluoroacetic acid salt (4.5 mg, 3.33 μmol, 19.56% yield) as a yellow film.

LC/MS (ESI): m/z 619.1 (M+2H)⁺⁺, 1.11 min (ret. time)

HPLC: 13.323 min (ret. time); Luna C18 (2) 4.6×150 mm, 3 □m. 2-95% (0.1% TFA in ACN)/water over 18 min, held 95% for 2 min

### N-(29-((2,4-dinitrophenyl)amino)-3,6,9,12,15,18,21,24,27-nonaoxanonacosyl)-3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide, 2×Trifluoroacetic acid salt

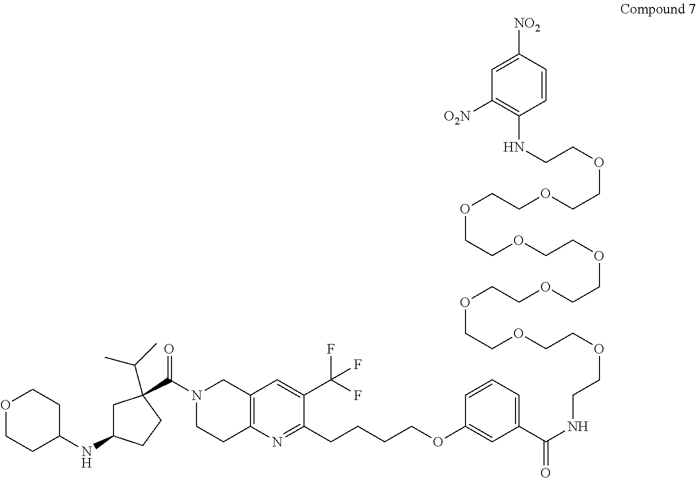 [SMILES: CC(C)[C@]1(C(=O)N2CCc3nc(CCCCOc4cccc(C(=O)NCCOCCOCCOCCOCCOCCOCCOCCOCCOCCNc5ccc([N+](=O)[O-])cc5[N+](=O)[O-])c4)c(C(F)(F)F)cc3C2)CC[C@@H](NC2CCOCC2)C1]

### Step 1

### Methyl 3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate

To (2-(4-hydroxybutyl)-3-(trifluoromethyl)-7,8-dihydro-1,6-naphthyridin-6 (5H)-yl)((1S,3R)-1-isopropyl-3-((tetrahydro-2H-pyran-4-yl)amino)cyclopentyl)methanone (100 mg, 0.195 mmol) was added methyl 3-hydroxybenzoate (36 mg, 0.24 mmol) and triphenylphosphine (62 mg, 0.24 mmol). The solids were dissolved in THF (1 mL) and DEAD (40 wt. % in toluene) (0.107 mL, 0.235 mmol) was added slowly, dropwise. The reaction was stirred at room temperature for 1.25 h then concentrated onto isolute under reduced pressure. The crude product was purified on a silica cartridge (12 g, gold) with a Combiflash Rf 200i, eluting at 35 mL/min with a non-linear 0 to 60% (2% NH₄OH in 3:1 EtOH/EtOAc)/hexanes gradient. The desired fractions were concentrated under reduced pressure resulting in a tan film (118 mg). LCMS indicated the minor regioisomer from the previous reaction was still present. The film was dissolved in DMSO (1.5 mL), and purified on a Gilson HPLC (Sunfire 5 μm C18 OBD 30×100 mm preparatory column), eluting at 30 mL/min with a linear gradient running from 20-90% CH₃CN/H₂O (0.1% TFA) over 16 min. The fractions containing the major isomer were combined and diluted with saturated sodium bicarbonate, then extracted with DCM (3 times). The combined DCM layers were washed with brine, dried over anhydrous sodium sulfate, filtered, and concentrated under reduced pressure, resulting in methyl 3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate (45 mg, 0.070 mmol, 35.7% yield) as a clear film.

LC/MS (ESI): m/z 646.2 (M+H)⁺, 1.09 min (ret. time)

### Step 2

### 3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoic acid

Methyl 3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoate (45 mg, 0.070 mmol) and lithium hydroxide hydrate (3 mg, 0.07 mmol) were dissolved in methanol (0.23 mL), water (0.23 mL), and THF (0.23 mL). The reaction was shaken at 50° C. for 1 h then concentrated under a stream of nitrogen at 50° C. overnight. A second aliquot of lithium hydroxide hydrate (0.8 mg, 0.02 mmol) and methanol (0.23 mL), water (0.23 mL), and THF (0.23 mL) were added and the reaction concentrated under a stream of nitrogen at 50° C. A third aliquot of lithium hydroxide hydrate (3 mg, 0.07 mmol) was added, followed by methanol (0.23 mL), water (0.23 mL), and THF (0.23 mL). The reaction was shaken at 50° C. for 45 min then concentrated under a stream of nitrogen at 50° C. resulting in 3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoic acid (30 mg, 0.047 mmol, 68.1% yield) as a clear film.

LC/MS (ESI): m/z 632.3 (M+H)⁺, 0.97 min (ret. time)

### Step 3

### N-(29-((2,4-dinitrophenyl)amino)-3,6,9,12,15,18,21,24,27-nonaoxanonacosyl)-3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide, 2×Trifluoroacetic acid salt

3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzoic acid, Lithium salt (8.8 mg, 0.014 mmol) and TSTU (4.5 mg, 0.015 mmol) were dissolved in DMSO (0.3 mL) and TEA (0.01 mL, 0.07 mmol). The reaction was sonicated for 1 min then N1-(2,4-dinitrophenyl)-3,6,9,12,15,18,21,24,27-nonaoxanonacosane-1,29-diamine (11 mg, 0.018 mmol) dissolved in DMSO (0.6 mL) and TEA (0.02 mL, 0.14 mmol) was added, and the reaction was sonicated for 1 min. The reaction was transferred with AcOH to a test tube then purified on a Gilson HPLC (Sunfire 5 μm C18 OBD 30×100 mm preparatory column), eluting at 30 mL/min with a linear gradient running from 10-90% CH₃CN/H₂O (0.1% TFA) over 12 min. The desired fractions were concentrated under a stream of nitrogen at 50° C., giving N-(29-((2,4-dinitrophenyl)amino)-3,6,9,12,15,18,21,24,27-nonaoxanonacosyl)-3-(4-(6-((1S,3R)-1-isopropyl-3-((tetrahydro-2H-pyran-4-yl)amino)cyclopentanecarbonyl)-3-(trifluoromethyl)-5,6,7,8-tetrahydro-1,6-naphthyridin-2-yl)butoxy)benzamide, 2×Trifluoroacetic acid salt (9.8 mg, 6.69 μmol, 48.6% yield) as a yellow film.

LC/MS (ESI): m/z 618.9 (M+2H)⁺⁺, 1.12 min (ret. time)

HPLC: 13.481 min (ret. time) on a Luna C18 (2) 4.6×150 mm, 3 μm. 2% B to 95% B in 18 min, hold at 95% B for 2 min, acidic conditions. 0.1% TFA in ACN (% B) and H₂O (% A)

### N1-(2-(2-(2-(2-((2-(4-((S)-2-((3S,3'S)-3′,6-Dimethylspiro[indoline-3,4′-piperidin]-1′-yl)-1-hydroxyethyl)piperidine-1-carbonyl)-1H-indol-5-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide

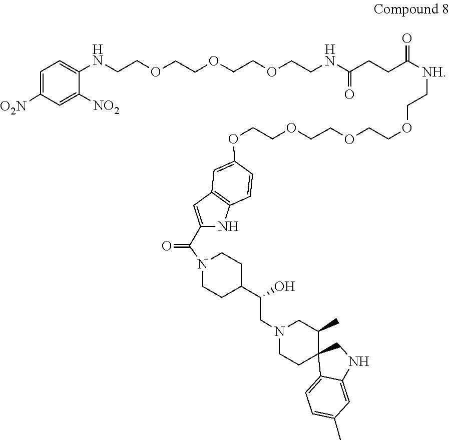 [SMILES: Cc1ccc2c(c1)NC[C@]21CCN(C[C@@H](O)C2CCN(C(=O)c3cc4cc(OCCOCCOCCOCCNC(=O)CCC(=O)NCCOCCOCCOCCNc5ccc([N+](=O)[O-])cc5[N+](=O)[O-])ccc4[nH]3)CC2)C[C@H]1C]

### Step 1

### 1-((2,4-dinitrophenyl)amino)-13-oxo-3,6,9-trioxa-12-azahexadecan-16-oic acid

To a solution of tert-butyl (2-(2-(2-(2-aminoethoxy)ethoxy)ethoxy)ethyl)carbamate (1.0 g, 3.42 mmol) in dichloromethane (DCM) (10.0 mL) was added 1-fluoro-2,4-dinitrobenzene (0.429 mL, 3.42 mmol), followed by DIPEA (0.896 mL, 5.13 mmol) and the mixture was stirred at rt for 3 h. After this period, TFA (2.64 mL, 34.2 mmol) was added and the mixture was stirred for 16 h before it was concentrated in vacuo. The evaporation residue was partitioned between aq. NH₄OH and DCM. The organic layer was separated, concentrated in vacuo, and then azeotropically dried by toluene.

The dry evaporated residue was dissolved in dichloromethane (DCM) (10.00 mL) and stirred with succinic anhydride (0.342 g, 3.42 mmol) for 24 h. The reaction mixture was diluted with DCM and washed with aqueous sodium dihydrogenphosphate. The organic layer was separated, dried over Na₂SO₄, and concentrated in vacuo to afford 1-((2,4-dinitrophenyl)amino)-13-oxo-3,6,9-trioxa-12-azahexadecan-16-oic acid (982 mg, 2.142 mmol, 62.6% yield) as a yellow oil. ¹H NMR (400 MHz, CHLOROFORM-d) δ ppm 10.11 (br. s, 1H), 9.15 (d, J=2.69 Hz, 1H), 8.82 (br. s., 1H), 8.30 (dd, J=9.41, 2.57 Hz, 1H), 6.98 (d, J=9.54 Hz, 1H), 6.56 (br. s., 1H), 3.86 (t, J=5.14 Hz, 2H), 3.54-3.81 (m, 12H), 3.48 (q, J=5.22 Hz, 2H), 2.67-2.73 (m, 2H), 2.53-2.59 (m, 2H). LCMS (2 min TFA): Rt=0.73 min, [M+H]⁺=459.0.

### Step 2

### N1-(2-(2-(2-(2-((2,4-Dinitrophenyl)amino)ethoxy)ethoxy)ethoxy)ethyl)-N4-(2-(2-(2-(2-hydroxyethoxy)ethoxy)ethoxy)ethyl)succinamide

The a solution of 1-((2,4-dinitrophenyl)amino)-13-oxo-3,6,9-trioxa-12-azahexadecan-16-oic acid (980 mg, 2.138 mmol) in N,N-dimethylformamide (DMF) (20.00 mL), was added HATU (1.301 g, 3.42 mmol) followed by DIPEA (2.383 mL, 13.68 mmol), and the resulting mixture was stirred at rt for 5 min before 2-(2-(2-(2-aminoethoxy)ethoxy)ethoxy)ethanol (454 mg, 2.352 mmol) was added and the mixture was stirred for 16 h. After this period, water (0.5 mL) was added and the mixture was concentrated in vacuo (30° C., 0-1 mbar vacuum). The evaporation residue was taken up in DCM and washed consecutively with aq. NaH₂PO₄, water, and sat. aq. NaHCO₃. The organic layer was separated, dried over Na₂SO₄, and concentrated in vacuo. The evaporation residue was subjected to normal phase purification on a Biotage Ultra SNAP 100 g SiO2 column (2-15% MeOH/DCM) to afford N1-(2-(2-(2-(2-((2,4-dinitrophenyl)amino)ethoxy)ethoxy)ethoxy)ethyl)-N4-(2-(2-(2-(2-hydroxyethoxy)ethoxy)ethoxy)ethyl)succinamide (456 mg, 0.720 mmol, 33.7% yield) as a viscous bright yellow oil. ¹H NMR (400 MHz, CHLOROFORM-d) □ ppm 9.09 (d, J=2.69 Hz, 1H), 8.79 (br. s., 1H), 8.24 (dd, J=9.54, 2.20 Hz, 1H), 7.24 (br. t, J=4.80, 4.80 Hz, 1H), 6.98 (d, J=9.54 Hz, 1H), 6.59 (br. t, J=5.00, 5.00 Hz, 1H), 3.83 (t, J=5.26 Hz, 2H), 3.48-3.75 (m, 26H), 3.35-3.44 (m, 4H), 2.50 (t, J=2.57 Hz, 4H). LCMS (2 min TFA): Rt=0.75 min, [M+H]⁺=634.1.

### Step 3

### 5-((28-((2,4-Dinitrophenyl)amino)-13,16-dioxo-3,6,9,20,23,26-hexaoxa-12,17-diazaoctacosyl)oxy)-1H-indole-2-carboxylic acid

To a solution of N1-(2-(2-(2-(2-((2,4-dinitrophenyl)amino)ethoxy)ethoxy)ethoxy)ethyl)-N4-(2-(2-(2-(2-hydroxyethoxy)ethoxy)ethoxy)ethyl)succinamide (248 mg, 0.391 mmol) in pyridine (3.0 mL) was added 4-methylbenzene-1-sulfonyl chloride (74.6 mg, 0.391 mmol) and the resulting solution was stirred at rt for 4 h. After this period, pyridine was removed in vacuo.

The evaporation residue was dissolved in N,N-dimethylformamide (DMF) (5.00 mL), ethyl 5-hydroxy-1H-indole-2-carboxylate (120 mg, 0.587 mmol), and cesium carbonate (638 mg, 1.957 mmol) were added, and the resulting mixture was stirred at rt for 3 h. After this period, DMF was removed in vacuo, the evaporation residue taken up in DCM (with a few drops of MeOH), filtered, concentrated in vacuo, and subjected to normal phase purification (0-20% MeOH/DCM) to afford 60 mg of the intermediate ethyl ester.

The above ethyl ester was dissolved in methanol (3.00 mL), 1.0 M sodium hydroxide (1.0 mL, 1.000 mmol) was added, and the mixture was stirred at rt for 19 h. After this period, 1.0 M HCl (1.0 mL) was added, the mixture was concentrated in vacuo, the evaporation residue taken up in 1:1 DCM/iPrOH, filtered, and concentrated in vacuo again to afford crude 5-((28-((2,4-dinitrophenyl)amino)-13,16-dioxo-3,6,9,20,23,26-hexaoxa-12,17-diazaoctacosyl)oxy)-1H-indole-2-carboxylic acid (60 mg, 0.076 mmol, 19.34% yield). LCMS (2 min TFA): Rt=0.90 min, [M+H]⁺=793.0.

### Step 4

### N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-5-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide

To a solution of 5-((28-((2,4-dinitrophenyl)amino)-13,16-dioxo-3,6,9,20,23,26-hexaoxa-12,17-diazaoctacosyl)oxy)-1H-indole-2-carboxylic acid (60 mg, 0.076 mmol (WO 2008/157741 A1, PCT/US2008/067589) in N,N-dimethylformamide (DMF) (1.00 mL) was added HATU (28.8 mg, 0.076 mmol), followed by DIPEA (0.066 mL, 0.378 mmol), and the resulting mixture was stirred at rt for 5 min. After this period, a solution of (S)-2-((3S,3'S)-3′,6-dimethylspiro[indoline-3,4′-piperidin]-1′-yl)-1-(piperidin-4-yl)ethanol (26.0 mg, 0.076 mmol) in N,N-dimethylformamide (DMF) (1.00 mL) was added and the mixture was stirred at rt for 15 minutes before it was quenched by water (0.2 mL) and stirred at rt for 15 min. The mixture was concentrated in vacuo and subjected to normal phase purification on a Biotage Ultra SNAP 50 g silicagel column (0-20% MeOH/DCM) to yield N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-5-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide (27 mg, 0.024 mmol, 31.9% yield) as a yellow oil. ¹H NMR (400 MHz, METHANOL-d₄) □ ppm 9.04 (d, J=2.69 Hz, 1H), 8.23 (dd, J=9.54, 2.69 Hz, 1H), 7.53 (s, 1H), 7.31 (d, J=8.80 Hz, 1H), 7.00-7.10 (m, 2H), 6.83-6.96 (m, 2H), 6.66 (s, 1H), 6.55 (d, J=7.58 Hz, 1H), 6.46 (s, 1H), 4.60-4.71 (m, 2H), 4.10-4.17 (m, 2H), 3.46-3.89 (m, 34H), 3.34 (m, J=4.40 Hz, 2H), 2.96-3.06 (m, 2H), 2.55-2.68 (m, 2H), 2.44 (s, 4H), 2.22 (s, 3H), 1.90-2.08 (m, 4H), 1.66-1.83 (m, 3H), 1.37-1.51 (m, 2H), 0.68 (d, J=6.11 Hz, 2H). LCMS (2 min TFA): Rt=0.89 min, [M/2+H]⁺=560.0.

### (E)-1-(4-((S)-2-((3S,3'S)-3′,6-Dimethylspiro[indoline-3,4′-piperidin]-1′-yl)-1-hydroxyethyl)piperidin-1-yl)-3-(3-((29-((2,4-dinitrophenyl)amino)-3,6,9,12,15,18,21,24,27-nonaoxanonacosyl)oxy)-5-fluorophenyl)prop-2-en-1-one

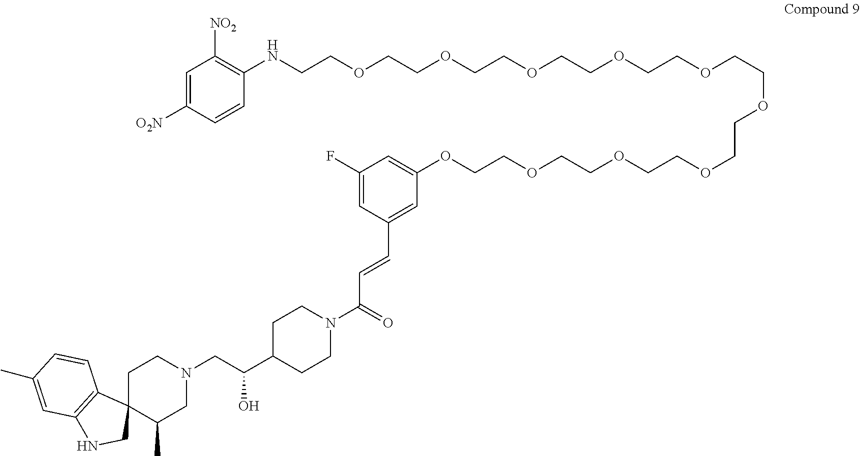 [SMILES: Cc1ccc2c(c1)NC[C@]21CCN(C[C@@H](O)C2CCN(C(=O)/C=C/c3cc(F)cc(OCCOCCOCCOCCOCCOCCOCCOCCOCCOCCNc4ccc([N+](=O)[O-])cc4[N+](=O)[O-])c3)CC2)C[C@H]1C]

### Step 1

### 2,2-Dimethyl-4-oxo-3,8,11,14,17,20,23,26,29,32-decaoxa-5-azatetratriacontan-34-yl 4-methylbenzenesulfonate

To a solution of 29-amino-3,6,9,12,15,18,21,24,27-nonaoxanonacosan-1-ol (1.0 g, 2.186 mmol) in tetrahydrofuran (THF) (4.0 mL) was added DMAP (0.013 g, 0.109 mmol), followed by 2.0 M Boc-anhydride in THF (1.147 mL, 2.295 mmol) and triethylamine (0.609 mL, 4.37 mmol) and the resulting mixture was stirred in an open flask at rt for 30 min. After this period, the mixture was heated gently with a heat gun until THF started boiling. The heating was stopped and the mixture was stirred for an additional 30 min while gradually cooling to rt. The mixture was concentrated in vacuo and azetroped with toluene (2×).

To a solution of the above Boc-protected intermediate in pyridine (5.00 mL) was added Ts-Cl (0.437 g, 2.295 mmol) at rt in one portion and the mixture was stirred at rt for 24 h. After this period, pyridine was removed in vacuo, the evaporation residue partitioned between ethyl acetate and a 1:1 mixture of brine and water, the organic layer dried over sodium sulfate and concentrated in vacuo to afford crude 2,2-dimethyl-4-oxo-3,8,11,14,17,20,23,26,29,32-decaoxa-5-azatetratriacontan-34-yl 4-methylbenzenesulfonate (1.03 g, 1.447 mmol, 66.2% yield) as a colorless oil. LCMS (2 min TFA): Rt=1.12 min, [M+H]⁺=734.2.

### Step 2

### (E)-Ethyl 3-(3-fluoro-5-hydroxyphenyl)acrylate

To a suspension of 3-fluoro-5-hydroxybenzaldehyde (980 mg, 6.99 mmol) in toluene (10.00 mL) was added potassium carbonate (1933 mg, 13.99 mmol) and triethyl phosphonoacetate (1.540 mL, 7.69 mmol) and the mixture was stirred at rt for 3 days. After this period, water (100 mL) was added, and the mixture was extracted with ethyl acetate (3×100 mL). The combined organic extractes were dried over magnesium sulfate, filtered, concentrated in vacuo, and subjected normal phase purification on a Biotage Ultra SNAP 100 g silica column (0-30% EtOAc/hexanes) to yield (E)-ethyl 3-(3-fluoro-5-hydroxyphenyl)acrylate (1.105 g, 5.26 mmol, 75% yield) as a white powder. ¹H NMR (400 MHz, DMSO-d₆) □ ppm 10.11 (br. s., 1H), 7.53 (d, J=15.89 Hz, 1H), 7.06 (dt, J=9.66, 1.77 Hz, 1H), 6.88 (d, J=1.47 Hz, 1H), 6.54-6.66 (m, 2H), 4.19 (q, J=7.09 Hz, 2H), 1.25 (t, J=7.09 Hz, 3H). LCMS (2 min TFA): Rt=0.88 min, [M+H]⁺=211.2.

### Step 3

### (E)-ethyl 3-(3-((2,2-dimethyl-4-oxo-3,8,11,14,17,20,23,26,29,32-decaoxa-5-azatetratriacontan-34-yl)oxy)-5-fluorophenyl)acrylate

To a solution of (E)-ethyl 3-(3-fluoro-5-hydroxyphenyl)acrylate (0.608 g, 2.89 mmol) in N,N-dimethylformamide (DMF) (10.0 mL) was added 2,2-dimethyl-4-oxo-3,8,11,14,17,20,23,26,29,32-decaoxa-5-azatetratriacontan-34-yl 4-methylbenzenesulfonate (1.03 g, 1.447 mmol) followed by cesium carbonate (2.357 g, 7.23 mmol) and the mixture was stirred at rt for 24 h. After this period, the mixture was diluted with ethyl acetate (100 mL), the solids were filtered off, and the filtrate concentrated in vacuo. The evaporation residue was subjected to normal phase purification (0-20% MeOH/DCM) to afford (E)-ethyl 3-(3-((2,2-dimethyl-4-oxo-3,8,11,14,17,20,23,26,29,32-decaoxa-5-azatetratriacontan-34-yl)oxy)-5-fluorophenyl)acrylate (0.914 g, 1.219 mmol, 84% yield) as a colorless oil. LCMS (2 min TFA): Rt=1.26 min, [M+Na]⁺=772.2.

### Step 4

### (E)-Ethyl 3-(3-((29-amino-3,6,9,12,15,18,21,24,27-nonaoxanonacosyl)oxy)-5-fluorophenyl)acrylate, Trifluoroacetic acid salt

To a solution of (E)-ethyl 3-(3-((2,2-dimethyl-4-oxo-3,8,11,14,17,20,23,26,29,32-decaoxa-5-azatetratriacontan-34-yl)oxy)-5-fluorophenyl)acrylate (914 mg, 1.219 mmol) in dichloromethane (DCM) (10.0 mL) was added TFA (3.0 mL, 38.9 mmol) and the resulting mixture was stirred at rt for 20 min. The mixture was then concentrated in vacuo (0 mbar, 37 C) and the residual TFA was driven off by azetropic distillaton with PhMe to afford crude (E)-ethyl 3-(3-((29-amino-3,6,9,12,15,18,21,24,27-nonaoxanonacosyl)oxy)-5-fluorophenyl)acrylate, trifluoroacetic acid salt (1.00 g, 1.309 mmol, 107% yield) as a colorless oil. LCMS (2 min TFA): Rt=0.94 min, [M+H]⁺=650.3.

### Step 5

### (E)-Ethyl 3-(3-((29-((2,4-dinitrophenyl)amino)-3,6,9,12,15,18,21,24,27-nonaoxanonacosyl)oxy)-5-fluorophenyl)acrylate

To a solution of (E)-ethyl 3-(3-((29-amino-3,6,9,12,15,18,21,24,27-nonaoxanonacosyl)oxy)-5-fluorophenyl)acrylate, trifluoroacetic acid salt (500 mg, 0.655 mmol) in dichloromethane (DCM) (10.0 mL) was added 1-fluoro-2,4-dinitrobenzene (0.083 mL, 0.655 mmol) followed by triethylamine (0.456 mL, 3.27 mmol) and the resulting mixture was stirred at rt for 24 h. After this period, the mixture was washed with a 1:1 mixture of brine and and water, the organic layer was separated, dried over sodium sulfate, filtered, and concentrated in vacuo. The crude evaporation residue was purified on a Biotage Ultra SNAP 100 g silicagel column (2-7% MeOH/DCM) to afford (E)-ethyl 3-(3-((29-((2,4-dinitrophenyl)amino)-3,6,9,12,15,18,21,24,27-nonaoxanonacosyl)oxy)-5-fluorophenyl)acrylate (484 mg, 0.593 mmol, 91% yield) as a yellow-orange oil. LCMS (2 min TFA): Rt=1.32 min, [M+H]⁺=816.1.

### Step 6

### (E)-3-(3-((29-((2,4-Dinitrophenyl)amino)-3,6,9,12,15,18,21,24,27-nonaoxanonacosyl)oxy)-5-fluorophenyl)acrylic acid

To a solution of (E)-ethyl 3-(3-((29-((2,4-dinitrophenyl)amino)-3,6,9,12,15,18,21,24,27-nonaoxanonacosyl)oxy)-5-fluorophenyl)acrylate (484 mg, 0.593 mmol) in methanol (10.0 mL) was added 1.0 M NaOH (3.0 mL, 3.00 mmol) and the resulting mixture was stirred at rt for 24 h. After this period, 1.0 M HCl (3.0 mL, 3.00 mmol) was added, followed by toluene (20 mL) and the mixture was concentrated in vacuo (0 mbar, 37° C.). The evaporation residue was taken up in a 9:1 mixture of DCM/IPA, the insoluble NaCl was filtered off, the filtrate diluted with PhMe (50 mL) and DCM (15 mL), and concentrated in vacuo (0 mbar, 37° C., 1 h) to afford crude (E)-3-(3-((29-((2,4-dinitrophenyl)amino)-3,6,9,12,15,18,21,24,27-nonaoxanonacosyl)oxy)-5-fluorophenyl)acrylic acid (504 mg, 0.640 mmol, 108% yield). LCMS (2 min TFA): Rt=1.12 min, [M+H]⁺=788.1.

### Step 7

### E)-1-(4-((S)-2-((3S,3'S)-3′,6-Dimethylspiro[indoline-3,4′-piperidin]-1′-yl)-1-hydroxyethyl)piperidin-1-yl)-3-(3-((29-((2,4-dinitrophenyl)amino)-3,6,9,12,15,18,21,24,27-nonaoxanonacosyl)oxy)-5-fluorophenyl)prop-2-en-1-one

To a solution of (E)-3-(3-((29-((2,4-dinitrophenyl)amino)-3,6,9,12,15,18,21,24,27-nonaoxanonacosyl)oxy)-5-fluorophenyl)acrylic acid (172 mg, 0.218 mmol) in N,N-dimethylformamide (DMF) (2.00 mL) was added (S)-2-((3S,3′S)-3′,6-dimethylspiro[indoline-3,4′-piperidin]-1′-yl)-1-(piperidin-4-yl)ethanol (75 mg, 0.218 mmol), followed by HATU (83 mg, 0.218 mmol) and DIPEA (0.191 mL, 1.092 mmol) and the resulting mixture was stirred at rt for 1 h. After this period, the mixture was quenched by water (0.2 mL) and stirred at rt for 15 min. The mixture was then concentrated in vacuo, azetroped with DCM/MeOH/toluene (3×), and subjected to normal phase purification on a Biotage Ultra SNAP 100 g silicagel column (2-20% MeOH/DCM). The purified product was contaminated by a small amount of hydroxybenzotriazole (HATU artefact) which was removed by extraction of a CH₂C₂ solution of the product with 5% aqueous ammonia. The organic layer was separated and concentrated in vacuo to afford (E)-1-(4-((S)-2-((3S,3'S)-3′,6-dimethylspiro[indoline-3,4′-piperidin]-1′-yl)-1-hydroxyethyl)piperidin-1-yl)-3-(3-((29-((2,4-dinitrophenyl)amino)-3,6,9,12,15,18,21,24,27-nonaoxanonacosyl)oxy)-5-fluorophenyl)prop-2-en-1-one (104 mg, 0.093 mmol, 42.8% yield) as a yellow oil. ¹H NMR (400 MHz, METHANOL-d₄) □ ppm 9.02 (d, J=2.76 Hz, 1H), 8.26 (dd, J=9.54, 2.76 Hz, 1H), 7.43 (d, J=15.31 Hz, 1H), 7.07-7.22 (m, 2H), 6.92-7.00 (m, 2H), 6.85 (d, J=7.53 Hz, 1H), 6.69 (dt, J=10.48, 2.16 Hz, 1H), 6.53 (d, J=7.53 Hz, 1H), 6.45 (s, 1H), 4.68 (d, J=7.03 Hz, 1H), 4.30 (d, J=10.79 Hz, 1H), 4.12-4.20 (m, 2H), 3.76-3.90 (m, 4H), 3.55-3.74 (m, 30H), 3.48 (d, J=10.04 Hz, 1H), 3.10-3.26 (m, 2H), 2.82-2.97 (m, 2H), 2.66-2.78 (m, 1H), 2.35-2.50 (m, 2H), 2.22 (s, 3H), 1.65-2.08 (m, 7H), 1.25-1.49 (m, 3H), 0.65 (d, J=7.03 Hz, 3H) LCMS (2 min TFA): Rt=1.04 min, [M/2+H]⁺=557.5.

### 4-((2-(4-((S)-2-((3S,3'S)-3′,6-Dimethylspiro[indoline-3,4′-piperidin]-1′-yl)-1-hydroxyethyl)piperidine-1-carbonyl)-1H-indol-5-yl)oxy)-N-(29-((2,4-dinitrophenyl)amino)-3,6,9,12,15,18,21,24,27-nonaoxanonacosyl)benzamide, 3Trifluoroacetic acid salt

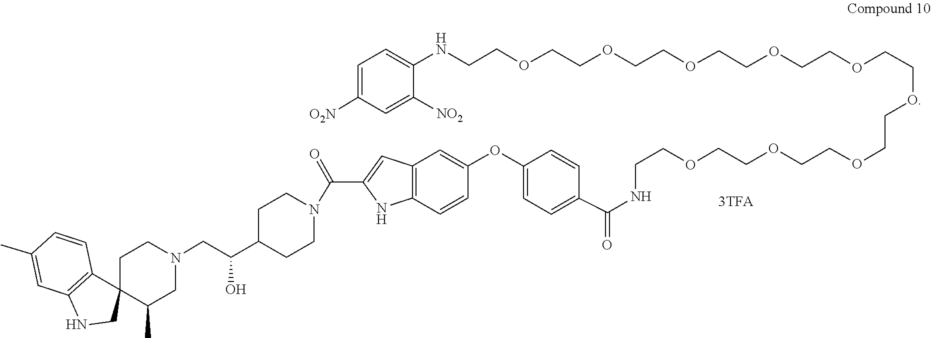 [SMILES: Cc1ccc2c(c1)NC[C@]21CCN(C[C@@H](O)C2CCN(C(=O)c3cc4cc(Oc5ccc(C(=O)NCCOCCOCCOCCOCCOCCOCCOCCOCCOCCNc6ccc([N+](=O)[O-])cc6[N+](=O)[O-])cc5)ccc4[nH]3)CC2)C[C@H]1C]

### Step 1

### Benzyl 3,5-dichloro-4-fluorobenzoate

To a suspension of 3,5-dichloro-4-fluorobenzoic acid (2.0 g, 9.57 mmol) in dichloromethane (DCM) (5.0 mL) was added DMF (0.037 mL, 0.478 mmol) and oxalyl dichloride (1.215 mL, 14.35 mmol) and the mixture was stirred at rt for 1 h. After this period, the mixture was concentrated in vacuo and the residual oxalyl chloride was removed by azeotroping with toluene. The evaporation residue was dissolved in tetrahydrofuran (THF) (50.00 mL) and a solution of triethylamine (4.00 mL, 28.7 mmol) and benzyl alcohol (0.995 mL, 9.57 mmol) in tetrahydrofuran (THF) (50.00 mL) was added rapidly with stirring. The resulting mixture was stirred for 5 min and then concentrated in vacuo. The evaporation residue was partitioned between ether and water, the organic layer was separated, dried over MgSO₄, filtered, and concentrated in vacuo to afford crude benzyl 3,5-dichloro-4-fluorobenzoate (2.71 g, 9.06 mmol, 95% yield) as an orange-brown oil. ¹H NMR (400 MHz, CHLOROFORM-d) □ ppm 8.05 (d, J=6.36 Hz, 2H), 7.38-7.49 (m, 5H). LCMS (2 min TFA): Rt=1.46 min, (compound does not ionize well).

### Step 2

### Ethyl 5-(4-((benzyloxy)carbonyl)-2,6-dichlorophenoxy)-1H-indole-2-carboxylate

To a solution of ethyl 5-hydroxy-1H-indole-2-carboxylate (250 mg, 1.218 mmol) in N-methyl-2-pyrrolidone (NMP) (2.0 mL) was added benzyl 3,5-dichloro-4-fluorobenzoate (364 mg, 1.218 mmol) followed by DIPEA (1.064 mL, 6.09 mmol) and the mixture was heated in a microwave reactor 200° C. 30 min very high then 200 C 6 h very high abs. The crude reaction mixture was partitioned between ether and 1.0 M NaOH, washed with water, dried over magnesium sulfate, and concentrated in vacuo. The evaporation residue was subjected to normal phase purification (0-20% EtOAc/hexanes) to afford ethyl 5-(4-((benzyloxy)carbonyl)-2,6-dichlorophenoxy)-1H-indole-2-carboxylate (234 mg, 0.483 mmol, 39.7% yield) as a pale brown solid. ¹H NMR (400 MHz, CHLOROFORM-d) □ ppm 9.28 (br. s., 1H), 8.13 (s, 2H), 7.37-7.50 (m, 6H), 7.06-7.11 (m, 2H), 6.91 (d, J=2.45 Hz, 1H), 5.41 (s, 2H), 4.43 (q, J=7.09 Hz, 2H), 1.42 (t, J=7.09 Hz, 3H). LCMS (2 min TFA): Rt=1.56 min, [M+H]⁺=484.0.

### Step 3

### 4-((2-(Ethoxycarbonyl)-1H-indol-5-yl)oxy)benzoic acid

To a solution of ethyl 5-(4-((benzyloxy)carbonyl)-2,6-dichlorophenoxy)-1H-indole-2-carboxylate (1.70 g, 3.51 mmol) in tetrahydrofuran (THF) (50.0 mL) and methanol (50.0 mL) was added 10% Pd—C (3.74 g, 3.51 mmol), the flask was purged with nitrogen and then stirred under hydrogen atmosphere for 24 h. After this period, the hydrogen baloon was changed and the mixture was stirred at 40 C for 19 h. Then the mixture was heated to 60° C. for 5 h. The flask was then opened to air, Pd—C was washed down from the walls of the flask, and the heterogeneous mixture was stirred opened to air for 15 min. The catalyst was filtered off by careful gravity filtration and the filtrate was concentrated in vacuo. The crude residue was recrystallized from ethanol to afford 4-((2-(ethoxycarbonyl)-1H-indol-5-yl)oxy)benzoic acid (320 mg, 0.984 mmol, 28.0% yield) as a white solid. The mother liquor was concentrated in vacuo and subjected to acidic reverse phase purification (Waters Sunfire 30×150 mm, MeCN/Water TFA 20-70%) to provide an additional crop of 4-((2-(ethoxycarbonyl)-1H-indol-5-yl)oxy)benzoic acid (155 mg, 0.476 mmol, 13.57% yield). ¹H NMR (400 MHz, DMSO-d₆) □ ppm 12.01 (br. s., 1H), 7.88-7.95 (m, 2H), 7.53 (d, J=8.80 Hz, 1H), 7.41 (d, J=2.45 Hz, 1H), 7.12-7.15 (m, 1H), 7.07 (dd, J=8.80, 2.45 Hz, 1H), 6.94-7.00 (m, 2H), 4.35 (q, J=7.09 Hz, 2H), 1.34 (t, J=7.09 Hz, 3H). LCMS (2 min TFA): Rt=1.01 min, [M+H]⁺=326.2.

### Step 4

### 5-(4-((29-((2,4-Dinitrophenyl)amino)-3,6,9,12,15,18,21,24,27-nonaoxanonacosyl)carbamoyl)phenoxy)-1H-indole-2-carboxylic acid

To a solution of 4-((2-(ethoxycarbonyl)-1H-indol-5-yl)oxy)benzoic acid (95 mg, 0.292 mmol) in N,N-dimethylformamide (DMF) (1.00 mL) was added HATU (111 mg, 0.292 mmol), followed by DIPEA (0.255 mL, 1.460 mmol), and the resulting mixture was stirred at rt for 5 min. After this period, a solution of N1-(2,4-dinitrophenyl)-3,6,9,12,15,18,21,24,27-nonaoxanonacosane-1,29-diamine (200 mg, 0.321 mmol) in N,N-dimethylformamide (DMF) (1.00 mL) was added and the mixture was stirred at rt for 15 minutes before it was quenched by water (0.2 mL) and stirred at rt for 15 min. The mixture was concentrated in vacuo and subjected to normal phase purification on a Biotage Ultra SNAP 50 g silicagel column (0-20% MeOH/DCM).

The above pure ethyl ester was dissolved in a mixture of methanol (1.000 mL) and tetrahydrofuran (THF) (1.000 mL), 1.0 M sodium hydroxide (1.50 mL, 1.500 mmol) was added, and the mixture was stirred at rt for 24 h. After this period, 1.0 M HCl (1.50 mL) was added and the solvents were removed in vacuo. The product was separated from NaCl by extraction with a mixture o DCM/methanol (10:1), solid NaCl was filtered off, and the filtrate concentrated in vacuo to afford 5-(4-((29-((2,4-dinitrophenyl)amino)-3,6,9,12,15,18,21,24,27-nonaoxanonacosyl)carbamoyl)phenoxy)-1H-indole-2-carboxylic acid (260 mg, 0.288 mmol, 99% yield) as a yellow oil. LCMS (2 min TFA): Rt=1.06 min, [M+H]⁺=902.5.

### Step 5

### 4-((2-(4-((S)-2-((3S,3'S)-3′,6-Dimethylspiro[indoline-3,4′-piperidin]-1′-yl)-1-hydroxyethyl)piperidine-1-carbonyl)-1H-indol-5-yl)oxy)-N-(29-((2,4-dinitrophenyl)amino)-3,6,9,12,15,18,21,24,27-nonaoxanonacosyl)benzamide, 3Trifluoroacetic acid salt

To a solution of 5-(4-((29-((2,4-dinitrophenyl)amino)-3,6,9,12,15,18,21,24,27-nonaoxanonacosyl)carbamoyl)phenoxy)-1H-indole-2-carboxylic acid (65 mg, 0.072 mmol) in N,N-dimethylformamide (DMF) (1.00 mL) was added HATU (27.4 mg, 0.072 mmol), followed by DIPEA (0.063 mL, 0.360 mmol), and the resulting mixture was stirred at rt for 5 min. After this period, a solution of (S)-2-((3S,3'S)-3′,6-dimethylspiro[indoline-3,4′-piperidin]-1′-yl)-1-(piperidin-4-yl)ethanol (24.76 mg, 0.072 mmol) in N,N-dimethylformamide (DMF) (1.00 mL) was added and the mixture was stirred at rt for 15 minutes before it was quenched by water (0.2 mL) and stirred at rt for 15 min. The mixture was concentrated in vacuo and was subjected to acidic reverse phase purification (Waters Sunfire 20×100 mm, MeCN+0.1% TFA:Water+0.1% TFA 15-65%) to afford 4-((2-(4-((S)-2-((3S,3'S)-3′,6-dimethylspiro[indoline-3,4′-piperidin]-1′-yl)-1-hydroxyethyl)piperidine-1-carbonyl)-1H-indol-5-yl)oxy)-N-(29-((2,4-dinitrophenyl)amino)-3,6,9,12,15,18,21,24,27-nonaoxanonacosyl)benzamide, 3 trifluoroacetic acid salt (31 mg, 0.020 mmol, 27.4% yield) as a yellow oil. ¹H NMR (400 MHz, METHANOL-d₄) □ ppm 9.02 (d, J=2.76 Hz, 1H), 8.26 (dd, J=9.54, 2.76 Hz, 1H), 7.43 (d, J=15.31 Hz, 1H), 7.07-7.22 (m, 2H), 6.92-7.00 (m, 2H), 6.85 (d, J=7.53 Hz, 1H), 6.69 (dt, J=10.48, 2.16 Hz, 1H), 6.53 (d, J=7.53 Hz, 1H), 6.45 (s, 1H), 4.68 (d, J=7.03 Hz, 1H), 4.30 (d, J=10.79 Hz, 1H), 4.12-4.20 (m, 2H), 3.76-3.90 (m, 4H), 3.55-3.74 (m, 30H), 3.48 (d, J=10.04 Hz, 1H), 3.10-3.26 (m, 2H), 2.82-2.97 (m, 2H), 2.66-2.78 (m, 1H), 2.35-2.50 (m, 2H), 2.22 (s, 3H), 1.65-2.08 (m, 7H), 1.25-1.49 (m, 3H), 0.65 (d, J=7.03 Hz, 3H). LCMS (2 min TFA): Rt=0.97 min, [M/2+H]⁺=614.3.

### 3-(2-(4-((S)-2-((3S,3'S)-3′,6-Dimethylspiro[indoline-3,4′-piperidin]-1′-yl)-1-hydroxyethyl)piperidin-1-yl)oxazol-5-yl)-N-(29-((2,4-dinitrophenyl)amino)-3,6,9,12,15,18,21,24,27-nonaoxanonacosyl)benzamide, 4Trifluoroacetic acid salt

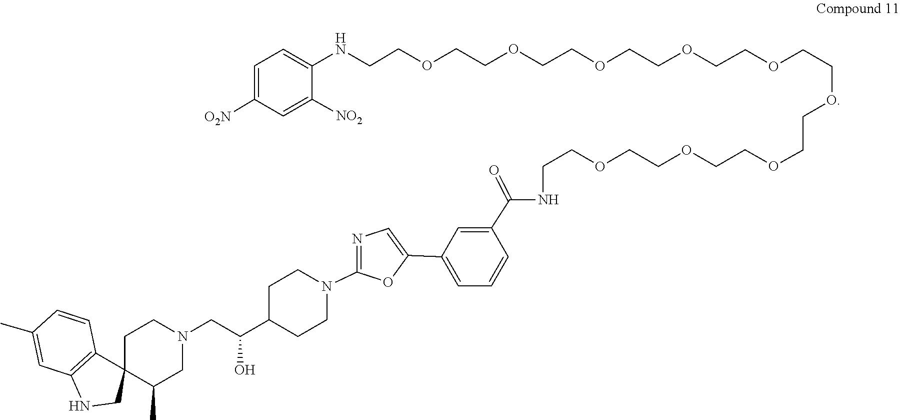 [SMILES: Cc1ccc2c(c1)NC[C@]21CCN(C[C@@H](O)C2CCN(c3ncc(-c4cccc(C(=O)NCCOCCOCCOCCOCCOCCOCCOCCOCCOCCNc5ccc([N+](=O)[O-])cc5[N+](=O)[O-])c4)o3)CC2)C[C@H]1C]

### Step 1

### Methyl 3-(2-chlorooxazol-5-yl)benzoate

A solution of methyl 3-(oxazol-5-yl)benzoate (270 mg, 1.329 mmol) in tetrahydrofuran (THF) (5.0 ml) was cooled to −78° C. and 1.0 M lithium bis(trimethylsilyl)amide in THF (1.462 ml, 1.462 mmol) was added dropwise. After 30 min of stirring, perchloroethane (692 mg, 2.92 mmol) was added in one portion, the flask was re-sealed, purged with nitrogen, taken out of the cooling bath and stirred at ambient temperature for 24 h. After this period, the mixture was quenched with aq. NaH₂PO₄, extracted with EtOAc, the organic layer dried over magnesium sulfate, concentrated in vacuo, and purified on a Biotage Ultra SNAP 50 g silica gel column (0-20% EtAOc/hexanes) to afford methyl 3-(2-chlorooxazol-5-yl)benzoate (297 mg, 1.250 mmol, 94% yield) as a colorless oil which solidified into a white solid upon standing. ¹H NMR (400 MHz, DMSO-d₆) δ ppm 8.16-8.19 (m, 1H), 7.96 (dd, J=7.83, 1.71 Hz, 2H), 7.93 (s, 1H), 7.64 (t, J=7.83 Hz, 1H), 3.89 (s, 3H). LCMS (2 min TFA): Rt=0.97 min, [M+H]⁺=238.2.

### Step 2

### Methyl 3-(2-(4-((S)-2-((3R,3′R)-3′,6-dimethylspiro[indoline-3,4′-piperidin]-1′-yl)-1-hydroxyethyl)piperidin-1-yl)oxazol-5-yl)benzoate

To a solution of (S)-2-((3R,3′R)-3′,6-dimethylspiro[indoline-3,4′-piperidin]-1′-yl)-1-(piperidin-4-yl)ethanol (27 mg, 0.079 mmol) in N,N-dimethylformamide (DMF) (1.00 mL) was added methyl 3-(2-chlorooxazol-5-yl)benzoate (22.41 mg, 0.094 mmol) and DIPEA (0.027 mL, 0.157 mmol) and the mixture was heated in a microwave reactor (160° C., 30 min, very high abs.). After the mixture cooled to rt, the solvents were removed in vacuo, and the evaporation residue subjected to normal phase purification (0-30% MeOH/DCM) to afford methyl 3-(2-(4-((S)-2-((3R,3′R)-3′,6-dimethylspiro[indoline-3,4′-piperidin]-1′-yl)-1-hydroxyethyl)piperidin-1-yl)oxazol-5-yl)benzoate (22 mg, 0.040 mmol, 51.4% yield) as a white solid. ¹H NMR (400 MHz, CHLOROFORM-d) δ ppm 8.13 (s, 1H), 7.86 (d, J=7.83 Hz, 1H), 7.65 (d, J=7.83 Hz, 1H), 7.42 (t, J=7.83 Hz, 1H), 7.14 (s, 1H), 6.91 (d, J=7.58 Hz, 1H), 6.57 (d, J=7.34 Hz, 1H), 6.45 (s, 1H), 4.18-4.32 (m, 2H), 3.95 (s, 3H), 3.58-3.66 (m, 1H), 3.54 (d, J=9.54 Hz, 1H), 3.29 (d, J=9.78 Hz, 1H), 2.86-3.09 (m, 4H), 2.36-2.58 (m, 3H), 2.27 (s, 3H), 2.00-2.16 (m, 3H), 1.68-1.93 (m, 3H), 1.43-1.65 (m, 3H), 0.69 (d, J=6.85 Hz, 3H). LCMS (2 min TFA): Rt=0.71 min, [M+H]⁺=545.2

### Step 3

### 3-(2-(4-((S)-2-((3S,3'S)-3′,6-Dimethylspiro[indoline-3,4′-piperidin]-1′-yl)-1-hydroxyethyl)piperidin-1-yl)oxazol-5-yl)-N-(29-((2,4-dinitrophenyl)amino)-3,6,9,12,15,18,21,24,27-nonaoxanonacosyl)benzamide, 4Trifluoroacetic acid salt

To a solution of methyl 3-(2-(4-((S)-2-((3S,3'S)-3′,6-dimethylspiro[indoline-3,4′-piperidin]-1′-yl)-1-hydroxyethyl)piperidin-1-yl)oxazol-5-yl)benzoate (22 mg, 0.040 mmol) in methanol (2.00 mL) was added 1.0 M sodium hydroxide (0.404 mL, 0.404 mmol) and the resulting solution was heated in a microwave reactor (65° C., 30 min, very high asb.). After the mixture cooled down to rt, 1.0 M HCl (0.404 mL) was added and the solvents were removed in vacuo (0 mbar, 37° C., 1 h) and the residual water was removed by azeotroping with PhMe. The evaporation residue was dissolved in N,N-dimethylformamide (DMF) (1.000 mL), HATU (16.89 mg, 0.044 mmol) and DIPEA (0.071 mL, 0.404 mmol) were added, and the mixture was stirred at rt for 5 min before a solution of N1-(2,4-dinitrophenyl)-3,6,9,12,15,18,21,24,27-nonaoxanonacosane-1,29-diamine (30 mg, 0.048 mmol) in N,N-dimethylformamide (DMF) (1.000 mL) was added and the stirring continued for 1 h. After this period, water (0.05 mL) was added and the mixture was subjected to acidic reverse phase purification (Waters Sunfire 30×100 mm, MeOH+0.1% FA/Water+0.1% FA 10-60%) to afford 3-(2-(4-((S)-2-((3S,3'S)-3′,6-dimethylspiro[indoline-3,4′-piperidin]-1′-yl)-1-hydroxyethyl)piperidin-1-yl)oxazol-5-yl)-N-(29-((2,4-dinitrophenyl)amino)-3,6,9,12,15,18,21,24,27-nonaoxanonacosyl)benzamide, 4 trifluoroacetic acid salt (9.9 mg, 6.22 μmol, 15.40% yield) as a yellow oil. ¹H NMR (400 MHz, METHANOL-d₄) δ ppm 8.99 (d, J=2.69 Hz, 1H), 8.25 (dd, J=9.54, 2.69 Hz, 1H), 8.05 (t, J=1.59 Hz, 1H), 7.71-7.84 (m, 2H), 7.59 (s, 1H), 7.53 (t, J=7.95 Hz, 1H), 7.23 (s, 2H), 7.18 (d, J=9.54 Hz, 1H), 7.14 (s, 1H), 4.24 (br. s., 2H), 3.84-3.99 (m, 2H), 3.77-3.82 (m, 2H), 3.52-3.75 (m, 40H), 3.21-3.29 (m, 3H), 2.36-2.48 (m, 4H), 2.05 (d, J=13.94 Hz, 2H), 1.72-1.90 (m, 2H), 1.51-1.68 (m, 2H), 0.77 (d, J=6.85 Hz, 3H). LCMS (2 min TFA): Rt=0.94 min, [M/2+H]⁺=568.8.

### 4-((2-(4-((S)-2-((3S,3'S)-3′,6-Dimethylspiro[indoline-3,4′-piperidin]-1′-yl)-1-hydroxyethyl)piperidine-1-carbonyl)-1H-indol-6-yl)oxy)-N-(29-((2,4-dinitrophenyl)amino)-3,6,9,12,15,18,21,24,27-nonaoxanonacosyl)benzamide, 3Trifluoroacetic acid salt

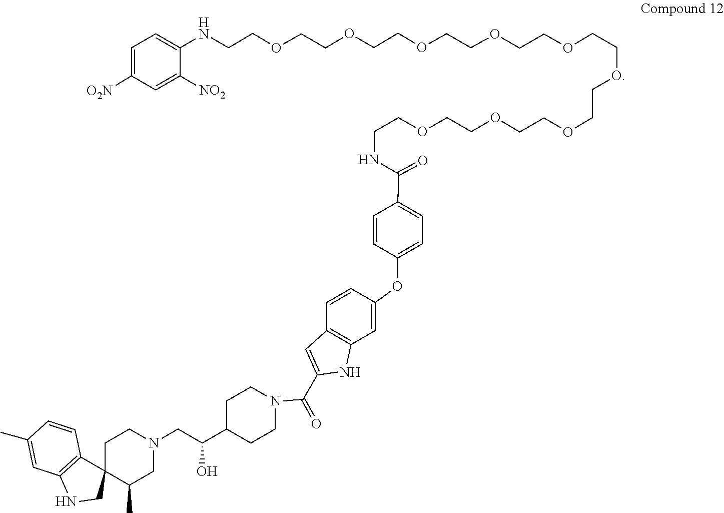 [SMILES: Cc1ccc2c(c1)NC[C@]21CCN(C[C@@H](O)C2CCN(C(=O)c3cc4ccc(Oc5ccc(C(=O)NCCOCCOCCOCCOCCOCCOCCOCCOCCOCCNc6ccc([N+](=O)[O-])cc6[N+](=O)[O-])cc5)cc4[nH]3)CC2)C[C@H]1C]

### Step 1

### Methyl 6-(4-((benzyloxy)carbonyl)-2,6-dichlorophenoxy)-1H-indole-2-carboxylate

To a 20 mL microwave vial was added methyl 6-hydroxy-1H-indole-2-carboxylate (1.2876 g, 6.73 mmol) as a suspension in N-methyl-2-pyrrolidone (NMP) (6.91 ml). To this suspension, benzyl 3,5-dichloro-4-fluorobenzoate (1.3425 g, 4.49 mmol) and N-ethyl-N-isopropylpropan-2-amine (3.14 ml, 17.95 mmol) were added at which point the solids went into solution. The vial was sealed and placed into a microwave reactor at 200° C., very high abs. for 6 h. The crude reaction mixture was partitioned between ether and 1.0 M NaOH, the ether layer was separated, dried over MgSO₄, filtered, concentrated in vacuo, and purified on a Biotage Ultra SNAP column (6-50% EtOAc/hexanes) to afford methyl 6-(4-((benzyloxy)carbonyl)-2,6-dichlorophenoxy)-1H-indole-2-carboxylate (0.6826 g, 1.379 mmol, 30.7% yield) as a white crystalline solid. ¹H NMR (400 MHz, CHLOROFORM-d) δ ppm 8.69 (br. s., 1H), 8.12 (s, 2H), 7.63 (d, J=8.56 Hz, 1H), 7.36-7.51 (m, 5H), 7.19 (s, 1H), 6.85 (dd, J=8.80, 2.20 Hz, 1H), 6.69 (s, 1H), 5.40 (s, 2H), 3.92 (s, 3H). LCMS (2 min TFA): Rt=1.52 min, [M+H]⁺=470.2.

### Step 2

### 4-((2-(methoxycarbonyl)-1H-Indol-6-yl)oxy)benzoic acid

To a solution of methyl 6-(4-((benzyloxy)carbonyl)-2,6-dichlorophenoxy)-1H-indole-2-carboxylate (680 mg, 1.446 mmol) in tetrahydrofuran (THF) (25.0 mL) and methanol (25.0 mL) was added 10% Pd—C (3077 mg, 2.89 mmol), the flask was purged with nitrogen and then stirred under hydrogen atmosphere for 48 h. After this period, the heterogeneous mixture was stirred opened to air for 15 min. The catalyst was filtered off by careful gravity filtration and the filtrate was concentrated in vacuo. The crude residue was recrystallized from methanol/DCM by slow evaporation on the rotavap (37 C, 350 mbar) and air-dried to afford 4-((2-(methoxycarbonyl)-1H-indol-6-yl)oxy)benzoic acid (173 mg, 0.556 mmol, 38.4% yield) as a grey powder. ¹H NMR (400 MHz, DMSO-d₆) δ ppm 12.88 (br. s., 1H), 11.96 (s, 1H), 7.94 (d, J=8.78 Hz, 2H), 7.73 (d, J=8.78 Hz, 1H), 7.19 (d, J=1.25 Hz, 1H), 7.01-7.12 (m, 3H), 6.90 (dd, J=8.66, 2.13 Hz, 1H). LCMS (2 min TFA): Rt=0.97 min, [M+H]⁺=312.1.

### Step 3

### 6-(4-((29-((2,4-Dinitrophenyl)amino)-3,6,9,12,15,18,21,24,27-nonaoxanonacosyl)carbamoyl)phenoxy)-1H-indole-2-carboxylic acid

To a solution of 4-((2-(methoxycarbonyl)-1H-indol-6-yl)oxy)benzoic acid (146 mg, 0.469 mmol) in N,N-dimethylformamide (DMF) (1.50 mL) was added HATU (178 mg, 0.469 mmol), followed by DIPEA (0.410 mL, 2.345 mmol), and the resulting mixture was stirred at rt for 1 min. After this period, a solution of N1-(2,4-dinitrophenyl)-3,6,9,12,15,18,21,24,27-nonaoxanonacosane-1,29-diamine (292 mg, 0.469 mmol) in N,N-dimethylformamide (DMF) (1.50 mL) was added and the mixture was stirred at rt for 2 h before it was quenched by water (0.2 mL) and stirred at rt for an additional 15 min. The crude reaction mixture was partitioned between ethyl acetate, and a 3:3:1 mixture of water, brine, and 26% aqueous ammonia. The organic layer was separated, dried over sodium sulfate, filtered, and concentrated in vacuo to afford the crude intermediate methyl ester.

The above methyl ester was dissolved in a mixture of methanol (5.00 mL) and tetrahydrofuran (THF) (5.00 mL), 1.0 M sodium hydroxide (3.00 mL, 3.00 mmol) was added, and the mixture was stirred at rt for 16 h. After this period, the mixture was concentrated in vacuo, the evaporation residue dissolved in water (75 mL), filtered, the filtrate acidified by 1.0 M HCl (3.0 mL) and extracted with a 10:1 mixture of DCM/IPA. The organic layer was separated, dried over sodium sulfate, filtered, and concentrated in vacuo to afford 6-(4-((29-((2,4-dinitrophenyl)amino)-3,6,9,12,15,18,21,24,27-nonaoxanonacosyl)carbamoyl)phenoxy)-1H-indole-2-carboxylic acid (344 mg, 0.381 mmol, 81% yield) as a thick red oil. LCMS (2 min TFA): Rt=1.09 min, [M+H]⁺=902.1.

### Step 4

### 4-((2-(4-((S)-2-((3S,3'S)-3′,6-Dimethylspiro[indoline-3,4′-piperidin]-1′-yl)-1-hydroxyethyl)piperidine-1-carbonyl)-1H-indol-6-yl)oxy)-N-(29-((2,4-dinitrophenyl)amino)-3,6,9,12,15,18,21,24,27-nonaoxanonacosyl)benzamide, 3Trifluoroacetic acid salt

To a solution of 6-(4-((29-((2,4-dinitrophenyl)amino)-3,6,9,12,15,18,21,24,27-nonaoxanonacosyl)carbamoyl)phenoxy)-1H-indole-2-carboxylic acid (86 mg, 0.095 mmol) in N,N-dimethylformamide (DMF) (1.00 mL) was added HATU (36.3 mg, 0.095 mmol), followed by DIPEA (0.083 mL, 0.477 mmol), and the resulting mixture was stirred at rt for 30 seconds. After this period, a solution of (S)-2-((3S,3'S)-3′,6-dimethylspiro[indoline-3,4′-piperidin]-1′-yl)-1-(piperidin-4-yl)ethanol (32.8 mg, 0.095 mmol) in N,N-dimethylformamide (DMF) (1.00 mL) was added and the mixture was stirred at rt for 1 hour before it was quenched by water (2 drops) and subjected to reverse phase purification (Waters Sunfire 30×150 mm Acetonitrile:Water TFA 10-65%) to afford 4-((2-(4-((S)-2-((3S,3'S)-3′,6-dimethylspiro[indoline-3,4′-piperidin]-1′-yl)-1-hydroxyethyl)piperidine-1-carbonyl)-1H-indol-6-yl)oxy)-N-(29-((2,4-dinitrophenyl)amino)-3,6,9,12,15,18,21,24,27-nonaoxanonacosyl)benzamide, 3 trifluoroacetic acid salt (52 mg, 0.033 mmol, 34.7% yield) as a yellow oil. ¹H NMR (400 MHz, METHANOL-d₄) □ ppm 8.94 (d, J=2.76 Hz, 1H), 8.20 (dd, J=9.54, 2.76 Hz, 1H), 7.81 (d, J=8.78 Hz, 2H), 7.62 (d, J=8.78 Hz, 1H), 7.34-7.39 (m, 1H), 7.27-7.32 (m, 2H), 7.08-7.16 (m, 2H), 6.99 (d, J=8.78 Hz, 2H), 6.79-6.87 (m, 2H), 4.67 (br. s., 2H), 3.93 (d, J=12.55 Hz, 2H), 3.73-3.82 (m, 4H), 3.53-3.68 (m, 34H), 2.46-2.55 (m, 1H), 2.42 (s, 3H), 1.94-2.11 (m, 2H), 1.71-1.87 (m, 2H), 1.46 (br. s, 2H), 0.76 (d, J=7.03 Hz, 3H). LCMS (2 min TFA): Rt=1.02 min, [M/2+H]⁺=614.6.

### N1-(2-(2-(2-(2-((2-(4-((S)-2-((3S,3'S)-3′,6-Dimethylspiro[indoline-3,4′-piperidin]-1′-yl)-1-hydroxyethyl)piperidine-1-carbonyl)-1H-indol-6-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide trifluoroacetic acid salt

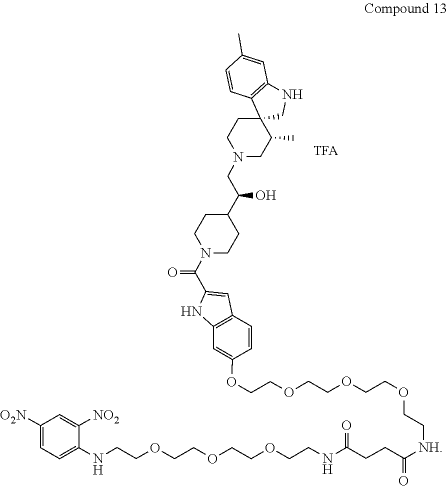 [SMILES: Cc1ccc2c(c1)NC[C@]21CCN(C[C@@H](O)C2CCN(C(=O)c3cc4ccc(OCCOCCOCCOCCNC(=O)CCC(=O)NCCOCCOCCOCCNc5ccc([N+](=O)[O-])cc5[N+](=O)[O-])cc4[nH]3)CC2)C[C@H]1C]

### Step 1

### Methyl 6-((tert-butyldimethylsilyl)oxy)-1H-indole-2-carboxylate

To a stirred solution of methyl 6-hydroxy-1H-indole-2-carboxylate (502.5 mg, 2.52 mmol) in Dichloromethane (DCM) (15 mL) was added imidazole (694 mg, 10.09 mmol), followed by addition of TBS-Cl (881 mg, 5.55 mmol) in portions. The reaction mixture was stirred at room temperature for 2 h. The reaction mixture was diluted with DCM, washed with water twice and brine, and dried over Na₂SO₄, filtered, and concentrated to give a light brown solid, which was purified by silica gel chromatography (0-100% Hexanes/DCM) to give the title compound (742 mg, 95% yield) as a white solid. ¹H NMR (400 MHz, DMSO-d₆) δ ppm 11.61 (br. s., 1H) 7.52 (d, J=8.80 Hz, 1H) 7.09 (d, J=1.22 Hz, 1H) 6.86 (d, J=1.96 Hz, 1H) 6.67 (dd, J=8.56, 2.20 Hz, 1H) 3.85 (s, 3H) 0.97 (s, 9H) 0.20 (s, 6H). LC-MS: m/z 306 (M+1).

### Step 2

### 1-Tert-butyl 2-methyl 6-((tert-butyldimethylsilyl)oxy)-1H-indole-1,2-dicarboxylate

To a stirred solution of methyl 6-((tert-butyldimethylsilyl)oxy)-1H-indole-2-carboxylate (739 mg, 2.395 mmol) in Dichloromethane (DCM) (10 mL) was added Boc-anhydride (634 mg, 2.87 mmol), followed by addition of DMAP (29.3 mg, 0.240 mmol). The reaction mixture was stirred at room temperature for 45 minutes. The reaction mixture was purified by silica gel chromatography (0-10% Hexanes/EtOAc) to give the title compound (1000.8 mg, 97% pure, 100% yield) as a colorless viscous oil. ¹H NMR (400 MHz, CHLOROFORM-d) δ ppm 7.58 (d, J=2.20 Hz, 1H) 7.45 (d, J=8.56 Hz, 1H) 7.08 (s, 1H) 6.83 (dd, J=8.56, 2.20 Hz, 1H) 3.92 (s, 3H) 1.64 (s, 9H) 1.03 (s, 9H) 0.25 (s, 6H). LC-MS: m/z 350 (M+1-isobutene).

### Step 3

### 1-Tert-butyl 2-methyl 6-hydroxy-1H-indole-1,2-dicarboxylate

To a stirred solution of 1-tert-butyl 2-methyl 6-((tert-butyldimethylsilyl)oxy)-1H-indole-1,2-dicarboxylate (998 mg, 97% pure, 2.387 mmol) in Tetrahydrofuran (THF) (15 mL) cooled to 0° C. in an ice bath was added TBAF (1.0 M in THF) (2.98 mL, 2.98 mmol) dropwise. The reaction mixture was stirred at 0° C. for 1 h. Diluted with EtOAc, washed with saturated aqueous NH₄Cl, brine, dried over Na₂SO₄, filtered, and concentrated to a brown oil, which was purified by silica gel chromatography (0-25% Hexanes/EtOAc) to give the title compound (654 mg, 94% yield) as a white solid. ¹H NMR (400 MHz, CHLOROFORM-d) δ ppm 7.60 (d, J=2.45 Hz, 1H) 7.48 (d, J=8.56 Hz, 1H) 7.09 (d, J=0.73 Hz, 1H) 6.85 (dd, J=8.44, 2.32 Hz, 1H) 4.89 (br. s., 1H) 3.92 (s, 3H) 1.63 (s, 9H). LC-MS: m/z 236 (M+1-isobutene).

### Step 4

### 28-((2,4-Dinitrophenyl)amino)-13,16-dioxo-3,6,9,20,23,26-hexaoxa-12,17-diazaoctacosyl 4-methylbenzenesulfonate

To a stirred solution of N1-(2-(2-(2-(2-((2,4-dinitrophenyl)amino)ethoxy)ethoxy)ethoxy)ethyl)-N4-(2-(2-(2-(2-hydroxyethoxy)ethoxy)ethoxy)ethyl)succinamide (125 mg, 0.185 mmol) in Dichloromethane (DCM) (2 mL) cooled to 0° C. in an ice bath was added Et₃N (0.078 mL, 0.556 mmol), DMAP (2.265 mg, 0.019 mmol), followed by Tosyl-Cl (37.1 mg, 0.195 mmol). The reaction mixture was stirred at 0° C. for 1 h, then allowed to warm up to room temperature and stirred overnight. Diluted with DCM, washed with saturated aqueous NaH₂PO₄, then saturated aqueous NaHCO₃, brine, dried over Na₂SO₄, filtered, and concentrated to a yellow oil, which was purified by silica gel chromatography (0-15% DCM/MeOH) to give the title compound (128.8 mg, 88% yield) as a yellow viscous oil. ¹H NMR (400 MHz, METHANOL-d₄) δ ppm 8.96-9.18 (m, 1H) 8.22-8.38 (m, 1H) 7.81 (d, J=8.31 Hz, 2H) 7.46 (d, J=8.07 Hz, 2H) 7.24 (d, J=9.54 Hz, 1H) 4.13-4.22 (m, 2H) 3.79-3.87 (m, 2H) 3.51-3.75 (m, 26H) 2.39-2.53 (m, 9H). LC-MS: m/z 788 (M+1).

### Step 5

### 1-Tert-butyl 2-methyl 6-((28-((2,4-dinitrophenyl)amino)-13,16-dioxo-3,6,9,20,23,26-hexaoxa-12,17-diazaoctacosyl)oxy)-1H-indole-1,2-dicarboxylate

To a stirred solution of 28-((2,4-dinitrophenyl)amino)-13,16-dioxo-3,6,9,20,23,26-hexaoxa-12,17-diazaoctacosyl 4-methylbenzenesulfonate (127.8 mg, 0.162 mmol) in N,N-Dimethylformamide (DMF) (2 mL) was added 1-tert-butyl 2-methyl 6-hydroxy-1H-indole-1,2-dicarboxylate (59.1 mg, 0.203 mmol) followed by cesium carbonate (211 mg, 0.649 mmol). The reaction mixture was stirred at room temperature overnight. Most of DMF was removed by N₂ flow at room temperature. Water was added, extracted with EtOAc twice. The combined organic extract was washed with water three times, brine, dried over Na₂SO₄, filtered, and concentrated to a yellow viscous oil, which was purified by silica gel chromatography (0-15% DCM/MeOH) to give the title compound (139 mg, 94% yield) as a yellow viscous oil. ¹H NMR (400 MHz, METHANOL-d₄) δ ppm 9.00 (d, J=2.69 Hz, 1H) 8.15-8.34 (m, 1H) 7.59 (d, J=2.20 Hz, 1H) 7.52 (d, J=8.80 Hz, 1H) 7.17 (d, J=9.78 Hz, 1H) 7.11 (s, 1H) 6.94 (dd, J=8.80, 2.20 Hz, 1H) 4.18-4.26 (m, 2H) 3.87-3.94 (m, 5H) 3.78-3.84 (m, 2H) 3.73-3.78 (m, 2H) 3.56-3.73 (m, 17H) 3.47-3.56 (m, 5H) 2.47 (s, 4H) 1.63 (s, 9H). LC-MS: m/z 907 (M+1).

### Step 6

### 6-((28-((2,4-Dinitrophenyl)amino)-13,16-dioxo-3,6,9,20,23,26-hexaoxa-12,17-diazaoctacosyl)oxy)-1H-indole-2-carboxylic acid

To a stirred solution of 1-tert-butyl 2-methyl 6-((28-((2,4-dinitrophenyl)amino)-13,16-dioxo-3,6,9,20,23,26-hexaoxa-12,17-diazaoctacosyl)oxy)-1H-indole-1,2-dicarboxylate (138 mg, 0.152 mmol) in Dichloromethane (DCM) (2 mL) was added 4M HCl in 1,4-dioxane (0.304 mL, 1.217 mmol). The reaction mixture was stirred at room temperature overnight. More 4M HCl in 1,4-dioxane (0.152 mL, 0.609 mmol) was added. Stirred at room temperature for another 4 h. The solvent was removed under vacuo to give a yellow oil, which was redissolved in Methanol (2.25 mL). 1N NaOH (1 mL, 1.000 mmol) was added, and the mixture was stirred at room temperature overnight. 1 mL of 1N HCl (1 mL, 1.000 mmol) was added, and the mixture was concentrated under N₂ flow. MeOH was added to dissolve the crude product, and the solution was purified on Gilson reverse phase Prep HPLC (15% CH₃CN in water to 55% CH₃CN in water with 0.1% TFA). The fractions containing the product were combined and concentrated on rotavap (water bath temperature: 26° C.). The yellow oily residue was transferred to a vial using CH₃CN, and concentrated using N₂ flow, then lyophilized (with 1:1 CH₃CN and water) to the title compound (83.1 mg, 68.9% yield) as an orange gum. ¹H NMR (400 MHz, METHANOL-d₄) δ ppm 9.01 (d, J=2.69 Hz, 1H) 8.26 (dd, J=9.54, 2.69 Hz, 1H) 7.49 (d, J=8.80 Hz, 1H) 7.17 (d, J=9.78 Hz, 1H) 7.08 (d, J=0.73 Hz, 1H) 6.93 (d, J=1.96 Hz, 1H) 6.76 (dd, J=8.80, 2.20 Hz, 1H) 4.18 (dd, J=5.38, 3.91 Hz, 2H) 3.90 (dd, J=5.38, 3.91 Hz, 2H) 3.45-3.83 (m, 24H) 2.47 (s, 4H). LC-MS: m/z 793 (M+1).

### Step 7

### N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-6-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide trifluoroacetic acid salt

To a stirred solution of 6-((28-((2,4-dinitrophenyl)amino)-13,16-dioxo-3,6,9,20,23,26-hexaoxa-12,17-diazaoctacosyl)oxy)-1H-indole-2-carboxylic acid (63.9 mg, 0.081 mmol) in N,N-Dimethylformamide (DMF) (1 mL) was added HATU (31.6 mg, 0.081 mmol) followed by DIPEA (0.056 mL, 0.322 mmol). The resulting mixture was stirred at room temperature for 5 minutes, then a solution of (S)-2-((3S,3'S)-3′,6-dimethylspiro[indoline-3,4′-piperidin]-1′-yl)-1-(piperidin-4-yl)ethanol (27.7 mg, 0.081 mmol) in N,N-Dimethylformamide (DMF) (1 mL) was added dropwise. The reaction mixture was stirred at room temperature for 20 minutes. The solution was then purified on Gilson reverse phase Prep HPLC (15% CH3CN in water to 50% CH3CN in water over with 0.1% TFA). The fractions containing the product were combined and concentrated on rotavap (water bath temperature: 30° C.). The yellow oily residue was transferred to a vial using CH₃CN, and concentrated using N₂ flow, then lyophilized (with 1:1 CH₃CN and water) to give the title compound (69.7 mg, 70.2% yield) as a yellow solid. ¹H NMR (400 MHz, METHANOL-d₄) δ ppm 9.00 (d, J=2.69 Hz, 1H) 8.25 (dd, J=9.54, 2.69 Hz, 1H) 7.47 (d, J=8.80 Hz, 1H) 7.17 (d, J=9.78 Hz, 1H) 7.08 (d, J=7.58 Hz, 1H) 6.88-7.00 (m, 2H) 6.83 (s, 1H) 6.69-6.79 (m, 2H) 4.73 (d, J=11.25 Hz, 2H) 4.11-4.23 (m, 2H) 3.45-3.99 (m, 33H) 2.98-3.14 (m, 2H) 2.81-2.94 (m, 1H) 2.47 (s, 4H) 2.21-2.41 (m, 5H) 1.92-2.09 (m, 2H) 1.79 (d, J=11.74 Hz, 2H) 1.36-1.57 (m, 2H) 0.80 (d, J=6.85 Hz, 3H). LC-MS: m/z 1118 (M+1).

### N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-4-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide trifluoroacetic acid salt

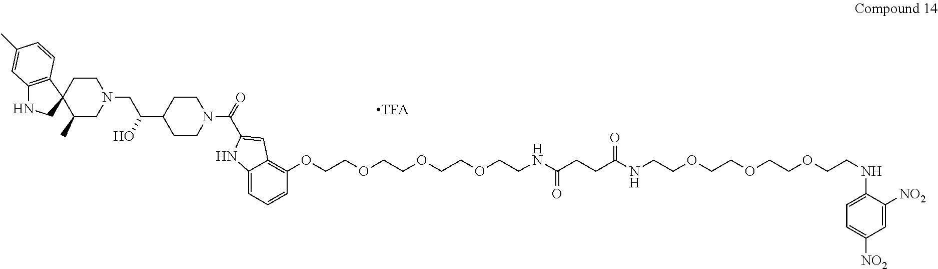 [SMILES: Cc1ccc2c(c1)NC[C@]21CCN(C[C@@H](O)C2CCN(C(=O)c3cc4c(OCCOCCOCCOCCNC(=O)CCC(=O)NCCOCCOCCOCCNc5ccc([N+](=O)[O-])cc5[N+](=O)[O-])cccc4[nH]3)CC2)C[C@H]1C]

### Step 1

### 1-Tert-butyl 2-ethyl 4-(benzyloxy)-1H-indole-1,2-dicarboxylate

Ethyl 4-(benzyloxy)-1H-indole-2-carboxylate (255 mg, 0.820 mmol) and DMAP (10.02 mg, 0.082 mmol) were suspended in dichloromethane (1.75 mL). A solution of boc-anhydride (0.209 mL, 0.902 mmol) in dichloromethane (1.75 mL) was added drop wise and the mixture was stirred for 2 hours at room temperature. The solvent was evaporated and the residue was purified by silica gel chromatography (0-15% Hexanes/EtOAc) to give 1-tert-butyl 2-ethyl 4-(benzyloxy)-1H-indole-1,2-dicarboxylate (334 ng, 0.819 mmol, 100% yield) as a colorless oil.

LCMS: m/z (M+Na=418)

¹H NMR (400 MHz, DICHLOROMETHANE-d₂) δ ppm 7.68 (d, J=8.56 Hz, 1H) 7.49-7.58 (m, 2H) 7.28-7.48 (m, 5H) 6.80 (d, J=8.07 Hz, 1H) 4.38 (q, J=7.09 Hz, 2H) 1.67 (s, 9H) 1.56 (s, 2H) 1.41 (t, J=7.21 Hz, 3H)

### Step 2

### 1-Tert-butyl 2-ethyl 4-hydroxy-1H-indole-1,2-dicarboxylate

10% palladium on carbon (10 mg, 9.40 μmol) was placed under a N₂ atmosphere into a round-bottom flask and covered with ethanol (0.5 mL). A solution of 1-tert-butyl 2-ethyl 4-(benzyloxy)-1H-indole-1,2-dicarboxylate (1) (334 mg, 0.845 mmol) in ethanol (2.500 nL) was added, followed by ammonium formate (60.4 mg, 0.929 mmol). The mixture was stirred for 30 minutes at room temperature, no conversion was seen by LCMS. Another portion of 10% Pd—C (90 mg, 0.085 mmol) was added to the reaction mixture and let stirred at room temperature for another 30 minutes. Acetone (2.000 mL) was added and the reaction was checked after 2.5 hours; no change was seen by LCMS. A H₂ balloon was attached to the flask and the reaction was stirred at room temperature. After 1 hour the mixture was filtered on celite, washed with ethanol, and then concentrated under vacuo. The residue was dissolved in EtOAc and was washed with water, brine, dried over Na₂SO₄, filtered, and concentrated. The residue purified by silica gel chromatography (0 to 5% DCM/EtOAc) to give 1-tert-butyl 2-ethyl 4-hydroxy-1H-indole-1,2-dicarboxylate (233.5 mg, 0.757 mmol, 92% yield) as a white solid.

LCMS: m/z (M+Na=328)

¹H-NMR (DMSO-d₆): δ (ppm) 10.2 (br, 1H), 7.4 (d, 1H), 7.28 (s, 1H), 7.25 (dd, 1H), 6.67 (d, 1H), 4.3 (q, 2H), 1.53 (s, 9H), 1.3 (t, 3H).

### Step 3

### 28-((2,4-Dinitrophenyl)amino)-13,16-dioxo-3,6,9,20,23,26-hexaoxa-12,17-diazaoctacosyl 4-methylbenzenesulfonate

To a solution of N1-(2-(2-(2-(2-((2,4-dinitrophenyl)amino)ethoxy)ethoxy)ethoxy)ethyl)-N4-(2-(2-(2-(2-hydroxyethoxy)ethoxy)ethoxy)ethyl)succinamide (150 mg, 0.223 mmol) in dichloromethane (2.5 mL) in an ice bath was added TEA (0.093 mL, 0.668 mmol) followed by DMAP (2.72 mg, 0.022 mmol) and 4-methylbenzene-1-sulfonyl chloride (44.5 mg, 0.234 mmol). The solution was allowed to warm up and stir at room temperature over night. It was then diluted with DCM. NaH₂PO₄ was added (10 mL), the mixture was extracted with DCM. The combined organic extracts were washed with sat'd aqueous NaHCO₃, brine, dried over Na₂SO₄, filtered and concentrated to give a yellow oil, which was purified by silica gel chromatography (0 to 15% DCM/MeOH), to give 28-((2,4-dinitrophenyl)amino)-13,16-dioxo-3,6,9,20,23,26-hexaoxa-12,17-diazaoctacosyl 4-methylbenzenesulfonate (176 ng, 0.219 mmol, 98% yield) as a yellow oil.

LCMS: m/z (M+1=788)

¹H NMR (400 MHz, CHLOROFORM-d) δ ppm 9.16 (d, J=2.45 Hz, 1H) 8.83 (br. s., 1H) 8.29 (dd, J=9.54, 2.69 Hz, 1H) 7.82 (d, J=8.31 Hz, 2H) 7.37 (d, J=8.07 Hz, 2H) 6.99 (d, J=9.78 Hz, 1H) 6.65 (br. s., 1H) 4.15-4.21 (m, 2H) 3.87 (t, J=5.26 Hz, 2H) 3.53-3.79 (m, 24H) 3.46 (d, J=3.18 Hz, 4H) 2.59 (br. s., 4H) 2.47 (s, 3H).

### Step 4

### 1-Tert-butyl 2-ethyl 4-((28-((2,4-dinitrophenyl)amino)-13,16-dioxo-3,6,9,20,23,26-hexaoxa-12,17-diazaoctacosyl)oxy)-1H-indole-1,2-dicarboxylate

To the solution of 28-((2,4-dinitrophenyl)amino)-13,16-dioxo-3,6,9,20,23,26-hexaoxa-12,17-diazaoctacosyl 4-methylbenzenesulfonate (175 mg, 0.220 mmol) in N,N-Dimethylformamide (DMF) (2 mL) was added 1-tert-butyl 2-ethyl 4-hydroxy-1H-indole-1,2-dicarboxylate (2) (85 mg, 0.275 mmol) and Cs₂CO₃ (287 mg, 0.880 mmol) and the red solution was stirred overnight. The DMF was evaporated under a nitrogen flow. Water was added and the reaction was extracted three times (25 mL, 25 mL, 10 mL) with EtOAc, the combined organic extracts were washed with water, then brine, dried over Na₂SO₄, filtered, and concentrated. The concentrate purified by silica gel chromatography (0 to 25% DCM/MeOH), and purified to give a yellow oil which was 84% pure. The concentrate was once more purified by silica gel chromatography (0 to 15% DCM/MeOH) to give 1-tert-butyl 2-ethyl 4-((28-((2,4-dinitrophenyl)amino)-13,16-dioxo-3,6,9,20,23,26-hexaoxa-12,17-diazaoctacosyl)oxy)-1H-indole-1,2-dicarboxylate (171 mg, 0.184 mmol, 83% yield) as a yellow oil.

LCMS: m/z (M+Na=943)

¹H NMR (400 MHz, METHANOL-d₄) δ ppm 8.97 (d, 1=2.69 Hz, 1H) 8.22 (dd, J=9.54, 2.69 Hz, 1H) 7.55 (d, J=8.56 Hz, 1H) 7.32 (t, J=8.19 Hz, 1H) 7.19 (d, J=0.73 Hz, 1H) 7.13 (d, J=9.78 Hz, 1H) 6.76 (d, J=7.82 Hz, 1H) 4.36 (q, J=7.17 Hz, 2H) 4.23-4.30 (m, 2H) 3.93 (dd, J=5.26, 3.79 Hz, 2H) 3.43-3.82 (m, 26H) 2.44 (s, 4H) 1.62 (s, 9H) 1.39 (t, J=7.09 Hz, 3H)

### Step 5

### 4-((28-((2,4-dinitrophenyl)amino)-13,16-dioxo-3,6,9,20,23,26-hexaoxa-12,17-diazaoctacosyl)oxy)-1H-indole-2-carboxylic acid

To a solution of 1-tert-butyl 2-ethyl 4-((28-((2,4-dinitrophenyl)amino)-13,16-dioxo-3,6,9,20,23,26-hexaoxa-12,17-diazaoctacosyl)oxy)-1H-indole-1,2-dicarboxylate (170 mg, 0.185 mmol) in dichloromethane (DCM) (2.5 mL) under a nitrogen flow was added HCl in dioxane (0.369 mL, 1.477 mmol) and the solution was stirred at room temperature over night. 9% starting material left over was seen by LCMS. Another portion of HCl in dioxane (0.185 mL, 0.738 mmol) was added and the reaction was stirred for a total of 22 h when the solvent was evaporated by blowing down nitrogen on it. The crude product was taken up to the next step without any further purification.

LCMS: m/z (M+H=821)

The crude product was dissolved in methanol (2.5 mL) and 1 M NaOH (1.018 mL, 1.018 mmol) was added to this solution. The reaction mixture was stirred overnight. After 16 h, 1 mL of 1 M HCl was added and the solvent was evaporated by blowing down N₂ to the flask. The residue was dissolved in MeOH and purified on HPLC (15% to 55% CH₃CN over 7.5 minutes with 0.1% TFA). Fractions containing the product were combined, concentrated and lyophilized to give 4-((28-((2,4-dinitrophenyl)amino)-13,16-dioxo-3,6,9,20,23,26-hexaoxa-12,17-diazaoctacosyl)oxy)-1H-indole-2-carboxylic acid (116 mg, 0.145 mmol, 78% yield) as a yellow oil/solid.

LCMS: m/z (M+H=793)

¹H NMR (400 MHz, METHANOL-d₄) δ ppm 9.00 (d, J=2.69 Hz, 1H) 8.24 (dd, J=9.54, 2.69 Hz, 1H) 7.10-7.23 (In, 3H) 7.03 (d, J=8.31 Hz, 1H) 6.51 (d, J=7.58 Hz, 1H) 4.22-4.32 (m, 2H) 3.96 (dd, J=5.26, 3.79 Hz, 2H) 3.76-3.84 (m, 4H) 3.56-3.75 (m, 18H) 3.49-3.54 (m, 4H) 2.46 (s, 4H)

### Step 6

### N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-4-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide trifluoroacetic acid salt

To a solution of 4-((28-((2,4-dinitrophenyl)amino)-13,16-dioxo-3,6,9,20,23,26-hexaoxa-12,17-diazaoctacosyl)oxy)-1H-indole-2-carboxylic acid (66.5 mg, 0.084 mmol)) in N,N-Dimethylformamide (1 mL) was added HATU (31.9 mg, 0.084 mmol) followed by DIPEA (0.066 mL, 0.378 mmol) and the resulting mixture was stirred at room temperature for 10 min. After this, a solution of (S)-2-((3S,3'S)-3′,6-dimethylspiro[indoline-3,4′-piperidin]-1′-yl)-1-(piperidin-4-yl)ethanol (28.8 mg, 0.084 mmol) in N,N-dimethylformamide (1 mL) was added and the mixture was stirred at room temperature for 20 minutes. The reaction mixture was directly purified on HPLC (15% to 50% CH₃CN over 7.5 minutes with 0.1% TFA). Fractions containing the product were combined, concentrated and lyophilized to give N1-(2-(2-(2-(2-((2-(4-((S)-2-((3S,3'S)-3′,6-dimethylspiro[indoline-3,4′-piperidin]-1′-yl)-1-hydroxyethyl)piperidine-1-carbonyl)-1H-indol-4-yl)oxy)ethoxy)ethoxy)ethoxy)ethyl)-N4-(2-(2-(2-(2-((2,4-dinitrophenyl)amino)ethoxy)ethoxy)ethoxy)ethyl)succinamide trifluoroacetic acid salt (60 mg, 0.048 mmol, 56.9% yield) as a yellow solid.

LCMS: m/z (M+H=1119)

¹H NMR (400 MHz, METHANOL-d₄) δ ppm 9.00 (d, J=2.69 Hz, 1H) 8.25 (dd, J=9.54, 2.69 Hz, 1H) 7.00-7.19 (m, 4H) 6.84-6.92 (m, 2H) 6.79 (s, 1H) 6.54 (d, J=7.58 Hz, 1H) 4.72 (br. s., 3H) 4.22-4.35 (m, 2H) 3.87-4.02 (m, 3H) 3.34-3.86 (m, 32H) 3.00-3.17 (m, 3H) 2.88 (t, J=12.59 Hz, 2H) 2.46 (s, 4H) 2.26-2.38 (m, 5H) 1.96-2.08 (m, 2H) 1.80 (d, J=10.76 Hz, 2H) 1.48 (br. s., 2H) 0.80 (d, J=6.85 Hz, 3H).

### (4-((S)-2-((3S,3'S)-3′,6-Dimethylspiro[indoline-3,4′-piperidin]-1′-yl)-1-hydroxyethyl)piperidin-1-yl)(6-(4-(4-(3-((2,4-dinitrophenyl)amino)propyl)piperazine-1-carbonyl)phenoxy)-1H-indol-2-yl)methanone

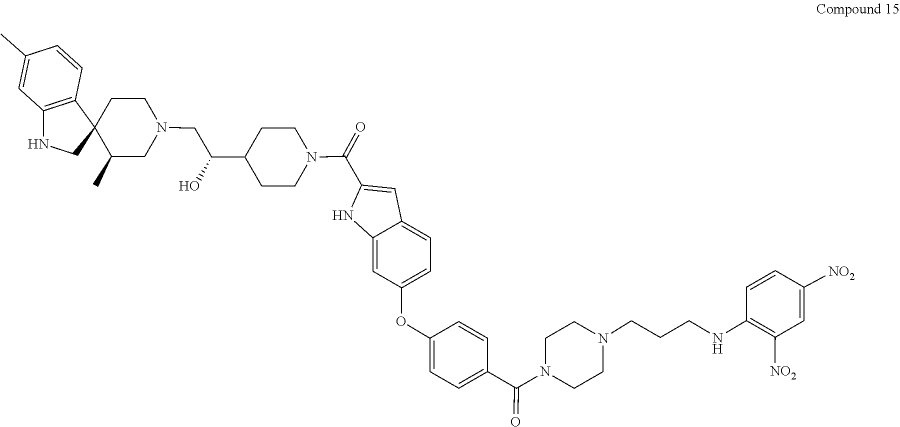 [SMILES: Cc1ccc2c(c1)NC[C@]21CCN(C[C@@H](O)C2CCN(C(=O)c3cc4ccc(Oc5ccc(C(=O)N6CCN(CCCNc7ccc([N+](=O)[O-])cc7[N+](=O)[O-])CC6)cc5)cc4[nH]3)CC2)C[C@H]1C]

### Step 1

### Tert-butyl 4-(3-((2,4-dinitrophenyl)amino)propyl)piperazine-1-carboxylate

To a stirred solution of tert-butyl 4-(3-aminopropyl)piperazine-1-carboxylate (2 g, 8.22 mmol), in dichloromethane (DCM) (30 mL) was added 1-fluoro-2,4-dinitrobenzene (1.529 g, 8.22 mmol) and Et₃N (2.291 mL, 16.44 mmol). The reaction mixture was stirred at 25° C. for 12 hr. Progress of the reaction was monitored by TLC (TLC system 50% EtOAc in Hexane, Rf:0.5). The reaction mixture was concentrated under reduced pressure to get crude compound as a pale yellow solid. The crude compound was triturated with diethyl ether and dried to afford tert-butyl 4-(3-((2,4-dinitrophenyl)amino)propyl)piperazine-1-carboxylate (2.7 g, 6.48 mmol, 79% yield) as a pale yellow solid. LCMS: m/z (M+H=410).

### Step 2

### 2,4-Dinitro-N-(3-(piperazin-1-yl)propyl)aniline, Hydrochloride

To a stirred solution of tert-butyl 4-(3-((2,4-dinitrophenyl)amino)propyl)piperazine-1-carboxylate, hydrochloride (1.2 g, 2.69 mmol), in 1,4-dioxane (15 mL) cooled to 0° C., was added HCl in Dioxane (5 mL, 20.00 mmol). The reaction mixture was stirred at 25° C. for 12 hr. Progress of the reaction was monitored by TLC (10% MeOH in DCM, Rf: 0.1, UV active). The reaction mixture was concentrated under reduced pressure to get crude compound as a brown gummy. The crude compound was triturated with ether and dried to afford compound 2,4-dinitro-N-(3-(piperazin-1-yl)propyl)aniline, hydrochloride (850 mg, 2.179 mmol, 81% yield) as a pale yellow solid. LCMS: m/z: (M+H=310), Rt: 1.54 min.

### Step 3

### Methyl 6-(4-(4-(3-((2,4-dinitrophenyl)amino)propyl)piperazine-1-carbonyl)phenoxy)-1H-indole-2-carboxylate

To a stirred solution of 4-((2-(methoxycarbonyl)-1H-indol-6-yl)oxy)benzoic acid (350 mg, 1.124 mmol) (Steps 1-2, Compound 12 above) in N,N-Dimethylformamide (DMF) (10 mL) at 0° C. was added DIPEA (0.589 mL, 3.37 mmol), HATU (641 mg, 1.687 mmol) and 2,4-dinitro-N-(3-(piperazin-1-yl)propyl)aniline, hydrochloride (389 mg, 1.124 mmol). The reaction mixture was stirred at 25° C. for 16 hr. Progress of the reaction was monitored by TLC (5% MeOH in DCM, Rf:0.4, UV active). The reaction mixture was diluted with ice cold water (30 mL), and stirred for 10 min. The precipitated solid was filtered and dried to afford methyl 6-(4-(4-(3-((2,4-dinitrophenyl)amino)propyl)piperazine-1-carbonyl)phenoxy)-1H-indole-2-carboxylate (350 mg, 0.518 mmol, 46.0% yield) as a yellow solid. LCMS: m/z (M+H=603), Rt: 1.68 min.

### Step 4

### 6-(4-(4-(3-((2,4-Dinitrophenyl)amino)propyl)piperazine-1-carbonyl)phenoxy)-1H-indole-2-carboxylic acid

To a stirred solution of methyl 6-(4-(4-(3-((2,4-dinitrophenyl)amino)propyl)piperazine-1-carbonyl)phenoxy)-1H-indole-2-carboxylate (250 mg, 0.415 mmol) in tetrahydrofuran (THF) (10 mL) was added 1N sodium hydroxide (10 mL, 10.00 mmol) at 0° C. The reaction mixture was stirred at 26° C. for 16 hr. Progress of the reaction was monitored by TLC. TLC shows starting material was consumed and a new, more polar, spot formed. The solvent (THF) was concentrated under reduced pressure, diluted with water and extracted with ethyl acetate. The aqueous layers were acidified with saturated citric acid solution and stirred for 10 min. The precipitated solid was filtered and dried under reduced pressure to give 6-(4-(4-(3-((2,4-dinitrophenyl)amino)propyl)piperazine-1-carbonyl)phenoxy)-1H-indole-2-carboxylic acid (180 mg, 0.293 mmol, 70.6% yield) as a pale yellow solid. LCMS: m/z: (M+H=589.07), Rt: 1.61 min.

### Step 5

### (4-((S)-2-((3S,3'S)-3′,6-Dimethylspiro[indoline-3,4′-piperidin]-1′-yl)-1-hydroxyethyl)piperidin-1-yl)(6-(4-(4-(3-((2,4-dinitrophenyl)amino)propyl)piperazine-1-carbonyl)phenoxy)-1H-indol-2-yl)methanone

To a stirred solution of 6-(4-(4-(3-((2,4-dinitrophenyl)amino)propyl)piperazine-1-carbonyl)phenoxy)-1H-indole-2-carboxylic acid (165 mg, 0.280 mmol) in N,N-Dimethylformamide (DMF) (6 mL) at 25° C. was added HATU (117 mg, 0.308 mmol), DIPEA (0.245 mL, 1.402 mmol) and (S)-2-((3S,3'S)-3′,6-dimethylspiro[indoline-3,4′-piperidin]-1′-yl)-1-(piperidin-4-yl)ethanol REFERENCE (96 mg,

0.280 mmol. The reaction mixture was stirred at 25° C. for 16 hr. Progress of the rection was monitored by TLC. (10% MeOH in DCM, Rf:0.3, UV active). The reaction mixture was diluted with cold water and stirred for 10 min. The precipitated solid was filtered and dried under reduced pressure to afford the crude compound as pale yellow solid. The crude compound was subjected to prep HPLC:

HPLC Method Conditions:

Column: X Bridge C18 (150×4.6 mm, 3.5 μm)

Mobile Phase: A: 0.1% TFA in Water, B: Acetonitrile

Gradient: % A/% B: 0/10,1/10,10/60,12/60,15/98,18/98,19/10,24/10

The acetonitrile prep solvent was reduced under reduced pressure, the resulting aqueous solution was basified with saturated sodium bicarbonate solution and extracted with dichlorometane. The organic layer was concentrated under reduced pressure and dried under lyophilization to give the title compound (4-((S)-2-((3S,3'S)-3′,6-dimethylspiro[indoline-3,4′-piperidin]-1′-yl)-1-hydroxyethyl)piperidin-1-yl)(6-(4-(4-(3-((2,4-dinitrophenyl)amino)propyl)piperazine-1-carbonyl)phenoxy)-1H-indol-2-yl)methanone as a pale yellow solid (19 mg, 0.018 mmol, 54% yield).

LCMS: m/z (M+H=914), Rt:4.47 min.

### Tertiary alcohol stereoisomers of (S)-3-((29-((2,4-dinitrophenyl)amino)-3,6,9,12,15,18,21,24,27-nonaoxanonacosyl)oxy)-N-(2-(3-((4-hydroxy-4-(5-(pyrimidin-2-yl)pyridin-2-yl)cyclohexyl)amino)pyrrolidin-1-yl)-2-oxoethyl)-5-(trifluoromethyl)benzamide

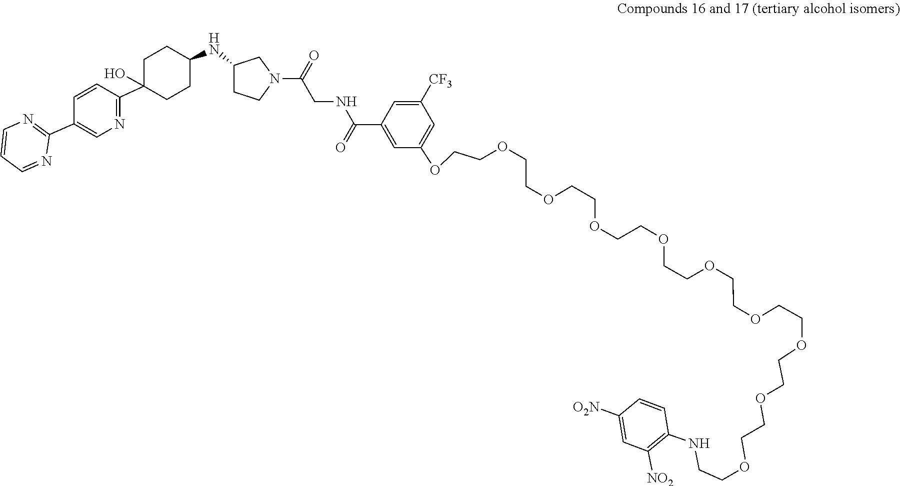

### Step 1

### Tert-butyl (29-hydroxy-3,6,9,12,15,18,21,24,27-nonaoxanonacosyl)carbamate

To a stirred solution of 29-amino-3,6,9,12,15,18,21,24,27-nonaoxanonacosan-1-ol (1 g, 2.186 mmol) in DCM (15 mL) was added Boc-anhydride (0.609 mL, 2.62 mmol) at rt under nitrogen atmosphere. The resulting reaction mixture was stirred at RT for 4 hr (TLC system: 10% methanol in ethyl acetate, Rf: 0.6, detection: UV inactive, iodine and ninhydrine charring active). Reaction mixture was extracted with DCM (3×20 mL). The combined organic layer was washed with brine solution (20 mL), dried over anhydrous Na₂SO₄, filtered and concentrated under reduced pressure to afford crude tert-butyl (29-hydroxy-3,6,9,12,15,18,21,24,27-nonaoxanonacosyl)carbamate (1.20 g, 2.149 mmol, 98% yield) as colourless liquid. LCMS: m/z (M+H=558), Rt: 2.6 min. The purity of the product was 99.86% by ELSD LCMS.

### Step 2

### 2,2-dimethyl-4-oxo-3,8,11,14,17,20,23,26,29,32-decaoxa-5-azatetratriacontan-34-yl 4-methylbenzenesulfonate

To a solution of tert-butyl (29-hydroxy-3,6,9,12,15,18,21,24,27-nonaoxanonacosyl)carbamate (600 mg, 1.076 mmol) in dichloromethane (DCM) (25 mL) was added TEA (0.900 mL, 6.46 mmol) and portion-wise p-toluene-sulfonyl-chloride (308 mg, 1.614 mmol) at −20° C. Then the reaction mixture was stirred at the same temperature for 16 h (TLC system: 5% MeOH in DCM; Rf 0.6, UV active). Reaction monitored by LCMS. After the completion of the reaction based on TLC and LCMS, the reaction mixture was quenched with cold water (20 mL) and extracted into DCM (3×50 ml). The combined organic layers were washed thoroughly with cold water (50 mL), the organic layer was dried over anhydrous Na₂SO₄, filtered and concentrated under reduced pressure.

The crude compound was purified by column chromatography, using neutral alumina and eluted with 50-60% ethyl acetate in pet ether. The collected pure fractions were distilled under reduced pressure to afford pure 2,2-dimethyl-4-oxo-3,8,11,14,17,20,23,26,29,32-decaoxa-5-azatetratriacontan-34-yl 4-methylbenzenesulfonate (680 mg, 0.881 mmol, 82% yield) as a colourless liquid. LCMS: m/z (M+H=712), Rt: 2.3 min.

### Step 3

### Methyl 3-hydroxy-5-(trifluoromethyl)benzoate

To a stirred solution of 3-hydroxy-5-(trifluoromethyl)benzoic acid (500 mg, 2.426 mmol) in methanol (10 mL) was added SOCl₂ (0.885 mL, 12.13 mmol) at rt under nitrogen atmosphere. The resulting reaction mixture was stirred at 65° C. for 4 hr. (TLC system: 50% ethyl acetate in pet ether, Rf: 0.7, Detection: UV). The reaction mixture was cooled to rt and the solvent (SOCl₂ and DCM) was removed under reduced pressure and the residue was quenched with cold water (20 mL) and basified with a saturated NaHCO₃ solution (10 mL) up to PH=8. The aqueous layer was extracted with DCM (3×50 mL), the combined organic layers were washed thoroughly with cold water (20 mL), dried over anhydrous Na₂SO₄, filtered and concentrated under reduced pressure to afford methyl 3-hydroxy-5-(trifluoromethyl)benzoate (500 mg, 2.259 mmol, 93% yield) as an off white solid. LCMS: m/z (M+H=219), Rt: 2.7 min.

### Step 4

### Methyl 3-((2,2-dimethyl-4-oxo-3,8,11,14,17,20,23,26,29,32-decaoxa-5-azatetratriacontan-34-yl)oxy)-5-(trifluoromethyl)benzoate

To a stirred suspension of 2,2-dimethyl-4-oxo-3,8,11,14,17,20,23,26,29,32-decaoxa-5-azatetratriacontan-34-yl 4-methylbenzenesulfonate (676 mg, 0.949 mmol) and methyl 3-hydroxy-5-(trifluoromethyl)benzoate (190 mg, 0.863 mmol) in acetonitrile (15 mL) was added K₂CO₃ (596 mg, 4.32 mmol) at 100° C. under nitrogen atmosphere. The resulting reaction mixture was heated at 100° C. for 16 hr. Progress of the reaction was monitored by TLC. (10% MeOH in ethylacetate; 0.4, UV active). The reaction mixture was partitioned between cold water (100 ml), extracted with EtOAc (3×50 ml), the organic extracts dried over anhydrous Na₂SO₄, filtered and concentrated under reduced pressure. The crude compound was purified by column chromatography using neutral alumina eluting with 100% ethyl acetate to afford methyl 3-((2,2-dimethyl-4-oxo-3,8,11,14,17,20,23,26,29,32-decaoxa-5-azatetratriacontan-34-yl)oxy)-5-(trifluoromethyl)benzoate (570 mg, 0.692 mmol, 80% yield) as a light yellow liquid. LCMS: m/z (M+H=760), Rt: 2.5 min.

### Step 5

### Methyl 3-((29-amino-3,6,9,12,15,18,21,24,27-nonaoxanonacosyl)oxy)-5-(trifluoromethyl)benzoate

To a stirred solution of methyl 3-((2,2-dimethyl-4-oxo-3,8,11,14,17,20,23,26,29,32-decaoxa-5-azatetratriacontan-34-yl)oxy)-5-(trifluoromethyl)benzoate (550 mg, 0.724 mmol) in dichloromethane (DCM) (20 mL) was added TFA (0.01521 mL, 0.197 mmol) at 0° C. The resulting reaction mixture was stirred at rt for 4 hr (TLC system: 100% ethyl acetate, Rf: 0.1, Detection: UV). The reaction mixture was concentrated (TFA and DCM) under reduced pressure, the residue quenched with cold water (50 mL), and basified with saturated NaHCO₃ solution (50 mL, 10%) up to PH=8. The aqueous layer was extracted into DCM (3×50 mL), the combined organic layer was washed thoroughly with cold water (100 mL), dried over anhydrous Na₂SO₄, filtered and concentrated under reduced pressure to afford methyl 3-((29-amino-3,6,9,12,15,18,21,24,27-nonaoxanonacosyl)oxy)-5-(trifluoromethyl)benzoate (490 mg, 0.718 mmol, 99% yield) as a colourless liquid. LCMS: m/z (M+H=660), Rt: 1.9 min. The purity of the compound was 96%.

### Step 6

### Methyl 3-((29-((2,4-dinitrophenyl)amino)-3,6,9,12,15,18,21,24,27-nonaoxanonacosyl)oxy)-5-(trifluoromethyl)benzoate

To a solution of methyl 3-((29-amino-3,6,9,12,15,18,21,24,27-nonaoxanonacosyl)oxy)-5-(trifluoromethyl)benzoate (500 mg, 0.758 mmol) in acetonitrile (50 mL) were added Cs₂CO₃ (494 mg, 1.516 mmol) and the reaction was stirred at rt for 10 min. Then to the above reaction was added 1-chloro-2,4-dinitrobenzene (154 mg, 0.758 mmol) and the mixture was stirred at rt for 16 h (TLC system: 5% MeOH in DCM; Rf 0.4, UV active). Progress of the reaction was monitored by TLC and LCMS. After the completion of the reaction by TLC and LCMS, the reaction mass was quenched with ice cold water and extracted into ethyl acetate (3×100 mL). The combined organics were washed thoroughly with cold water (100 ml), the organic layer was dried over anhydrous Na₂SO₄, filtered and concentrated under reduced pressure. The crude compound was purified by column chromatography using neutral alumina and was eluted with 50-60% ethyl acetate in pet ether. The collected pure fractions were distilled under reduced pressure to afford pure methyl 3-((29-((2,4-dinitrophenyl)amino)-3,6,9,12,15,18,21,24,27-nonaoxanonacosyl)oxy)-5-(trifluoromethyl)benzoate (460 mg, 0.485 mmol, 64.0% yield) as a yellow colored semi solid. LCMS: m/z (M−H=824), Rt: 2.2 min. The purity of the compound was 87%.

### Step 7

### 3-((29-((2,4-Dinitrophenyl)amino)-3,6,9,12,15,18,21,24,27-nonaoxanonacosyl)oxy)-5-(trifluoromethyl)benzoic acid

To a stirred solution of methyl 3-((29-((2,4-dinitrophenyl)amino)-3,6,9,12,15,18,21,24,27-nonaoxanonacosyl)oxy)-5-(trifluoromethyl)benzoate (450 mg, 0.545 mmol) in tetrahydrofuran (THF) (50 mL) and water (10 mL) was added LiOH.H₂O (39.2 mg, 1.635 mmol) at 0° C. temperature. The reaction was stirred at rt for 4 hr. Progress of the reaction was monitored by TLC and LCMS. (100% ethyl acetate, rf value: 0.4, UV active). After the completion of the reaction by TLC and LCMS, the reaction mass was concentrated under reduced pressure. The resulting residue was diluted with cold water (50 ml), acidified with saturated aqueous NH₄Cl, (20 mL), extracted with DCM (3×50 mL), the combined organic layers were dried over anhydrous Na₂SO₄, filtered and concentrated under reduced pressure to afford the crude compound. The crude compound was partially dissolved with pentane, triturated, and filtered to afford pure 3-((29-((2,4-dinitrophenyl)amino)-3,6,9,12,15,18,21,24,27-nonaoxanonacosyl)oxy)-5-(trifluoromethyl)benzoic acid (400 mg, 0.457 mmol, 84% yield) as a yellow semi solid. LCMS: m/z (M−H=812), Rt: 1.8 min. The purity of the desired product by LCMS was 93%.

### Step 8

### 1-(2-Benzyloxycarbonylamino-acetyl)-pyrrolidin-3-yl]-carbamic acid tert-butyl ester

To a stirring solution of 2-(((benzyloxy)carbonyl)amino)acetic acid (5 g, 23.90 mmol) in pyridine (50 mL) under nitrogen at 0° C. was added EDC.HCl (13.75 g, 71.7 mmol). The reaction mixture was stirred at rt for 30 min, then to the above reaction mixture was added (S)-tert-butyl pyrrolidin-3-ylcarbamate (5.34 g, 28.7 mmol) and stirred at rt for 16 h.

Progress of the reaction was monitored by TLC (100% EtOAc, Rf: 0.5). The reaction was then concentrated under reduced pressure to remove all the pyridine and the residue obtained was diluted with ice cold water (150 mL). The resulting solid obtained was washed thoroughly with cold water (150 mL), filtered, and dried under vaccum to afford pure product 1-(2-benzyloxycarbonylamino-acetyl)-pyrrolidin-3-yl]-carbamic acid tert-butyl ester (5.5 g, 8.35 mmol, 34.9% yield). LCMS: m/z (M+H=378), Rt: 1.7 min.

### Step 9

### (S)-benzyl (2-(3-aminopyrrolidin-1-yl)-2-oxoethyl)carbamate, Hydrochloride

To a stirring solution of [1-(2-benzyloxycarbonylamino-acetyl)-pyrrolidin-3-yl]-carbamic acid tert-butyl ester (5.5 g, 14.57 mmol) in 1,4-dioxane (50 mL) under nitrogen at 0° C. was added HCl (50 mL, 200 mmol). The reaction mixture was stirred at rt for 2 hrs. Progress of the reaction was monitored by TLC (100% EtOAc, Rf: 0.2). After the completion of the reaction by TLC and LCMS, the reaction solvent was evaporated under reduced pressure. The crude material was partially dissolved/triturated with diethyl ether (3×40 ml), and finally dried under reduced pressure to obtain the desired product (S)-benzyl (2-(3-aminopyrrolidin-1-yl)-2-oxoethyl)carbamate, hydrochloride (4.0 g, 11.38 mmol, 78% yield) as a yellow solid. LCMS: m/z (M+H=278), Rt: 1.1 min. The purity was determined to be 89% by LCMS.

### Step 10

### Isomer-1 and Isomer-2 of (S)-benzyl (2-(3-((4-hydroxy-4-(5-(pyrimidin-2-yl)pyridin-2-yl)cyclohexyl)amino)pyrrolidin-1-yl)-2-oxoethyl)carbamate

To a stirred solution of 4-hydroxy-4-(5-(pyrimidin-2-yl)pyridin-2-yl)cyclohexanone (300 mg, 1.114 mmol) (Metcalf, B. et al. ACS Med. Chem. Lett. 2005) in 2-butanol (50 mL) was added (S)-benzyl (2-(3-aminopyrrolidin-1-yl)-2-oxoethyl)carbamate (309 mg, 1.114 mmol) and triethylamine (0.466 mL, 3.34 mmol) at 25° C. The mixture was stirred for 30 min at RT. To this mixture was added sodium triacetoxyborohydride (472 mg, 2.228 mmol) at 0° C. 4 h. The reaction was monitored by TLC (10% MeOH in DCM, Rf: 0.4, UV active). After completion of the reaction, the residue was partitioned between water and ethyl acetate (100 mL). The organic layer was separated, dried over anhydrous sodium sulphate, filtered, and the filtrate was dried under vacuum to afford the crude product. The crude product was purified by reverse phase Grace column chromatography (C18, 40 g), employing 70% acetonitrile and water as the eluents, to afford a mixture (430 mg, LC-MS purity 97%) of geometrical isomers. The isomers were then separated using PREP HPLC to afford two discrete isomers. Isomer-1 (S)-benzyl (2-(3-((4-hydroxy-4-(5-(pyrimidin-2-yl)pyridin-2-yl)cyclohexyl)amino)pyrrolidin-1-yl)-2-oxoethyl)carbamate (68 mg, 0.116 mmol, 20.8% yield) was isolated as an off white solid; LCMS: m/z (M+H=531). Isomer-2 (S)-benzyl (2-(3-((4-hydroxy-4-(5-(pyrimidin-2-yl)pyridin-2-yl)cyclohexyl)amino)pyrrolidin-1-yl)-2-oxoethyl)carbamate (64 mg, 0.115 mmol, 20.69% yield) was isolated as an off white solid. LCMS: m/z (M−H=529). PREP HPLC conditions: Mobile phase-A: Trifluoroacetic acid. Mobile phase-B: Acetonitrile. COLUMN: PURITAS C18 (250*30 mm dimensions, 10 μm particle size). METHOD: (A:B) (75:25) Isocratic. FLOW: 30 ml/min.

Isomer-1 and Isomer-2 were further characterized using analytical, normal phase chromatography. Column: Chiralcel-OX—H (250×4.6 mm, 5 μm)

Mobile Phase A:0.2% DEA in n-Hexane

Mobile phase B:0.2% DEA in Ethanol

Isocratic (A:B):35:65

Flow rate:1.0 mL/min

Isomer-1 eluted at 24.4 minutes (fast eluting)

Isomer-2 eluted at 29.9 minutes (slow eluting)

### Step 11

### (S)-2-amino-1-(3-((4-hydroxy-4-(5-(pyrimidin-2-yl)pyridin-2-yl)cyclohexyl)amino)pyrrolidin-1-yl)ethanone derived from Isomer-1 in Step 10

To a stirred solution of Isomer-1 (S)-benzyl (2-(3-((4-hydroxy-4-(5-(pyrimidin-2-yl)pyridin-2-yl)cyclohexyl)amino)pyrrolidin-1-yl)-2-oxoethyl)carbamate (50 mg, 0.094 mmol) in methanol (10 mL) was added 10% Pd/C (20.06 mg, 0.019 mmol) at 25° C. The reaction was stirred for 4 hr under H₂ gas balloon at room temperature. After completion of the reaction, the reaction mass was filtered through a celite bed using a buchner funnel, and the filtrate was dried under vacuum. The filtrate was then washed with 10% diethyl ether in petroleum ether to afford (S)-2-amino-1-(3-((4-hydroxy-4-(5-(pyrimidin-2-yl)pyridin-2-yl)cyclohexyl)amino)pyrrolidin-1-yl)ethanone (30 mg, 0.044 mmol, 46.6% yield) as off white solid. LCMS: m/z (M+H=397).

### Step 12

### Compound 16

### (S)-3-((29-((2,4-dinitrophenyl)amino)-3,6,9,12,15,18,21,24,27-nonaoxanonacosyl)oxy)-N-(2-(3-((4-hydroxy-4-(5-(pyrimidin-2-yl)pyridin-2-yl)cyclohexyl)amino)pyrrolidin-1-yl)-2-oxoethyl)-5-(trifluoromethyl)benzamide derived from Isomer-1 in Step 10

To a stirred solution of 3-((29-((2,4-dinitrophenyl)amino)-3,6,9,12,15,18,21,24,27-nonaoxanonacosyl)oxy)-5-(trifluoromethyl)benzoic acid (50 mg, 0.062 mmol) in pyridine (10 mL) was added EDC (23.62 mg, 0.123 mmol) at rt. The reaction mixture was stirred for 30 min and then (S)-2-amino-1-(3-((4-hydroxy-4-(5-(pyrimidin-2-yl)pyridin-2-yl)cyclohexyl)amino)pyrrolidin-1-yl)ethanone (24.42 mg, 0.062 mmol) was added at rt. The resulting mixture was stirred at rt for 16h. The reaction progress was monitored by TLC until starting material was consumed (TLC mobile phase: 10% meOH in EtOAc: Rf: 0.4, UV active). The reaction mixture was poured into ice water (50 mL) and extracted with ethyl acetate (3×50 mL), dried over anhydrous Na₂SO₄, filtered and concentrated under reduced pressure to afford the crude product. The crude product was purified by prep HPLC.

Prep HPLC conditions:

Mobile Phase A:10 mM Ammonium bicarbonate (Aq) Mobile Phase B: Acetonitrile. Column: Xselect CSH phenyl hexyl 150*19 mm, 5 um. Method % A/% B=0/30, 1/30, 10/65, 11/65, 11.5/100.

Flow: 18 ml/min. Temp: Ambient.

The pure fractions were distilled under reduced pressure and the obtained residue was lyophilised to afford pure (S)-3-((29-((2,4-dinitrophenyl)amino)-3,6,9,12,15,18,21,24,27-nonaoxanonacosyl)oxy)-N-(2-(3-((4-hydroxy-4-(5-(pyrimidin-2-yl)pyridin-2-yl)cyclohexyl)amino)pyrrolidin-1-yl)-2-oxoethyl)-5-(trifluoromethyl)benzamide (20 mg, 0.016 mmol, 26.7% yield) as a pale yellow semi solid. LCMS: m/z (M+H=1190), Rt: 2.4 min.

### Step 13

### (S)-2-amino-1-(3-((4-hydroxy-4-(5-(pyrimidin-2-yl)pyridin-2-yl)cyclohexyl)amino)pyrrolidin-1-yl)ethanone derived from Isomer-2 in step 10

To a stirred solution of Isomer-2 (S)-benzyl (2-(3-((4-hydroxy-4-(5-(pyrimidin-2-yl)pyridin-2-yl)cyclohexyl)amino)pyrrolidin-1-yl)-2-oxoethyl)carbamate (60 mg, 0.113 mmol) in methanol (10 mL) was added 10% Pd/C (24.07 mg, 0.023 mmol) at 25° C. The reaction mixture was stirred for 4 hr at RT under hydrogen gas using a balloon. After completion of the reaction (TLC: 10% MeOH in DCM, starting material was shown to be consumed), the reaction mix was filtered through a celite bed using a buchner funnel. The filtrate was concenrtrated under reduced pressure to afford crude product. The crude compound was washed with 10% diethyl ether in pet-ether to afford (S)-2-amino-1-(3-((4-hydroxy-4-(5-(pyrimidin-2-yl)pyridin-2-yl)cyclohexyl)amino)pyrrolidin-1-yl)ethanone (40 mg, 0.069 mmol, 60.7% yield) as an off white solid. LCMS: m/z (M−H=397), Rt: 1.2 min.

### Step 14

### Compound 17

### (S)-3-((29-((2,4-dinitrophenyl)amino)-3,6,9,12,15,18,21,24,27-nonaoxanonacosyl)oxy)-N-(2-(3-((4-hydroxy-4-(5-(pyrimidin-2-yl)pyridin-2-yl)cyclohexyl)amino)pyrrolidin-1-yl)-2-oxoethyl)-5-(trifluoromethyl)benzamide derived from Isomer-2 in Step 10

To a stirred solution of 3-((29-((2,4-dinitrophenyl)amino)-3,6,9,12,15,18,21,24,27-nonaoxanonacosyl)oxy)-5-(trifluoromethyl)benzoic acid (50 mg, 0.062 mmol) in pyridine (3 mL) was added EDC.HCl (23.62 mg, 0.123 mmol) at RT. The reaction mixture was stirred for 30 min and then (S)-2-amino-1-(3-((4-hydroxy-4-(5-(pyrimidin-2-yl)pyridin-2-yl)cyclohexyl)amino)pyrrolidin-1-yl)ethanone (24.42 mg, 0.062 mmol) was added at rt. The resulting mixture was stirred at rt for 16 hr. The reaction progress was monitored by TLC until starting material was consumed (TLC: 100% EtOAc: Rf-0.3, UV active). The reaction mixture was poured into ice water (50 mL) and extracted with ethyl acetate (3×50 mL), dried over anhydrous Na₂SO₄, filtered, and the filtrate was concentrated under reduced pressure. The crude compound was purified by silica gel column chromatography. The compound was further purified by PREP HPLC to afford (S)-3-((29-((2,4-dinitrophenyl)amino)-3,6,9,12,15,18,21,24,27-nonaoxanonacosyl)oxy)-N-(2-(3-((4-hydroxy-4-(5-(pyrimidin-2-yl)pyridin-2-yl)cyclohexyl)amino)pyrrolidin-1-yl)-2-oxoethyl)-5-(trifluoromethyl)benzamide (15 mg, 0.012 mmol, 20.22% yield) as a single stereoisomer, and as a yellow coloured gummy solid. LCMS: m/z (M+H=1190), Rt: 2.4 min.

PREP HPLC conditions:

MOBILE PHASE A: 0.1% TFA (water), MOBILE PHASE B: Acetonitrile, COLUMN:—XBridge C18 150*19 mm, 5 um. FLOW: 16 ml/min, METHOD: (% A/% B):—0/10, 10/45, 13.8/45,14/100, 17/100, 17.2/10, 20/10. TEMPERATURE: Ambient.

### 2-(4-(4-(Tert-butyl)piperazine-2-carbonyl)-1-((5-chloro-4-(trifluoromethyl)thiazol-2-yl)carbam oyl)piperazin-2-yl)ethyl (29-((2,4-dinitrophenyl)amino)-3,6,9,12,15,18,21,24,27-nonaoxanonacosyl)carbamate, Trifluoroacetic acid salt

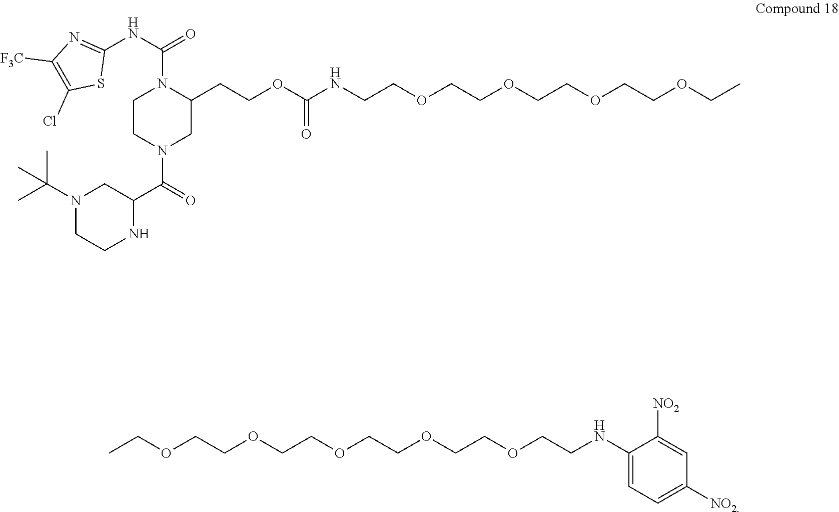

### Step 1

### Phenyl (5-chloro-4-(trifluoromethyl)thiazol-2-yl)carbamate

To an ice-cooled solution of 5-chloro-4-(trifluoromethyl)thiazol-2-amine (7.38 g, 32.4 mmol) and pyridine (5.25 mL, 64.9 mmol) in dichloromethane (DCM) (65 mL) was added phenyl carbonochloridate (4.27 mL, 34.1 mmol) dropwise. The resulting mixture was stirred at 0° C. for 1h, quenched with 0.3N HCl (60 mL) and then stirred for 10 min. The organic layer was separated, dried over MgSO₄, concentrated under reduced pressure and stored in a freezer. The residue solidified to a wax-like yellow solid which was dried under vacuum to afford the product, phenyl (5-chloro-4-(trifluoromethyl)thiazol-2-yl)carbamate. LC/MS: m/z 323.1 (M+H)⁺, 1.27 min (ret. time). ¹H NMR (400 MHz, DMSO-d₆) δ 7.17-7.59 (m, 5H); ¹⁹F NMR (376 MHz, DMSO-d₆) δ−61.04.

### Step 2

### (±) tert-butyl 4-((5-chloro-4-(trifluoromethyl)thiazol-2-yl)carbamoyl)-3-(2-hydroxyethyl)piperazine-1-carboxylate

A mixture of phenyl (5-chloro-4-(trifluoromethyl)thiazol-2-yl)carbamate (1.75 g, 4.34 mmol), (±) tert-butyl 3-(2-hydroxyethyl)piperazine-1-carboxylate (0.6 g, 2.61 mmol) and TEA (0.908 mL, 6.51 mmol) in ethanol (5 mL) was heated via microwave at 80° C. for 3 min. The mixture was then cooled and concentrated in vacuo. The residue was purified by normal phase chromatography (40 g ISCO gold silica gel column, 40 mL/min flow rate, gradient 10-50% EtOAc/hexanes for 15 column volumes and 50% EtOAc/hexanes for 5 column volumes) to afford (±) tert-butyl 4-((5-chloro-4-(trifluoromethyl)thiazol-2-yl)carbamoyl)-3-(2-hydroxyethyl)piperazine-1-carboxylate (1.10 g, 2.157 mmol, 83% yield) as a pale yellow solid. LC/MS: m/z 459.2 (M+H)⁺, 1.16 min (ret. time). 1H NMR (400 MHz, DMSO-d6) δ 11.56 (br. s, 1H), 4.71 (br. s, 1H), 4.27-4.47 (m, 1H), 3.69-3.99 (m, 3H), 3.43 (m, 2H), 2.71-3.13 (m, 3H), 1.67 (m, 2H), 1.42 (s, 9H)

### Step 3

### (±) Tert-butyl 4-(tert-butyl)-2-(4-((5-chloro-4-(trifluoromethyl)thiazol-2-yl)carbamoyl)-3-(2-hydroxyethyl)piperazine-1-carbonyl)piperazine-1-carboxylate

A solution of (±)tert-butyl 4-((5-chloro-4-(trifluoromethyl)thiazol-2-yl)carbamoyl)-3-(2-hydroxyethyl)piperazine-1-carboxylate (1.1 g, 2.397 mmol) in DCM (10 mL) was treated with 4M HCl/dioxane (5 mL, 20.00 mmol) at rt for 2.5 h or until the starting material was consumed. All volatiles were removed under reduced pressure. The residue was suspended in DCM (20 mL) and treated with N,N-diisopropylethylamine (1.465 mL, 8.39 mmol) to release the free base. (±)1-(Tert-butoxycarbonyl)-4-(tert-butyl)piperazine-2-carboxylic acid (0.755 g, 2.64 mmol) (prepared according to John Cumming, Alan Wellington Faull and David Waterson, US patent 2010/0152197) and HATU (1.367 g, 3.60 mmol) were added to the reaction mixture. The mixture was stirred at rt overnight and then concentrated in vacuo. The oil residue was purified by normal phase chromatography (40 g ISCO gold silica gel column, 40 mL/min flow rate, EtOAc with 1% TEA for 12 column volumes and 30% EtOH/EtOAc for 8 column volumes) to afford (±) tert-butyl 4-(tert-butyl)-2-(4-((5-chloro-4-(trifluoromethyl)thiazol-2-yl)carbamoyl)-3-(2-hydroxyethyl)piperazine-1-carbonyl)piperazine-1-carboxylate (0.377 g, 0.601 mmol, 25.08% yield) as an off-white film.

LC/MS: m/z 627.3 (M+H)⁺, 0.77 min (ret. time).

¹H NMR (400 MHz, CHLOROFORM-d) δ 4.70 (br. s, 1H), 4.10-4.45 (m, 3H), 3.41-3.82 (m, 6H), 3.18-3.35 (m, 1H), 2.78-3.11 (m, 4H), 2.32-2.55 (m, 1H), 2.07-2.22 (m, 1H), 1.71-1.94 (m, 2H), 1.36 (s, 9H), 0.94 (s, 9H).

### Step 4

### (±)Tert-butyl 4-(tert-butyl)-2-(4-((5-chloro-4-(trifluoromethyl)thiazol-2-yl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate

Into a solution of tert-butyl 4-(tert-butyl)-2-(4-((5-chloro-4-(trifluoromethyl)thiazol-2-yl)carbamoyl)-3-(2-hydroxyethyl)piperazine-1-carbonyl)piperazine-1-carboxylate (50 mg, 0.080 mmol) and TEA (0.022 mL, 0.159 mmol) in DCM (2 mL) was added phenyl carbonochloridate (10.00 μl, 0.080 mmol) slowly. The mixture was stirred at rt overnight. LCMS revealed a complete conversion to product. The solvent was removed in vacuo. The oil residue was purfied by normal phase chromatography (12 g ISCO gold silica gel column, 30 mL/min flow rate of 50-100% EtOAc in hexanes for 7 column volumes and straight EtOAc for 10 column volumes) to yield tert-butyl 4-(tert-butyl)-2-(4-((5-chloro-4-(trifluoromethyl)thiazol-2-yl)carbamoyl)-3-(2-((phenoxycarbonyl)oxy)ethyl)piperazine-1-carbonyl)piperazine-1-carboxylate (48.8 mg, LCMS: m/z 747.1 (M+H)⁺).

The mixture of the carbonate obtained above (26 mg), TEA (0.022 mL, 0.159 mmol) and N¹-(2,4-dinitrophenyl)-3,6,9,12,15,18,21,24,27-nonaoxanonacosane-1,29-diamine (27 mg, 0.043 mmol) in EtOH (1 mL) was heated at 40-50° C. over a weekend (˜65 h). LCMS revealed the starting carbonate was consumed and the desired product was formed based on a peak at m/z=638.3 (M+2H)²⁺. The reaction mixture was concentrated and purified by normal phase chromatography (12 g ISCO gold silica gel column, 30 mL/min flow rate of 50-100% EtOAc in hexanes for 5CVs, straight EtOAc for 5 column volumes and 30% EtOH/EtOAc for 20 column volumes). The desired product was eluted during column volume 12-18th. Concentrating the fractions afforded (±)tert-butyl 4-(tert-butyl)-2-(4-((5-chloro-4-(trifluoromethyl)thiazol-2-yl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate as a yellow film (16.9 mg, 38% yield). LC/MS: m/z 638.3 (M+2H)²+, 1.00 min (ret. time).

¹H NMR (400 MHz, CHLOROFORM-d) δ 9.57-10.16 (m, 1H), 9.16 (d, J=2.69 Hz, 1H), 8.82 (br. s, 1H), 8.29 (dd, J=2.32, 9.17 Hz, 1H), 6.98 (d, J=9.54 Hz, 1H), 6.41-6.80 (m, 1H), 4.76 (br. s, 1H), 4.38-4.64 (m, 1H), 4.04-4.32 (m, 2H), 3.81-3.93 (m, 3H), 3.50-3.79 (m, 41H), 2.77-3.49 (m, 7H), 2.41-2.62 (m, 1H), 1.84-2.32 (m, 2H), 1.44 (s, 9H), 1.04 (s, 9H).

### Step 5

### (±)2-(4-(4-(Tert-butyl)piperazine-2-carbonyl)-1-((5-chloro-4-(trifluoromethyl)thiazol-2-yl)carbam oyl)piperazin-2-yl)ethyl (29-((2,4-dinitrophenyl)amino)-3,6,9,12,15,18,21,24,27-nonaoxanonacosyl)carbamate, Trifluoroacetic acid salt

Into the solution of tert-butyl 4-(tert-butyl)-2-(4-((5-chloro-4-(trifluoromethyl)thiazol-2-yl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate (16.9 mg, 0.013 mmol) in dichloromethane (DCM) (0.5 mL) was added TFA (0.5 mL, 6.49 mmol) slowly. The mixture was stirred at rt for 1h and stored in a fridge overnight. LCMS revealed a complete conversion. The volatiles were removed in vacuo. The oil residue was dissolved in DMSO (1 mL) and purified by preparative HPLC (Gilson Luna acidic: Agilent Eclipse plus C18, 5 μm, 30×50 mm, 30-60% gradient, Acetonitrile/water with 0.1% TFA, 47 mL/min flow rate, 14 min run time, one injection) to afford (±)2-(4-(4-(tert-butyl)piperazine-2-carbonyl)-1-((5-chloro-4-(trifluoromethyl)thiazol-2-yl)carbam oyl)piperazin-2-yl)ethyl (29-((2,4-dinitrophenyl)amino)-3,6,9,12,15,18,21,24,27-nonaoxanonacosyl)carbamate, trifluoroacetic acid salt. It was isolated as a mixture of stereoisomers (11.8 mg, 67.7% yield) and as a yellow film. LC/MS (ESI): m/z 1175.0 (M+H)⁺, 0.89 min (ret. time), 98% purity.

¹H NMR (400 MHz, METHANOL-d₄) δ 9.05 (d, J=2.69 Hz, 1H), 8.31 (dd, J=2.69, 9.6 Hz 1H), 7.25 (d, J=9.6 Hz, 1H), 4.35-4.67 (m, 2H), 3.98-4.24 (m, 4H), 3.83 (t, J=5.38 Hz, 2H), 3.55-3.77 (m, 38H), 3.37-3.54 (m, 4H), 2.94-3.29 (m, 6H), 1.83-2.10 (m, 2H), 1.40-1.50 (m, 9H).

### 2-(4-(4-(Tert-butyl)piperazine-2-carbonyl)-1-((4-chloro-3-fluorophenyl)carbamoyl)piperazin-2-yl)ethyl (29-((2,4-dinitrophenyl)amino)-3,6,9,12,15,18,21,24,27-nonaoxanonacosyl)carbamate

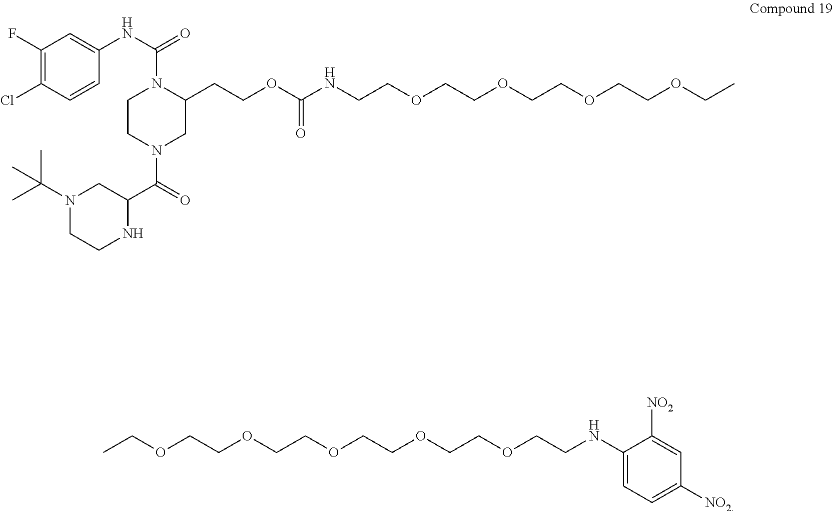

### Step 1

### Phenyl (4-chloro-3-fluorophenyl)carbamate

At 0° C., to a mixture of 4-chloro-3-fluoroaniline (7 g, 48.1 mmol) in DCM (100 mL) was added pyridine (7.8 mL, 96 mmol), followed by phenyl carbonochloridate (6.3 mL, 50.5 mmol). The mixture was stirred at 0° C. for 1 h, then warmed up to rt overnight. The mixture was washed with 1N HCl and back extracted with DCM. The combined organics were washed with brine, dried over anhydrous Na₂SO₄, and filtered. The filtrate was concentrated to give phenyl (4-chloro-3-fluorophenyl)carbamate (12 g, 45.2 mmol, 94% yield) as a colorless solid. The crude product was used without further purification.

LC/MS (ESI): m/z 266.1 (M+H)⁺, 1.13 min (ret. time).

### Step 2

### N-(4-Chloro-3-fluorophenyl)-2-(2-hydroxyethyl)piperazine-1-carboxamide

Phenyl (4-chloro-3-fluorophenyl)carbamate (441 mg, 1.659 mmol) and tert-butyl 3-(2-hydroxyethyl)piperazine-1-carboxylate (382 mg, 1.659 mmol) were suspended in EtOH (5 mL) and heated in a Biotage Initiator using initial high setting to 100° C. for 7 min. The reaction was concentrated under a stream of nitrogen at 50° C. then dissolved in DCM (3 mL) and TFA (3 mL, 38.9 mmol). After 30 minutes the reaction was concentrated under a stream of nitrogen at 50° C. The residue was dissolved in DCM/MeOH and loaded onto a SCX-3 cartridge (2 g). The cartridge was washed with 3 volumes of MeOH, then eluted with 3 volumes of 2N NH₃/MeOH. The eluants were concentrated under a stream of nitrogen at 50° C. followed by high vacuum, resulting in the isolation of N-(4-chloro-3-fluorophenyl)-2-(2-hydroxyethyl)piperazine-1-carboxamide (494 mg, 1.637 mmol, 99% yield) as a white solid.

LC/MS (ESI): m/z 302.0 (M+H)⁺, 0.39 min (ret. time).

### Step 3

### Tert-butyl 4-(tert-butyl)-2-(4-((4-chloro-3-fluorophenyl)carbamoyl)-3-(2-hydroxyethyl)piperazine-1-carbonyl)piperazine-1-carboxylate

N-(4-chloro-3-fluorophenyl)-2-(2-hydroxyethyl)piperazine-1-carboxamide (494 mg, 1.637 mmol) and 1-(tert-butoxycarbonyl)-4-(tert-butyl)piperazine-2-carboxylic acid (516 mg, 1.801 mmol) were dissolved in DMF (16 mL). HATU (747 mg, 1.965 mmol) was added, followed by DIPEA (1.4 mL, 8.2 mmol). The reaction was heated at 40° C. (external) for 1 h. The reaction was then diluted with 10% LiCl (50 mL), and extracted with EtOAc (16 mL) three times. The combined organic layers were concentrated onto Biotage isolute (under a stream of nitrogen at 50° C.). The crude product/isolute was purified on a silica cartridge (24 g) with a Combiflash Rf 200i, eluting at 35 mL/min using a non-linear 0-15% 10% NH₄OH in MeOH/DCM gradient. The desired fractions were concentrated under reduced pressure and dried under high vacuum, giving tert-butyl 4-(tert-butyl)-2-(4-((4-chloro-3-fluorophenyl)carbamoyl)-3-(2-hydroxyethyl)piperazine-1-carbonyl)piperazine-1-carboxylate (865 mg, 1.426 mmol, 87% yield) as a white solid. The isolated compound was assumed to be a mixture of diastereomers, but they were not distinguishable by LCMS/HPLC/NMR at this stage.

LC/MS (ESI): m/z 570.3 (M+H)⁺, 0.69 min (ret. time)

Anal. HPLC: 11.314 min (ret. time); Column: Luna C18 (2) 4.6×150 mm, 3 um. Method: 2-95% gradient (0.1% TFA in ACN/water) over 18 min, then held at 95% water 5% (0.1% TFA in ACN) for an additional 2 min.

### Step 4

### Isomers 1 and 2 of tert-butyl 4-(tert-butyl)-2-(4-((4-chloro-3-fluorophenyl)carbamoyl)-3-(2-((phenoxycarbonyl)oxy)ethyl)piperazine-1-carbonyl)piperazine-1-carboxylate

Tert-butyl 4-(tert-butyl)-2-(4-((4-chloro-3-fluorophenyl)carbamoyl)-3-(2-hydroxyethyl)piperazine-1-carbonyl)piperazine-1-carboxylate (180 mg, 0.316 mmol) was dissolved in DCM (2.1 mL). TEA (0.13 mL, 0.93 mmol) was added, followed by phenyl carbonochloridate (0.06 mL, 0.48 mmol). The reaction was stirred for 40 min, then washed with water and the organic layer was concentrated onto isolute. The crude product/isolute was purified on a silica cartridge (12 g) with a Combiflash Rf 200i, eluting at 30 mL/min using a non-linear 0-70% (2% NH₄OH in 1:3 EtOH/EtOAc)/hexanes gradient. The desired fractions were concentrated under reduced pressure and dried under high vacuum. The initial fractions were collected leading to the isolation of the shorter retention time racemic diastereomer, isomer 1, tert-butyl 4-(tert-butyl)-2-(4-((4-chloro-3-fluorophenyl)carbamoyl)-3-(2-((phenoxycarbonyl)oxy)ethyl)piperazine-1-carbonyl)piperazine-1-carboxylate (35 mg, 0.051 mmol, 16.06% yield). Note: Racemic Isomer 1 was isolated in pure form as a clear film. The subsequent fractions isolated gave rise to a mixture of racemic Isomers 1 and 2 (140 mg). This mixture of racemic diastereomers (140 mg) was further purified on a silica cartridge (24 g) with a Combiflash Rf 200i, eluting at 35 mL/min with an isocratic elution at 20% (2% NH₄OH in 1:3 EtOH/EtOAc)/hexanes. The desired fractions were concentrated under reduced pressure and dried under high vacuum leading to the isolation of Isomer 2 of tert-butyl 4-(tert-butyl)-2-(4-((4-chloro-3-fluorophenyl)carbamoyl)-3-(2-((phenoxycarbonyl)oxy)ethyl)piperazine-1-carbonyl)piperazine-1-carboxylate (23 mg, 0.033 mmol, 10.55% yield) (the longer retention time racemic diastereomer) in pure form as a clear film. As an aside, from this second Combiflash purification an additional quantity of mixed isomers (49 mg, 0.071 mmol, 22.49% yield) was also isolated as a clear film.

rac-Isomer 1: (racemic diastereomer with unknown relative stereochemistry)

LC/MS (ESI): m/z 690.5 (M+H)⁺, 0.98 min (ret. time)

Anal. HPLC: 13.589 min (ret. time). Column: Luna C18 (2) 4.6×150 mm, 3 μm. Mobile Phase: A is Acetonitrile and B is 0.1% TFA in water; 2% B to 95% B gradient over 18 min, then hold at 95% B for 2 min.

rac-Isomer 2: (racemic diastereomer with unknown relative stereochemistry)

LC/MS (ESI): m/z 690.5 (M+H)⁺, 0.98 min (ret. time)

Anal. HPLC: 13.720 min (ret. time). Column: Luna C18 (2) 4.6×150 mm, 3 μm. Mobile Phase: A is Acetonitrile and B is 0.1% TFA in water; 2% B to 95% B gradient over 18 min, hold at 95% B for 2 min.

### Step 5

### Tert-butyl 4-(tert-butyl)-2-(4-((4-chloro-3-fluorophenyl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate

N1-(2,4-dinitrophenyl)-3,6,9,12,15,18,21,24,27-nonaoxanonacosane-1,29-diamine (26 mg, 0.042 mmol) and rac-isomer 2 of tert-butyl 4-(tert-butyl)-2-(4-((4-chloro-3-fluorophenyl)carbamoyl)-3-(2-((phenoxycarbonyl)oxy)ethyl)piperazine-1-carbonyl)piperazine-1-carboxylate (23 mg, 0.033 mmol) were dissolved in 1-butanol (101 μl) and TEA (0.02 mL, 0.13 mmol).

The reaction was heated to 100° C. for 40 min then concentrated onto isolute. The crude product/isolute was purified on a silica cartridge (4 g) with a Combiflash Rf 200i, eluting at 18 mL/min using a non-linear gradient 0-90% (2% NH₄OH in 1:3 EtOH/EtOAc)/hexanes. The desired fractions were concentrated under reduced pressure and dried under high vacuum, giving tert-butyl 4-(tert-butyl)-2-(4-((4-chloro-3-fluorophenyl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate (4 mg, 3.28 μmol, 9.85% yield) as a yellow film [racemic with unknown relative stereochemistry].

LC/MS (ESI): m/z 1218.4 (M+H)⁺, 0.97 min (ret. time).

### Step 6

### 2-(4-(4-(Tert-butyl)piperazine-2-carbonyl)-1-((4-chloro-3-fluorophenyl)carbamoyl)piperazin-2-yl)ethyl (29-((2,4-dinitrophenyl)amino)-3,6,9,12,15,18,21,24,27-nonaoxanonacosyl)carbamate

Tert-butyl 4-(tert-butyl)-2-(4-((4-chloro-3-fluorophenyl)carbamoyl)-3-(34-((2,4-dinitrophenyl)amino)-4-oxo-3,8,11,14,17,20,23,26,29,32-decaoxa-5-azatetratriacontyl)piperazine-1-carbonyl)piperazine-1-carboxylate (4 mg, 3.28 μmol) was dissolved in DCM (0.5 mL) and TFA (0.5 mL, 6.49 mmol). After 30 min of stirring, the reaction was concentrated under reduced pressure then purified on a Gilson HPLC (YMC C18 S-5 μm/12 nm 50×20 mm preparatory column), eluting at 20 mL/min with a linear gradient running from 10% CH₃CN/H₂O (0.1% TFA) to 90% CH₃CN/H₂O (0.1% TFA) over 10 min. The desired fractions were collected and concentrated under reduced pressure resulting in the isolation of 2-(4-(4-(tert-butyl)piperazine-2-carbonyl)-1-((4-chloro-3-fluorophenyl)carbamoyl)piperazin-2-yl)ethyl (29-((2,4-dinitrophenyl)amino)-3,6,9,12,15,18,21,24,27-nonaoxanonacosyl)carbamate, Trifluoroacetic acid salt (2 mg, 1.622 μmol, 49.4% yield) as a yellow film [racemic, single diastereomer with unknown relative stereochemistry].

LC/MS (ESI): m/z 1118.3 (M+H)⁺, 0.89 min (ret. time).

Anal. HPLC: 5.73 min (ret. time) COLUMN: zorbax (agilent)-XDB-C18, 4.6×75 mm, 3.5 um.

Mobile Phase: Solvent A: Water with 0.1% of TFA, and Solvent B: (Acetonitrile with 0.1% of TFA)

GRADIENT: 5-95% B/A over 10 min.

### CCR3

### (S)-4-((3-((4-(3,4-dichlorobenzyl)morpholin-2-yl)methyl)ureido)methyl)-N-(29-((2,4-dinitrophenyl)amino)-3,6,9,12,15,18,21,24,27-nonaoxanonacosyl)benzamide, Trifluoroacetic acid salt

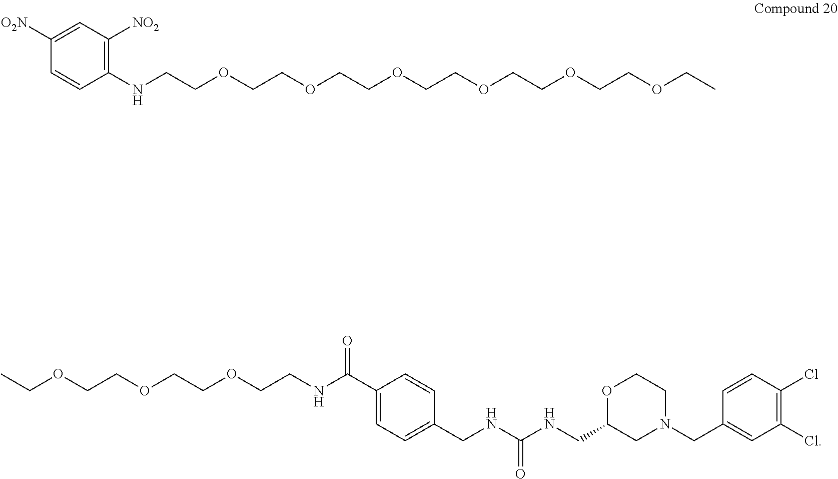

### Step 1

### N1-(2,4-dinitrophenyl)-3,6,9,12,15,18,21,24,27-nonaoxanonacosane-1,29-diamine

A solution of 1-chloro-2,4-dinitrobenzene (2.5 g, 12.34 mmol) and 3,6,9,12,15,18,21,24,27-nonaoxanonacosane-1,29-diamine (11.27 g, 24.69 mmol) in Ethanol (50 mL) was stirred under nitrogen at 20° C. The reaction mixture was stirred at 80° C. for 18h. The solvent was evaporated in vacuo to give the crude product. The crude product was added to a silica gel column and was eluted with DCM/MeOH. Collected fractions: (100/0 to 90/10).

LCMS: 623.0 m/z at 1.237 min

Agilent 1200-6110 Column: Halo C-18, 4.6*50□m Mobile phase: ACN (0.05% FA)/Water (0.05% FA); Gradient:5% ACN to 95% ACN in 1.0 min, hold 1.0 min total 2.5 min Flow rate:1.8 mL/min

### Step 2

### (S)-4-((3-((4-(3,4-dichlorobenzyl)morpholin-2-yl)methyl)ureido)methyl)benzoic acid

In a 100 mL round bottom flask, (S)-4-((3-((4-(3,4-dichlorobenzyl)morpholin-2-yl)methyl)ureido)methyl)benzamide benzenesulfonate dihydrate (3 g, 4.65 mmol) (WO2002026723A1) and LiOH (1.669 g, 69.7 mmol) were suspended in water (12 mL), methanol (12 mL), and 1,4-Dioxane (12 mL). A water condenser was affixed to the flask, and the mixture heated at 100° C. (external) for 70 h. The basic solution was acidified with AcOH and added to an SCX-3 cartridge (10 g). The column was washed with MeOH then eluted with NH₃ (2N in MeOH). The eluant was concentrated under a stream of nitrogen at 50° C. resulting in (S)-4-((3-((4-(3,4-dichlorobenzyl)morpholin-2-yl)methyl)ureido)methyl)benzoic acid (410 mg, 0.906 mmol, 19.50% yield) as a white solid.

LC/MS (ESI): m/z 452.0 (M+H)⁺; 0.56 min (ret. time)

### Step 3

### (S)-4-((3-((4-(3,4-dichlorobenzyl)morpholin-2-yl)methyl)ureido)methyl)-N-(29-((2,4-dinitrophenyl)amino)-3,6,9,12,15,18,21,24,27-nonaoxanonacosyl)benzamide, Trifluoroacetic acid salt

(S)-4-((3-((4-(3,4-dichlorobenzyl)morpholin-2-yl)methyl)ureido)methyl)benzoic acid (16.3 mg, 0.036 mmol) and TSTU (11.9 mg, 0.040 mmol) were dissolved in DMF (0.4 mL) and TEA (10 μl, 0.072 mmol). After 45 min of stirring, N1-(2,4-dinitrophenyl)-3,6,9,12,15,18,21,24,27-nonaoxanonacosane-1,29-diamine (24.68 mg, 0.040 mmol) dissolved in DMF (0.4 mL) was added. After 2.5 h of stirring, the reaction was diluted with water and purified on a x-bridge prep C18 5 um OBD 30×150 mm column with a gradient from 20-100% ACN/(0.1% NH₄OH/H₂O) over 15 min. The desired fractions were collected and concentrated under a stream of nitrogen at 50° C. resulting in a yellow film (18 mg). The film was dissolved in water/MeOH and purified on a Gilson HPLC (Sunfire 5 μm C18 OBD 19×100 mm preparatory column), eluting at 20 mL/min with a linear gradient running from 10-90% CH₃CN/H₂O (0.1% TFA) over 12 min. The desired fractions were concentrated, resulting in the production of (S)-4-((3-((4-(3,4-dichlorobenzyl)morpholin-2-yl)methyl)ureido)methyl)-N-(29-((2,4-dinitrophenyl)amino)-3,6,9,12,15,18,21,24,27-nonaoxanonacosyl)benzamide, Trifluoroacetic acid salt (10 mg, 8.54 μmol, 23.70% yield) as a yellow solid. LC/MS (ESI): m/z 529.4 (M+2H)⁺⁺, 0.95 min (ret. time)

¹H NMR (400 MHz, METHANOL-d₄) δ ppm 2.91 (t, J=11.54 Hz, 1H) 3.10-3.22 (m, 1H) 3.28 (m, 1H) 3.36-3.45 (m, 2H) 3.54-3.86 (m, 43H) 4.13 (dd, J=12.92, 2.64 Hz, 1H) 4.37 (s, 4H) 7.23 (d, J=9.54 Hz, 1H) 7.38 (d, J=8.03 Hz, 2H) 7.45 (d, J=8.28 Hz, 1H) 7.67 (d, J=8.28 Hz, 1H) 7.75 (s, 1H) 7.81 (d, J=8.03 Hz, 2H) 8.29 (dd, J=9.54, 1.76 Hz, 1H) 9.03 (d, J=1.76 Hz, 1H). Long LCMS (12 min): 6.676 min (ret. time); Luna C18 (2) 4.6×150 mm, 3 u. 2-95% (0.1% TFA in ACN)/water over 18 min, held 95% for 2 min.

### (R)-4-((3-((4-(3,4-dichlorobenzyl)morpholin-2-yl)methyl)ureido)methyl)-N-(3-(2-(2-(3-((2,4-dinitrophenyl)amino)propoxy)ethoxy)ethoxy)propyl)benzamide

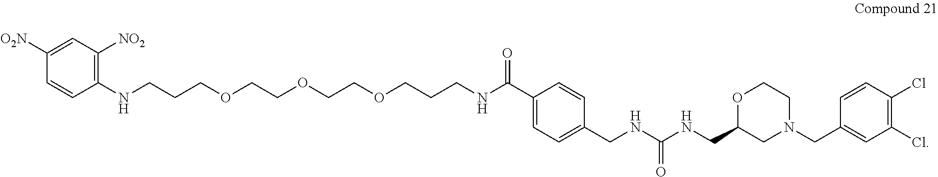 [SMILES: O=C(NCc1ccc(C(=O)NCCCOCCOCCOCCCNc2ccc([N+](=O)[O-])cc2[N+](=O)[O-])cc1)NC[C@@H]1CN(Cc2ccc(Cl)c(Cl)c2)CCO1]

### Step 1

### N-(3-(2-(2-(3-aminopropoxy)ethoxy)ethoxy)propyl)-2,4-dinitroaniline

In a 20 mL microwave vial 1-chloro-2,4-dinitrobenzene (9.24 g, 45.6 mmol) was dissolved in ethanol (152 ml). 3,3′-((oxybis(ethane-2,1-diyl))bis(oxy))bis(propan-1-amine) (10 ml, 45.6 mmol) was added by syringe and the reaction was heated to reflux for 2.5 h with a water condensor affixed. After the reaction cooled to room temperature it was concentrated under reduced pressure. The residue was dissolved in DCM, isolute was added, and the mixture was concentrated under reduced pressure. The crude product was purified on a silica cartridge (330 g) with a Combiflash torrent, eluting at 100 mL/min with a non-linear 0-15% MeOH/DCM gradient. The desired fractions were concentrated under reduced pressure and dried under high vacuum, giving N-(3-(2-(2-(3-aminopropoxy)ethoxy)ethoxy)propyl)-2,4-dinitroaniline (4.85 g, 12.55 mmol) as a viscous orange oil.

LCMS: m/z 387.5 [M+H]+, 0.69 min (ret. time)

### Step 2

### (R)-4-((3-((4-(3,4-dichlorobenzyl)morpholin-2-yl)methyl)ureido)methyl)-N-(3-(2-(2-(3-((2,4-dinitrophenyl)amino)propoxy)ethoxy)ethoxy)propyl)benzamide

(S)-4-((3-((4-(3,4-dichlorobenzyl)morpholin-2-yl)methyl)ureido)methyl)benzoic acid (38 mg, 0.084 mmol) (from Example 15 step 3) and TSTU (27.8 mg, 0.092 mmol) were dissolved in DMSO (0.4 mL) and TEA (0.05 mL, 0.36 mmol) for 9 min via sonication. N-(3-(2-(2-(3-aminopropoxy)ethoxy)ethoxy)propyl)-2,4-dinitroaniline (42.6 mg, 0.101 mmol) was dissolved in DMSO (0.4 mL) and added to the reaction vial. The reaction was sonicated for 30 min. Then purified on a Gilson HPLC Waters (Sunfire 20×100 mm) with a linear gradient running from 20-50% CH₃CN/H₂O (0.1% TFA) over 10 min to afford (R)-4-((3-((4-(3,4-dichlorobenzyl)morpholin-2-yl)methyl)ureido)methyl)-N-(3-(2-(2-(3-((2,4-dinitrophenyl)amino)propoxy)ethoxy)ethoxy)propyl)benzamide. LC/MS (ESI): m/z 820.1 (M+H)⁺, 0.82 min (ret. time)¹H NMR (400 MHz, METHANOL-d₄) δ ppm 1.87 (t, J=6.27 Hz, 2H) 1.93-2.04 (m, 2H) 2.84-2.98 (m, 1H) 3.05-3.22 (m, 1H) 3.37-3.51 (m, 4H) 3.53-3.89 (m, 17H) 4.07-4.19 (m, 1H) 4.36 (s, 4H) 7.17 (d, J=9.54 Hz, 1H) 7.35 (d, J=8.03 Hz, 2H) 7.44 (d, J=8.03 Hz, 1H) 7.67 (d, J=8.28 Hz, 1H) 7.71-7.78 (m, 3H) 8.26 (d, J=9.54 Hz, 1H) 8.99 (s, 1H) HPLC: 12.198 min (ret. time) on a Luna C18 (2) 4.6×150 mm, 3 μm. 2% B to 95% B in 18 min, hold at 95% B for 2 min, Acidic Conditions. 0.1% TFA in ACN (% B) and H2O (% A).

### (S)-5-(3-(1-(4-((3-((4-(3,4-Dichlorobenzyl)morpholin-2-yl)methyl)ureido)methyl)phenyl)-1-oxo-6,9,12-trioxa-2-azapentadecan-15-yl)thioureido)-2-(6-hydroxy-3-oxo-3H-xanthen-9-yl)benzoic acid, Trifluoroacetic acid salt

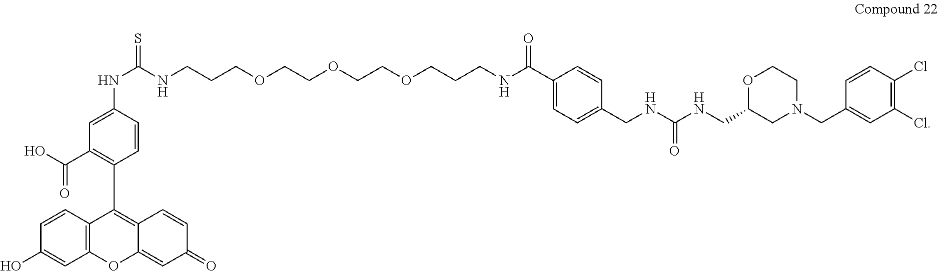 [SMILES: O=C(NCc1ccc(C(=O)NCCCOCCOCCOCCCNC(=S)Nc2ccc(-c3c4ccc(=O)cc-4oc4cc(O)ccc34)c(C(=O)O)c2)cc1)NC[C@H]1CN(Cc2ccc(Cl)c(Cl)c2)CCO1]

### Step 1

### 1-(3-(2-(2-(3-aminopropoxy)ethoxy)ethoxy)propyl)-3-(3′,6′-dihydroxy-3-oxo-3H-spiro[isobenzofuran-1,9′-xanthen]-5-yl)thiourea, Trifluoroacetic acid salt

To 3,3′-((oxybis(ethane-2,1-diyl))bis(oxy))bis(propan-1-amine) (3.39 g, 15.4 mmol) dissolved in Methanol (10 ml) was added 3′,6′-dihydroxy-5-isothiocyanato-3H-spiro[isobenzofuran-1,9′-xanthen]-3-one (3.0 g, 7.7 mmol) in small portions. A mild exotherm was observed and the solution immediately became a dark red. After 15 min the reaction was transferred to a test tube using methanol and purified on a Gilson HPLC (x-bridge prep C18 10 um OBD 50×250 mm column) with a linear gradient of 10-75% ACN/(0.1% TFA/H₂O) over 40 min. The desired fractions were collected and concentrated under reduced pressure, using toluene, acetonitrile, and heptane as azeotroping reagents. This resulted in an orange/red solid after drying under high vacuum, 1-(3-(2-(2-(3-aminopropoxy)ethoxy)ethoxy)propyl)-3-(3′,6′-dihydroxy-3-oxo-3H-spiro[isobenzofuran-1,9′-xanthen]-5-yl)thiourea, Trifluoroacetic acid salt (3.59 g, 4.96 mmol, 64.4% yield). LC/MS (ESI): m/z 610.2 (M+H)⁺, 0.65 min (ret. time)

### Step 2

### (S)-5-(3-(1-(4-((3-((4-(3,4-dichlorobenzyl)morpholin-2-yl)methyl)ureido)methyl)phenyl)-1-oxo-6,9,12-trioxa-2-azapentadecan-15-yl)thioureido)-2-(6-hydroxy-3-oxo-3H-xanthen-9-yl)benzoic acid, Trifluoroacetic acid salt

(S)-4-((3-((4-(3,4-dichlorobenzyl)morpholin-2-yl)methyl)ureido)methyl)benzoic acid (38 mg, 0.084 mmol) and TSTU (27.8 mg, 0.092 mmol) were dissolved in DMSO (0.4 mL) and TSTU (27.8 mg, 0.092 mmol). The reaction was sonicated for 15 min then 5-((3-(2-(2-(3-aminopropoxy)ethoxy)ethoxy)propyl)amino)-2-(6-hydroxy-3-oxo-3H-xanthen-9-yl)benzoic acid, Trifluoroacetic acid salt (67.0 mg, 0.101 mmol) was added followed by DMSO (0.4 mL) and TEA (0.05 mL, 0.36 mmol). The reaction was stirred overnight at room temperature. The reaction was transferred to a test tube with AcOH and the crude product was purified on a Gilson HPLC (Sunfire 5 μm C18 OBD 30×100 mm preparatory column), eluting at 30 mL/min with a linear gradient running from 10-90% CH₃CN/H₂O (0.1% TFA) over 10 min. The desired fractions were lyophilized, giving (S)-5-(3-(1-(4-((3-((4-(3,4-dichlorobenzyl)morpholin-2-yl)methyl)ureido)methyl)phenyl)-1-oxo-6,9,12-trioxa-2-azapentadecan-15-yl)thioureido)-2-(6-hydroxy-3-oxo-3H-xanthen-9-yl)benzoic acid, Trifluoroacetic acid salt (11 mg, 9.50 μmol, 11.31% yield) as a light orange solid. LC/MS (ESI): m/z 522.4 (M+2H)⁺⁺, 0.79 min (ret. time)

¹H NMR (400 MHz, ACETONITRILE-d₃ and D₂O) δ ppm 1.70-1.89 (m, 4H) 2.69-2.87 (m, 1H) 2.93-3.09 (m, 1H) 3.38 (br. s., 6H) 3.55 (br. s., 12H) 3.69-3.81 (m, 2H) 3.96-4.11 (m, 1H) 4.18-4.32 (m, 4H) 6.60 (br. s., 2H) 6.71 (d, J=18.82 Hz, 4H) 7.06-7.17 (m, 1H) 7.33 (br. s., 3H) 7.71 (d, J=7.28 Hz, 5H) 8.07-8.16 (m, 1H)

HPLC: 11.157 min (ret. time) on a Luna C18 (2) 4.6×150 mm column, 3 μm, 2% B to 95% B; 18 min, hold at 95% B for 2 min, Acidic Conditions. 0.1% TFA in ACN (% B) and H₂O (% A).

### N1-((Trans)-4-(4-((3-(((S)-4-(3,4-Dichlorobenzyl)morpholin-2-yl)methyl)ureido)methyl)benzamido)cyclohexyl)-N4-(3-(2-(2-(3-((2,4-dinitrophenyl)amino)propoxy)ethoxy)ethoxy)propyl)succinamide, Trifluoroacetic acid salt

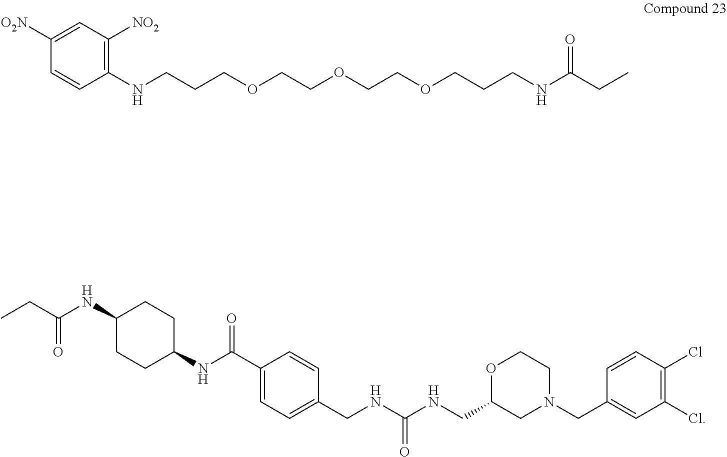

### Step 1

### Tert-butyl ((trans)-4-(1-((2,4-dinitrophenyl)amino)-15-oxo-4,7,10-trioxa-14-azaoctadecanamido)cyclohexyl)carbamate

Tert-butyl ((trans)-4-aminocyclohexyl)carbamate (1.29 g, 6.02 mmol) (from Example 16 step 1) and 1-((2,4-dinitrophenyl)amino)-15-oxo-4,7,10-trioxa-14-azaoctadecan-18-oic acid (2.1 g, 4.32 mmol) were dissolved in DCM (33 mL). 2,4,6-tripropyl-1,3,5,2,4,6-trioxatriphosphinane 2,4,6-trioxide (50 wt % in EtOAc) (5.5 ml, 9.24 mmol) was added in one portion. The reaction was stirred overnight (18 h). A gelatinous material formed in the flask. The reaction was diluted with DCM and partitioned with saturated sodium bicarbonate. An emulsion formed between two layers. The DCM layer was taken and the emulsion/aq layers extracted twice with DCM. The combined DCM layers were washed with brine (2nd emulsion). DCM layer taken again, brine and emulsion extracted with DCM. Combined DCM was concentrated under reduced pressure onto isolute. The crude product was purified on a silica cartridge (80 g, gold) with a Combiflash Rf 200i, eluting at 60 mL/min with a non-linear 0-100% Acetone/hexanes gradient. The desired fractions were concentrated under reduced pressure and dried under high vacuum, giving tert-butyl ((trans)-4-(1-((2,4-dinitrophenyl)amino)-15-oxo-4,7,10-trioxa-14-azaoctadecanamido)cyclohexyl)carbamate (597 mg, 0.874 mmol, 20.26% yield) as a yellow solid. LC/MS (ESI): m/z 683.6 (M+H)⁺, 1.06 min (ret. time)

### Step 2

### N1-((Trans)-4-aminocyclohexyl)-N4-(3-(2-(2-(3-((2,4-dinitrophenyl)amino)propoxy)ethoxy)ethoxy)propyl)succinamide

Tert-butyl ((trans)-4-(1-((2,4-dinitrophenyl)amino)-15-oxo-4,7,10-trioxa-14-azaoctadecanamido)cyclohexyl)carbamate (597 mg, 0.874 mmol) was dissolved in HCl (4 N in Dioxane) (7.5 mL, 30.0 mmol). After stirring for 30 min at room temperature the reaction was concentrated under a stream of nitrogen at 50° C. then under high vacuum, resulting in N1-((1r,4r)-4-aminocyclohexyl)-N4-(3-(2-(2-(3-((2,4-dinitrophenyl)amino)propoxy)ethoxy)ethoxy)propyl)succinamide, Hydrochloride (488 mg, 0.788 mmol, 90% yield) as a yellow solid. LC/MS (ESI): m/z 583.5 (M+H)⁺, 0.76 min (ret. time)

### Step 3

### N1-((1S,4r)-4-(4-((3-(((S)-4-(3,4-dichlorobenzyl)morpholin-2-yl)methyl)ureido)methyl)benzamido)cyclohexyl)-N4-(3-(2-(2-(3-((2,4-dinitrophenyl)amino)propoxy)ethoxy)ethoxy)propyl)succinamide, Trifluoroacetic acid salt

(S)-4-((3-((4-(3,4-dichlorobenzyl)morpholin-2-yl)methyl)ureido)methyl)benzoic acid (34 mg, 0.075 mmol) and TSTU (24.89 mg, 0.083 mmol) were dissolved in DMSO (0.4 mL) and TEA (0.01 mL, 0.072 mmol) and sonicated for 15 min. N1-((trans)-4-aminocyclohexyl)-N4-(3-(2-(2-(3-((2,4-dinitrophenyl)amino)propoxy)ethoxy)ethoxy)propyl)succinamide, Hydrochloride (51.2 mg, 0.083 mmol) (solid) was added, and the reaction diluted with DMSO (0.4 mL). After 30 min of sonication the reaction was purified on a Gilson HPLC (Waters Sunfire 30×150 mm), eluting at 50 mL/min with a linear gradient running from 20-60% CH₃CN/H₂O (0.1% TFA) over 26 min resulting in N1-((1S,4r)-4-(4-((3-(((S)-4-(3,4-dichlorobenzyl)morpholin-2-yl)methyl)ureido)methyl)benzamido)cyclohexyl)-N4-(3-(2-(2-(3-((2,4-dinitrophenyl)amino)propoxy)ethoxy)ethoxy)propyl)succinamide, Trifluoroacetic acid salt (30 mg, 0.027 mmol, 35.3% yield) as a yellow solid. LC/MS (ESI): m/z 508.8 (M+2H)⁺⁺, 1.74 min (ret. time).

### (2S,3S)—N-(1-(4-((3-(((S)-4-(3,4-dichlorobenzyl)morpholin-2-yl)methyl)ureido)methyl)phenyl)-1-oxo-5,8,11,14,17,20,23,26,29-nonaoxa-2-azahentriacontan-31-yl)-1-methyl-5-oxo-2-(pyridin-3-yl)pyrrolidine-3-carboxamide, Trifluoroacetic acid salt

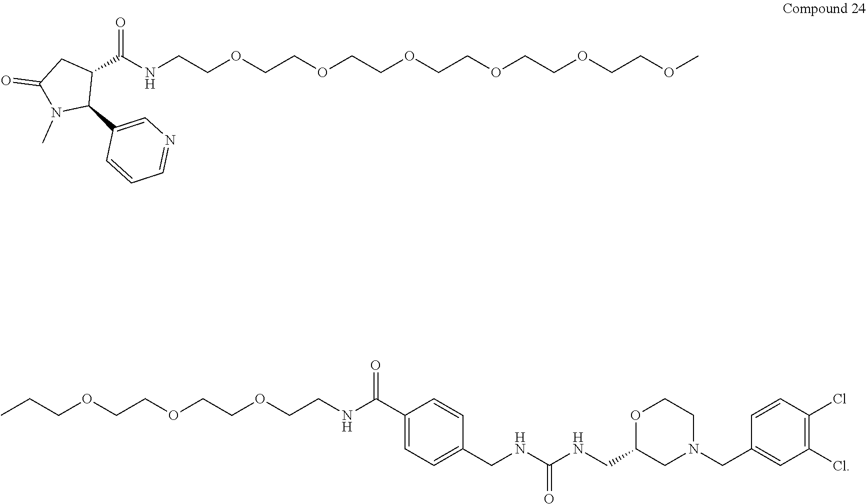

### Step 1

### (2S,3S)—N-(29-amino-3,6,9,12,15,18,21,24,27-nonaoxanonacosyl)-1-methyl-5-oxo-2-(pyridin-3-yl)pyrrolidine-3-carboxamide

(2S,3S)-1-Methyl-5-oxo-2-(pyridin-3-yl)pyrrolidine-3-carboxylic acid (96 mg, 0.44 mmol) and TSTU (135 mg, 0.448 mmol) were dissolved in DMSO (4.4 mL) and DIPEA (0.17 mL, 0.973 mmol). After 15 minutes of sonication, 3,6,9,12,15,18,21,24,27-nonaoxanonacosane-1,29-diamine (259 mg, 0.567 mmol) dissolved in DMSO (1.0 mL) was added. The reaction was sonicated a further 15 min, then purified (2 injections) on a x-bridge prep C18 5 Dm OBD 30×150 mm column with a gradient from 10-50% ACN/(0.1% NH₄OH/H₂O) over 10 min. The desired fractions were collected and concentrated under a stream of nitrogen at 50° C. resulting in the production of (2S,3S)—N-(29-amino-3,6,9,12,15,18,21,24,27-nonaoxanonacosyl)-1-methyl-5-oxo-2-(pyridin-3-yl)pyrrolidine-3-carboxamide (83 mg, 0.126 mmol, 28.9% yield) as a clear oil. LC/MS (ESI): m/z 521.4 (M+H)⁺, 0.66 min (ret. time)

### Step 2

### (2S,3S)—N-(1-(4-((3-(((S)-4-(3,4-dichlorobenzyl)morpholin-2-yl)methyl)ureido)methyl)phenyl)-1-oxo-5,8,11,14,17,20,23,26,29-nonaoxa-2-azahentriacontan-31-yl)-1-methyl-5-oxo-2-(pyridin-3-yl)pyrrolidine-3-carboxamide, Trifluoroacetic acid salt

(S)-4-((3-((4-(3,4-dichlorobenzyl)morpholin-2-yl)methyl)ureido)methyl)benzoic acid (34.5 mg, 0.076 mmol) and TSTU (25.3 mg, 0.084 mmol) were dissolved in DMSO (0.4 mL) and TEA (0.1 mL, 0.717 mmol). The reaction was sonicated for 30 min then (2S,3S)—N-(29-amino-3,6,9,12,15,18,21,24,27-nonaoxanonacosyl)-1-methyl-5-oxo-2-(pyridin-3-yl)pyrrolidine-3-carboxamide (709 μl, 0.084 mmol) was added, and the reaction was sonicated for a further 30 min. The crude product was transferred to a test tube with AcOH and purified (two injections) on a Gilson HPLC (Sunfire 5 μm C18 OBD 19×100 mm preparatory column), eluting at 20 mL/min with a linear gradient running from 10-50% CH₃CN/H₂O (0.1% TFA) over 10 min. The desired fractions were concentrated followed by exposure to high vacuum, resulting in the production of (2S,3S)—N-(1-(4-((3-(((S)-4-(3,4-dichlorobenzyl)morpholin-2-yl)methyl)ureido)methyl)phenyl)-1-oxo-5,8,11,14,17,20,23,26,29-nonaoxa-2-azahentriacontan-31-yl)-1-methyl-5-oxo-2-(pyridin-3-yl)pyrrolidine-3-carboxamide, Trifluoroacetic acid salt (60 mg, 0.050 mmol, 65.2% yield) as a clear film. LC/MS (ESI): m/z 546.8 (M+2H)+⁺, 0.54 min (ret. time) cacl'd M+2H++546.75.

HPLC: 8.506 min (ret. time) on a Luna C18 (2) 4.6×150 mm, 3μ. 2% B to 95% B in 18 min, hold at 95% B for 2 min, Acidic Condition. 0.1% TFA in ACN (% B) and H₂O (% A)

¹H NMR (400 MHz, METHANOL-d₄) δ ppm 2.68-2.96 (m, 6H) 3.00-3.10 (m, 1H) 3.11-3.21 (m, 1H) 3.29 (m, 1H) 3.36-3.71 (m, 44H) 3.79 (m, 3H) 4.07-4.19 (m, 1H) 4.38 (s, 4H) 4.95 (d, J=6.78 Hz, 1H) 7.39 (d, J=7.78 Hz, 2H) 7.46 (s, 1H) 7.68 (d, J=8.28 Hz, 1H) 7.76 (s, 1H) 7.82 (d, J=7.78 Hz, 2H) 7.94-8.05 (m, 1H) 8.30-8.38 (m, 1H) 8.78-8.86 (m, 2H).

### CCR5

### 4-(4-(((R)-1-butyl-3-((R)-cyclohexyl(hydroxy)methyl)-2,5-dioxo-1,4,9-triazaspiro[5.5]undecan-9-yl)methyl)phenoxy)-N-(1-((3′,6′-dihydroxy-3-oxo-3H-spiro[isobenzofuran-1,9′-xanthen]-5-yl)amino)-1-thioxo-5,8,11,14,17,20,23,26,29-nonaoxa-2-azahentriacontan-31-yl)benzamide, trifluoroacetic acid salt

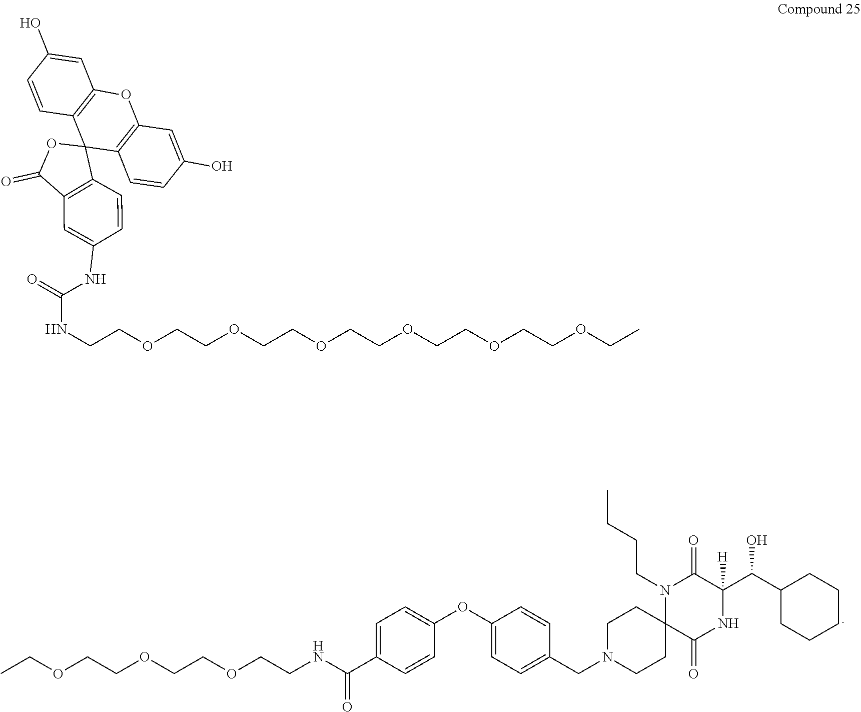

### Step 1

### Tert-butyl (1-(4-(4-(((R)-1-butyl-3-((R)-cyclohexyl(hydroxy)methyl)-2,5-dioxo-1,4,9-triazaspiro[5.5]undecan-9-yl)methyl)phenoxy)phenyl)-1-oxo-5,8,11,14,17,20,23,26,29-nonaoxa-2-azahentriacontan-31-yl)carbamate

To a mixture of tert-butyl (29-amino-3,6,9,12,15,18,21,24,27-nonaoxanonacosyl)carbamate (176 mg, 0.316 mmol), 4-(4-(((R)-1-butyl-3-((R)-cyclohexyl(hydroxy)methyl)-2,5-dioxo-1,4,9-triazaspiro[5.5]undecan-9-yl)methyl)phenoxy)benzoic acid (183 mg, 0.316 mmol) and HATU (144 mg, 0.379 mmol) in Dichloromethane (DCM) was added DIPEA (221 μl, 1.265 mmol). The mixture was stirred at rt overnight. The mixture was distributed between EtOAc and water, extracted with EtOAc. The combined organics were washed with brine, dried over Na₂SO₄, and filtered. The filtrate was concentrated.

The product was was purified via silica gel chromatography (Biotage Isolera, 40 μm. Redisep Gold column, 0˜20% MeOH (2N NH₃)/DCM, loaded as a solution in DCM) to give tert-butyl (1-(4-(4-(((R)-1-butyl-3-((R)-cyclohexyl(hydroxy)methyl)-2,5-dioxo-1,4,9-triazaspiro[5.5]undecan-9-yl)methyl)phenoxy)phenyl)-1-oxo-5,8,11,14,17,20,23,26,29-nonaoxa-2-azahentriacontan-31-yl)carbamate (340 mg, 0.305 mmol, 96% yield) as a colorless oil. ¹H NMR (400 MHz, METHANOL-d₄) □ ppm 0.87-2.34 (m, 35H) 3.18-3.86 (m, 46H) 4.17 (d, J=2.26 Hz, 1H) 7.08 (dd, J=16.06, 8.53 Hz, 4H) 7.46 (d, J=8.28 Hz, 2H) 7.83-7.92 (m, 2H). LC-MS: m/z 559.3 (½M+1).

### Step 2

### N-(29-amino-3,6,9,12,15,18,21,24,27-nonaoxanonacosyl)-4-(4-(((R)-1-butyl-3-((R)-cyclohexyl(hydroxy)methyl)-2,5-dioxo-1,4,9-triazaspiro[5.5]undecan-9-yl)methyl)phenoxy)benzamide, 2Trifluoroacetic acid salt

To a mixture of tert-butyl (1-(4-(4-(((R)-1-butyl-3-((R)-cyclohexyl(hydroxy)methyl)-2,5-dioxo-1,4,9-triazaspiro[5.5]undecan-9-yl)methyl)phenoxy)phenyl)-1-oxo-5,8,11,14,17,20,23,26,29-nonaoxa-2-azahentriacontan-31-yl)carbamate (310 mg, 0.278 mmol) in Dichloromethane (DCM) (1 mL) was added TFA (0.321 mL, 4.17 mmol). The mixture was stirred at rt overnight. The mixture was concentrated to dryness to give N-(29-amino-3,6,9,12,15,18,21,24,27-nonaoxanonacosyl)-4-(4-(((R)-1-butyl-3-((R)-cyclohexyl(hydroxy)methyl)-2,5-dioxo-1,4,9-triazaspiro[5.5]undecan-9-yl)methyl)phenoxy)benzamide, 2Trifluoroacetic acid salt (340 mg, 0.273 mmol, 98% yield) as a colorless oil. The product was used in the next step without further purification. LC-MS: m/z 509.3 (½M+1).

### Step 3

### 4-(4-(((R)-1-butyl-3-((R)-cyclohexyl(hydroxy)methyl)-2,5-dioxo-1,4,9-triazaspiro[5.5]undecan-9-yl)methyl)phenoxy)-N-(1-((3′,6′-dihydroxy-3-oxo-3H-spiro[isobenzofuran-1,9′-xanthen]-5-yl)amino)-1-thioxo-5,8,11,14,17,20,23,26,29-nonaoxa-2-azahentriacontan-31-yl)benzamide, trifluoroacetic acid salt

To a mixture of N-(29-amino-3,6,9,12,15,18,21,24,27-nonaoxanonacosyl)-4-(4-(((R)-1-butyl-3-((R)-cyclohexyl(hydroxy)methyl)-2,5-dioxo-1,4,9-triazaspiro[5.5]undecan-9-yl)methyl)phenoxy)benzamide, 2Trifluoroacetic acid salt (38.2 mg, 0.031 mmol) in Dichloromethane (DCM) (0.5 mL) and N,N-Dimethylformamide (DMF) (0.500 mL), was added 3′,6′-dihydroxy-5-isothiocyanato-3H-spiro[isobenzofuran-1,9′-xanthen]-3-one (11.95 mg, 0.031 mmol) and TEA (0.026 mL, 0.183 mmol). The mixture was stirred at rt for 2h. The mixture was concentrated. The product was purified via reverse-phase chromatography (Gilson Autoprep, 25˜55% MeCN/water with 0.1% TFA, acidic Luna column) to give 4-(4-(((R)-1-butyl-3-((R)-cyclohexyl(hydroxy)methyl)-2,5-dioxo-1,4,9-triazaspiro[5.5]undecan-9-yl)methyl)phenoxy)-N-(1-((3′,6′-dihydroxy-3-oxo-3H-spiro[isobenzofuran-1,9′-xanthen]-5-yl)amino)-1-thioxo-5,8,11,14,17,20,23,26,29-nonaoxa-2-azahentriacontan-31-yl)benzamide, trifluoroacetic acid salt. ¹H NMR (400 MHz, METHANOL-d₄) □ ppm 0.80-2.59 (m, 26H) 3.01-4.44 (m, 46H) 6.53-6.62 (m, 2H) 6.66-6.78 (m, 4H) 7.04-7.12 (m, 2H) 7.13-7.24 (m, 3H) 7.51-7.62 (m, 2H) 7.77-7.84 (m, 1H) 7.85-7.93 (m, 2H) 8.22-8.31 (m, 1H). LC-MS: m/z 704.1 (½M+1).

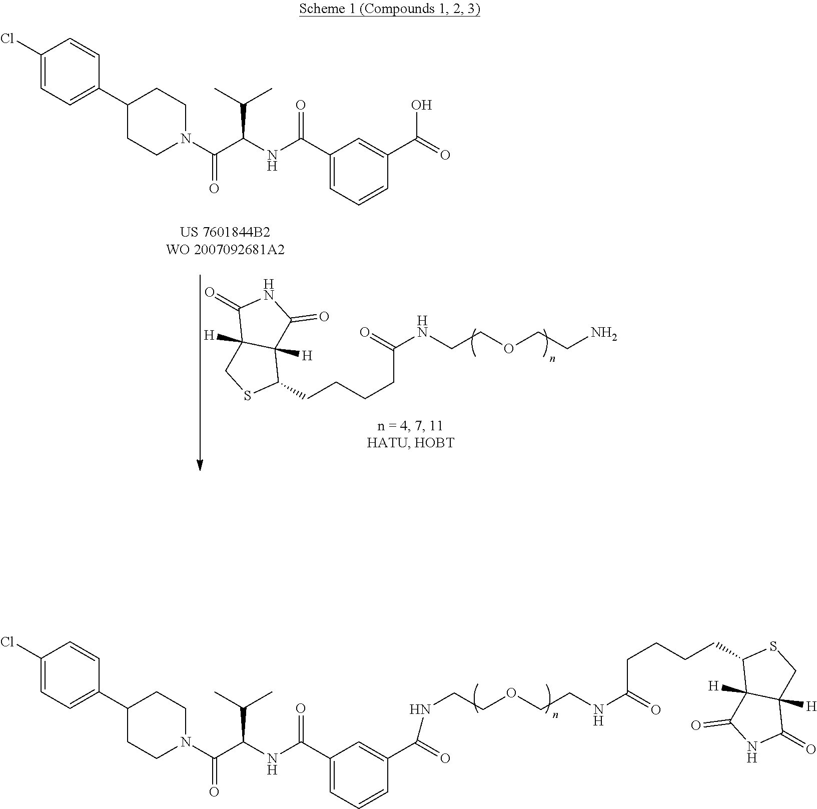

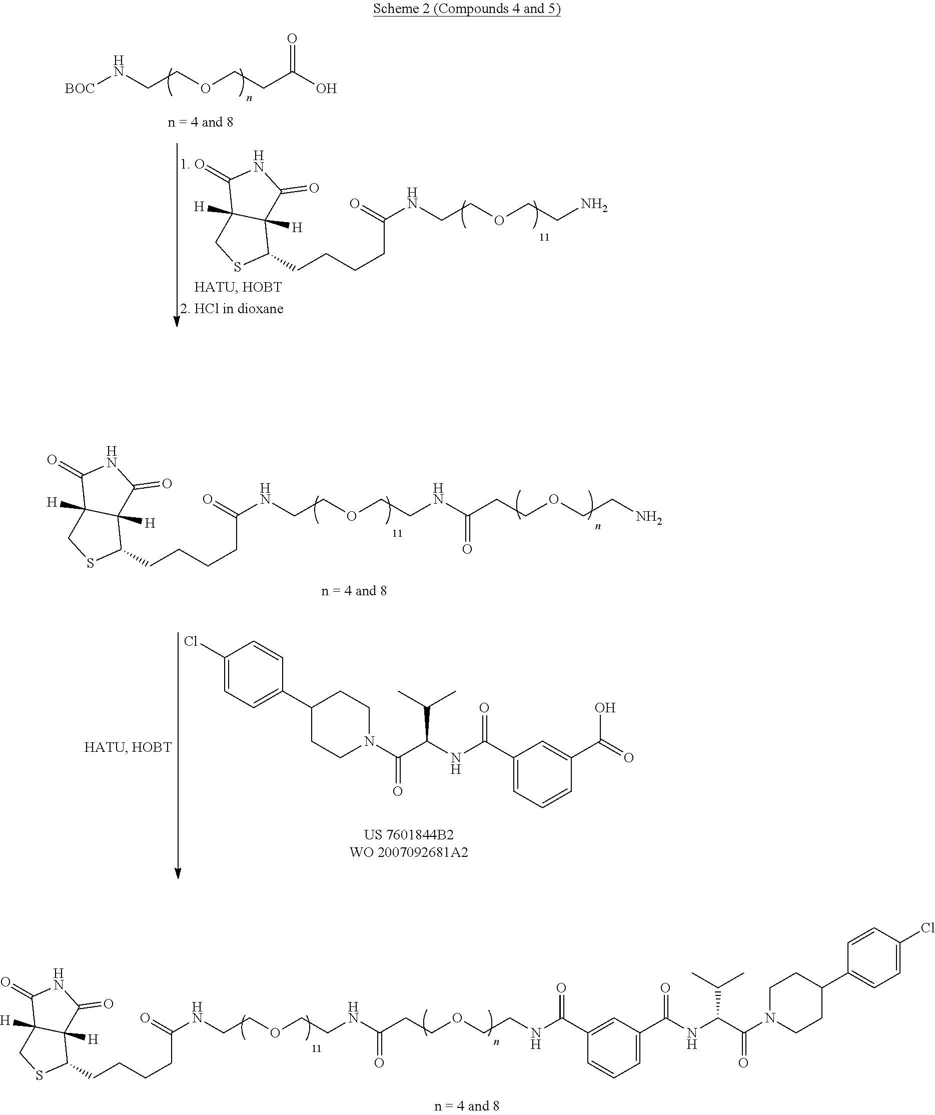

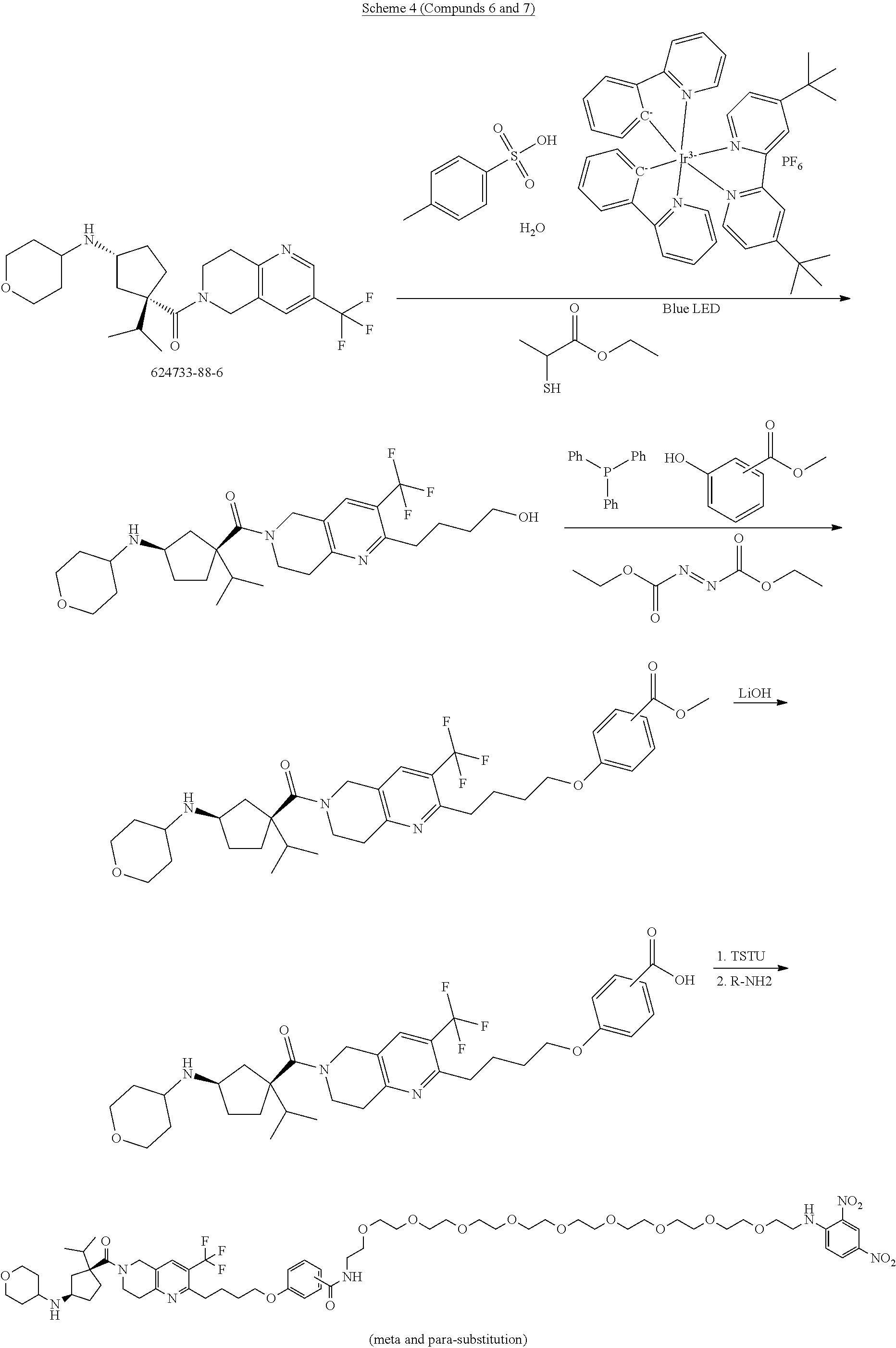

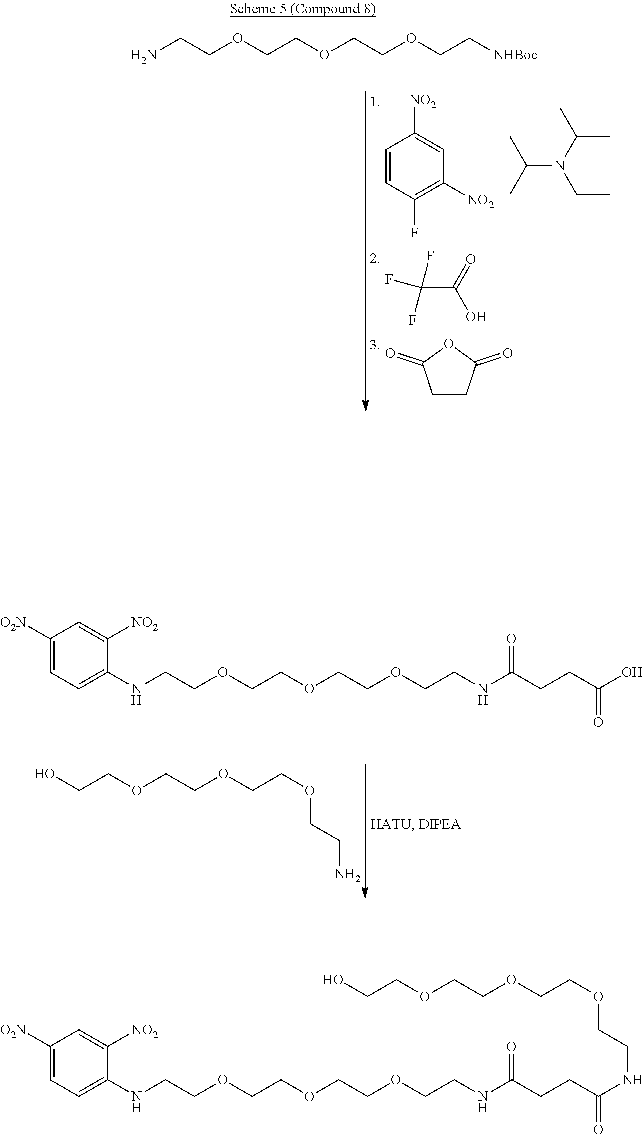

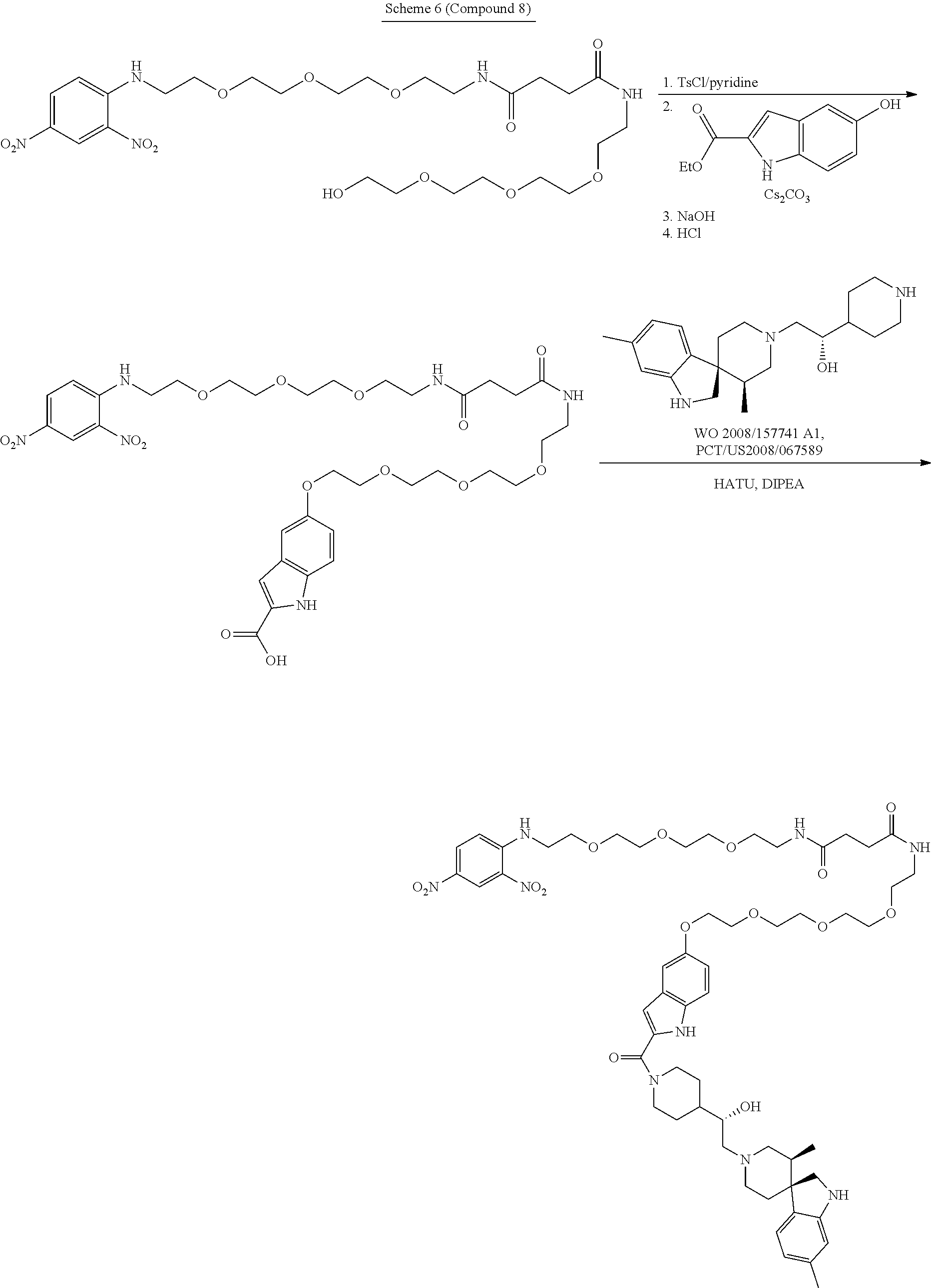

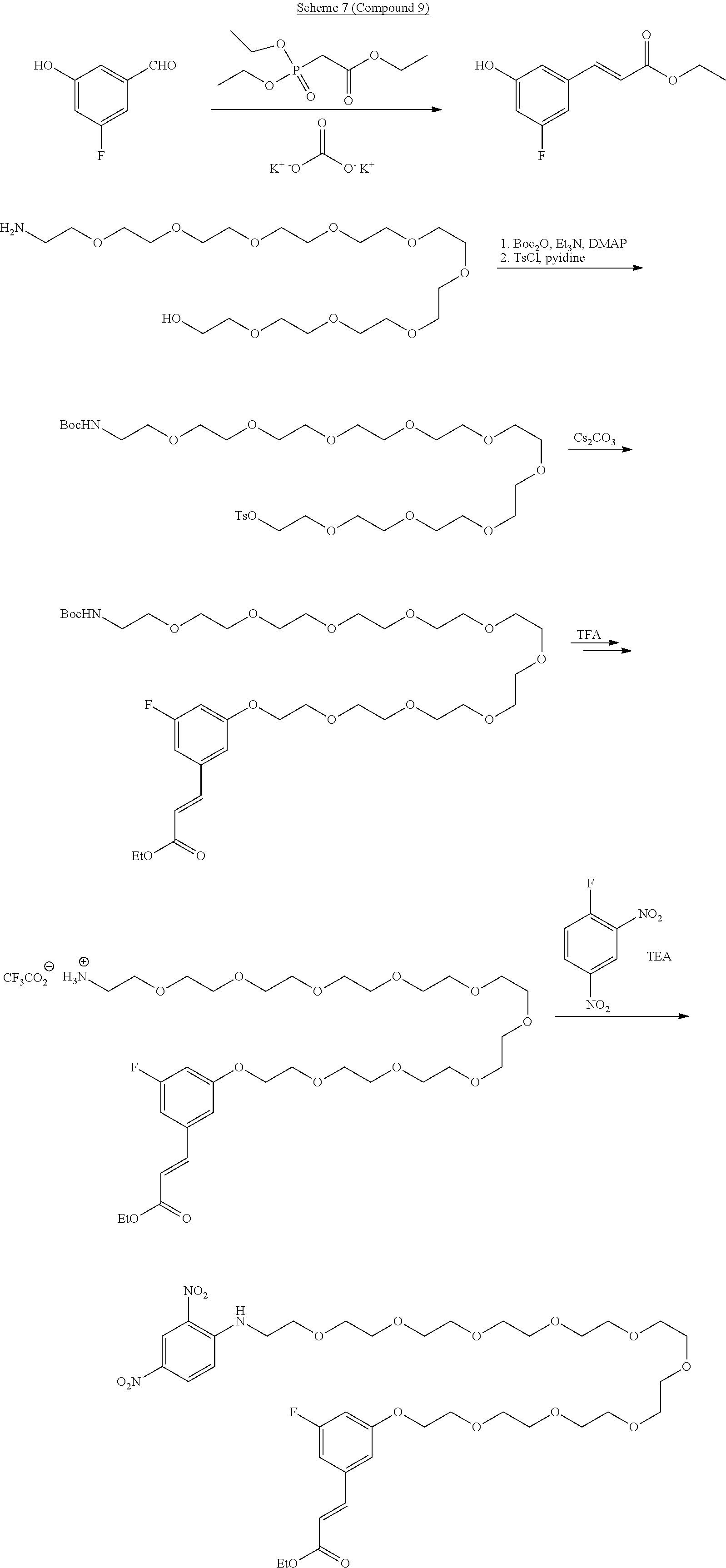

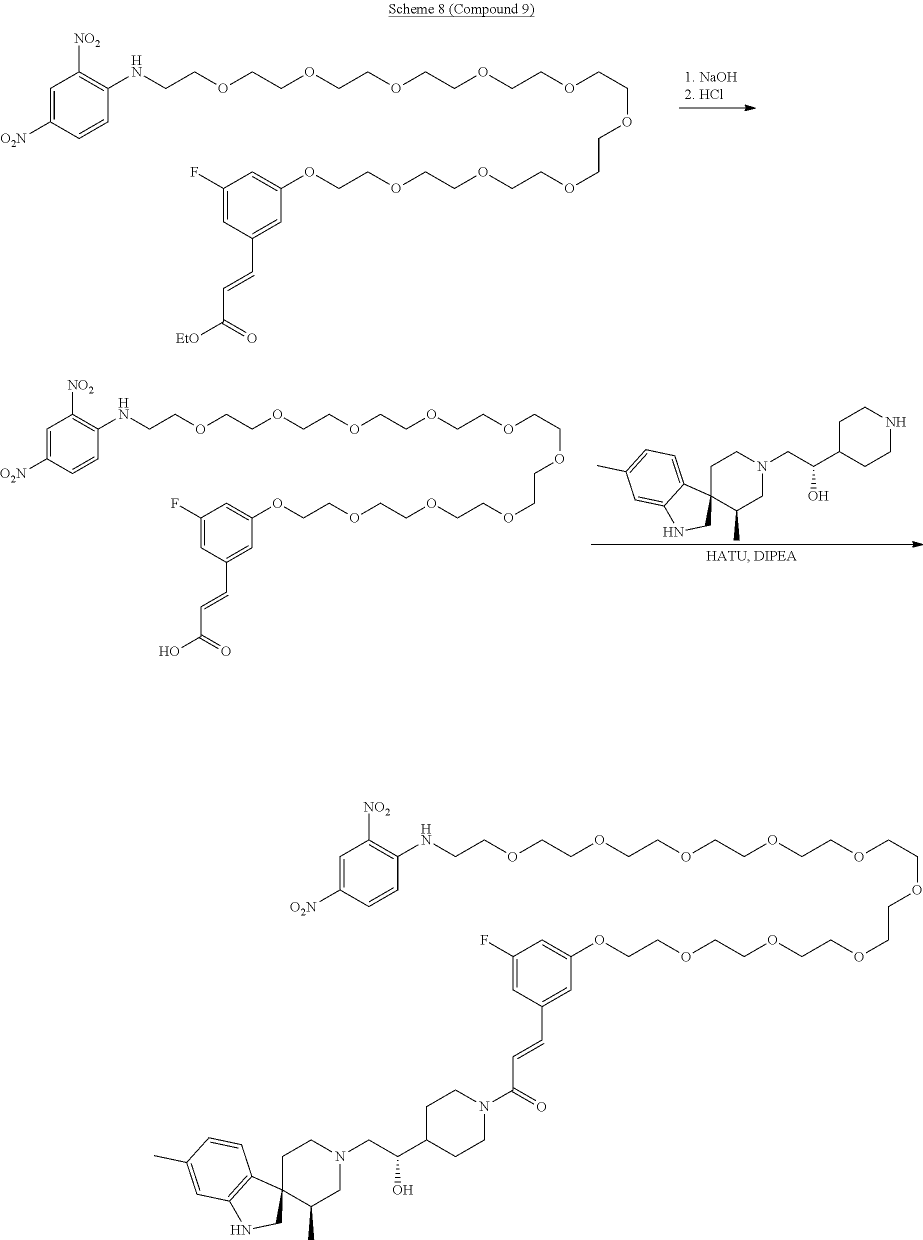

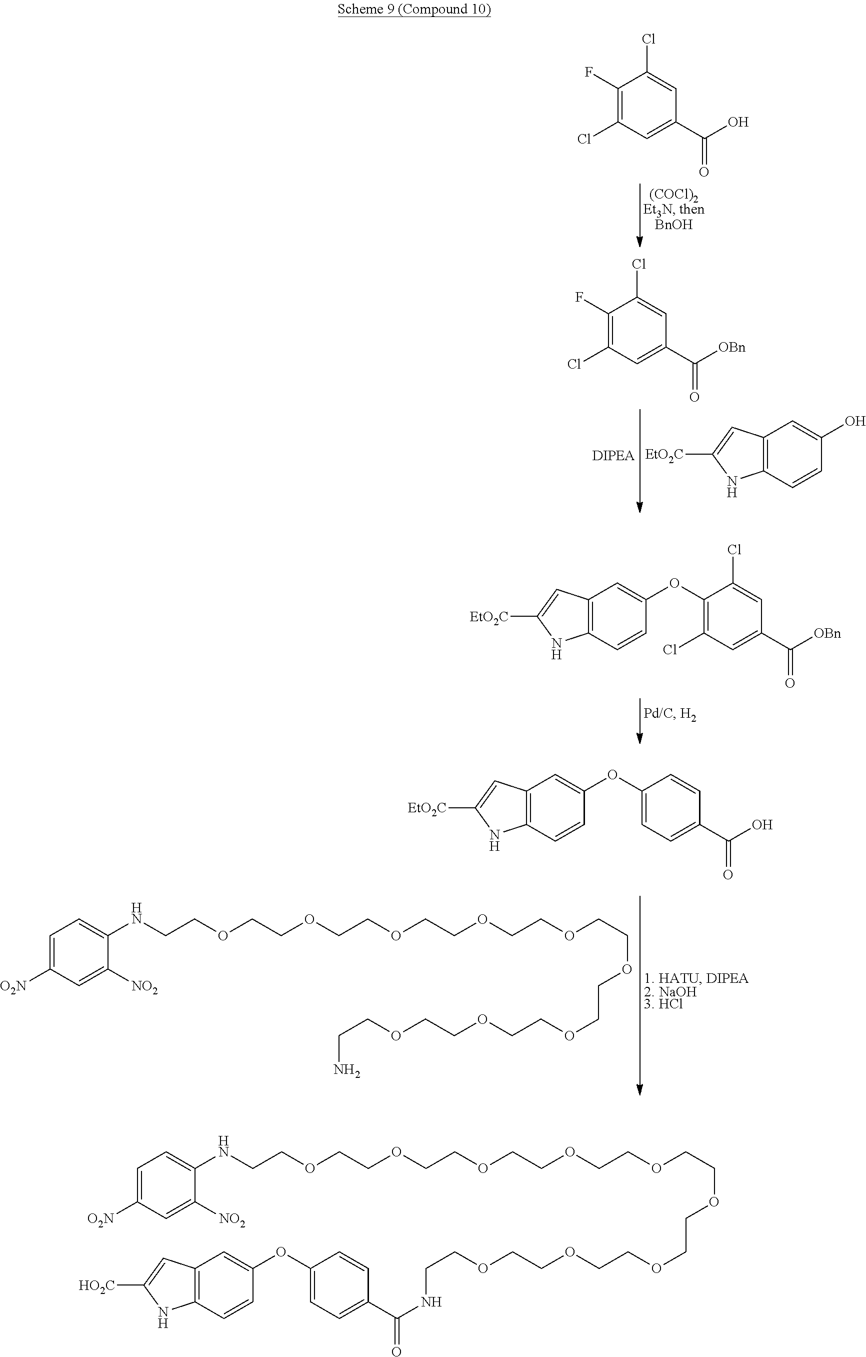

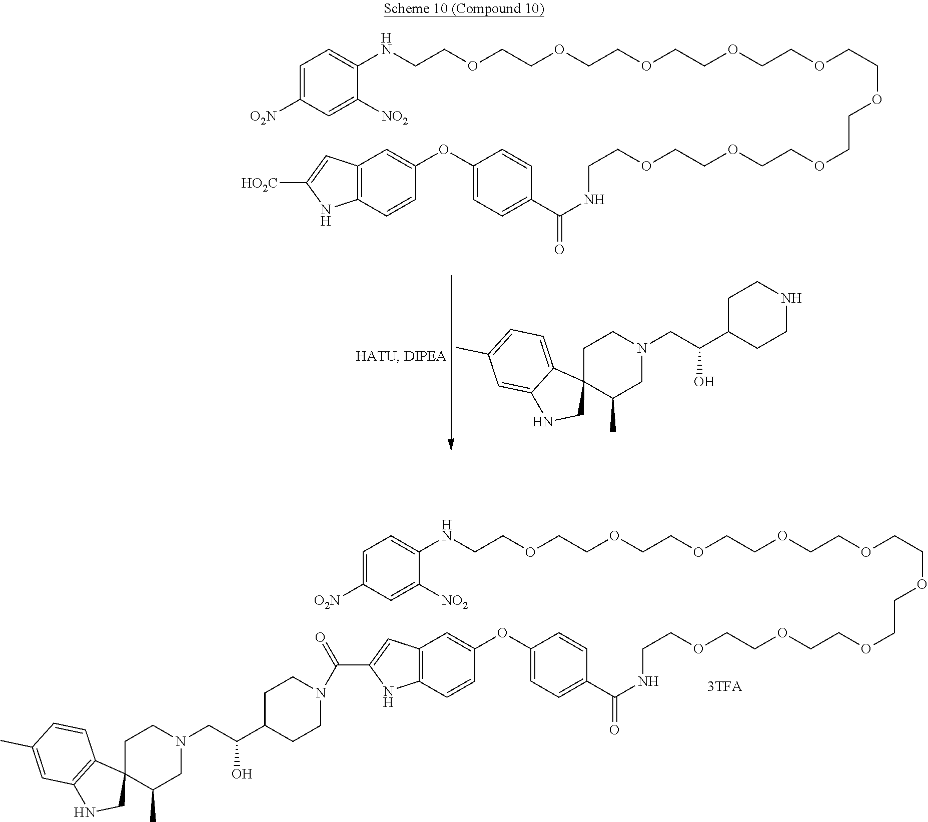 [SMILES: O=C(NCCOCCOCCOCCOCCOCCOCCOCCOCCOCCNc1ccc([N+](=O)[O-])cc1[N+](=O)[O-])c1ccc(Oc2ccc3[nH]c(C(=O)O)cc3c2)cc1 ; Cc1ccc2c(c1)NC[C@]21CCN(C[C@@H](O)C2CCNCC2)C[C@H]1C ; Cc1ccc2c(c1)NC[C@]21CCN(C[C@@H](O)C2CCN(C(=O)c3cc4cc(Oc5ccc(C(=O)NCCOCCOCCOCCOCCOCCOCCOCCOCCOCCNc6ccc([N+](=O)[O-])cc6[N+](=O)[O-])cc5)ccc4[nH]3)CC2)C[C@H]1C]

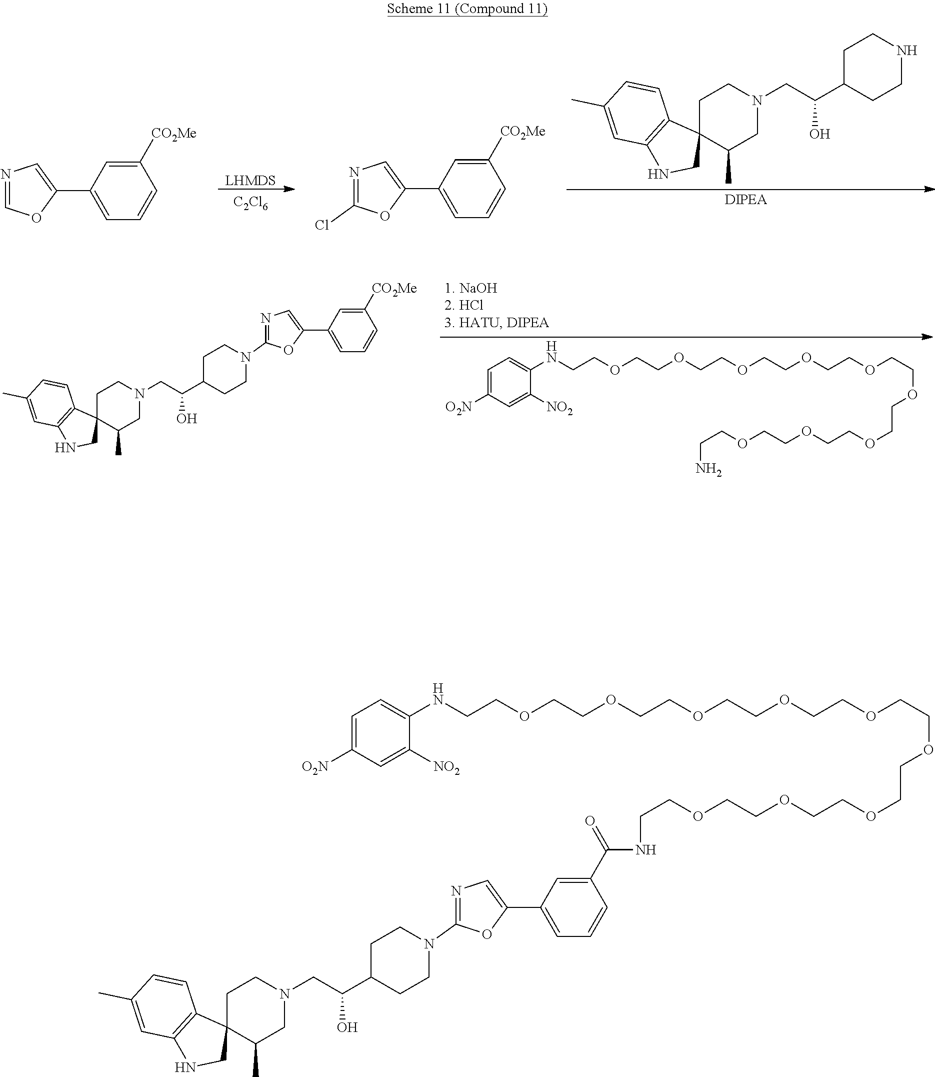

### Example 12 (Compound 12)

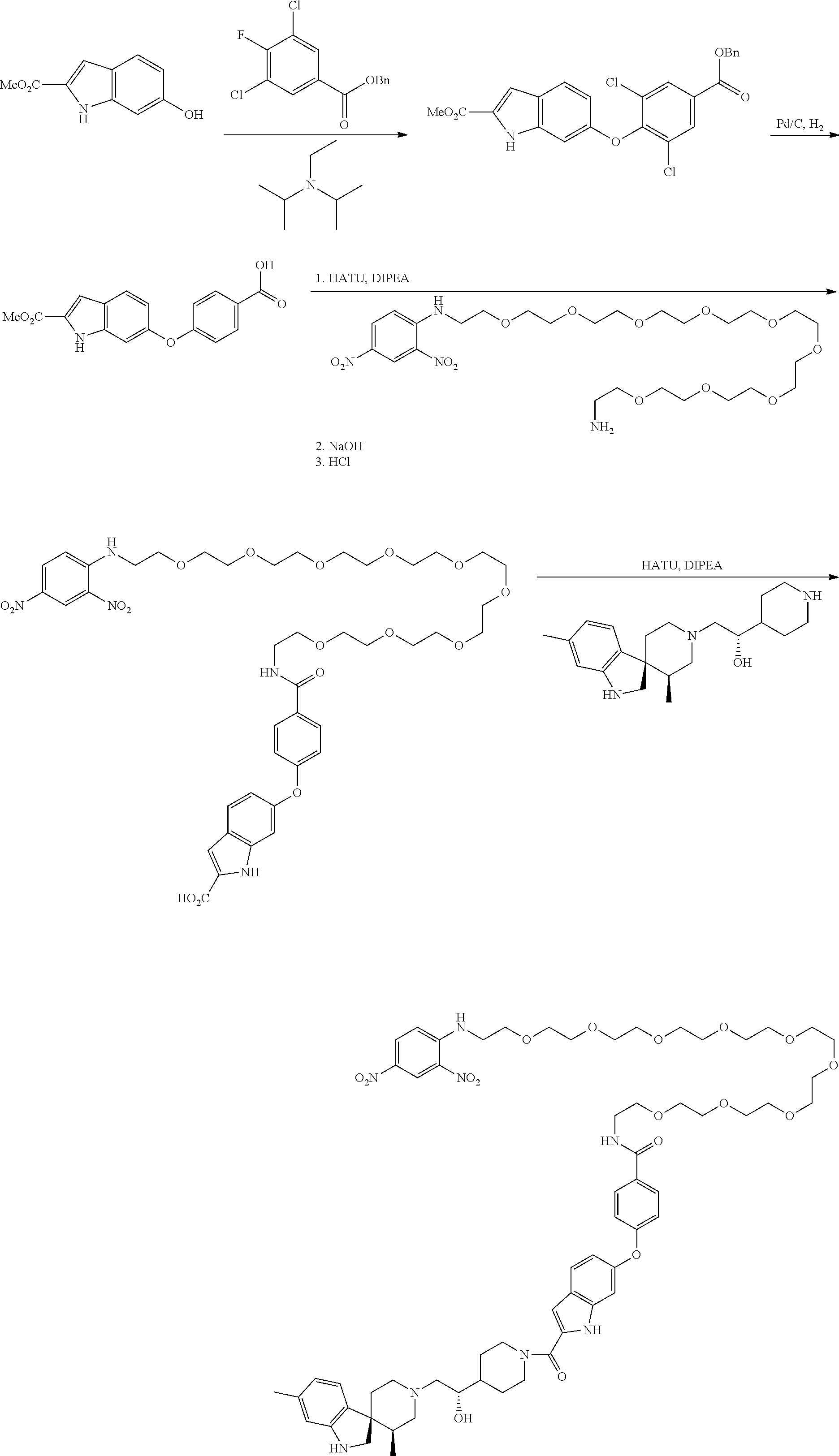

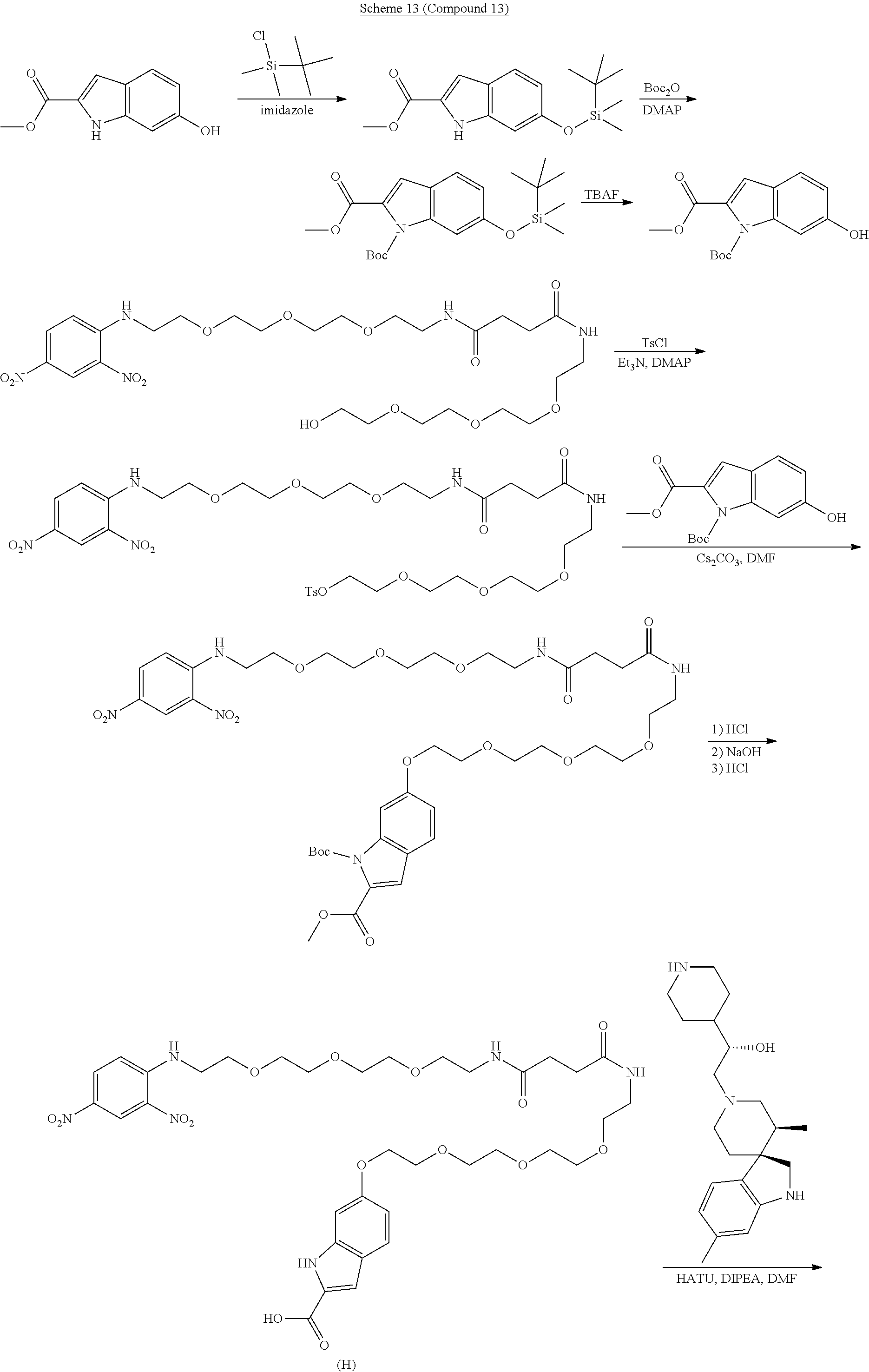

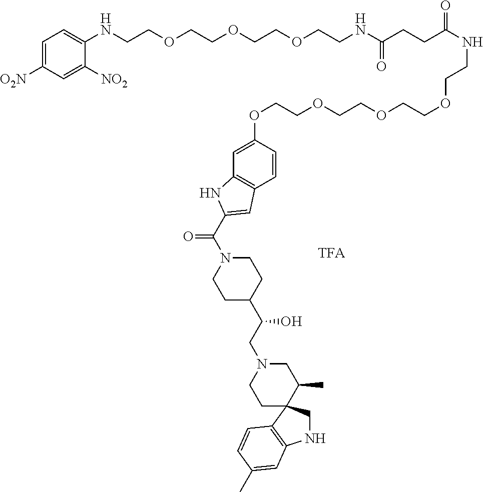 [SMILES: Cc1ccc2c(c1)NC[C@]21CCN(C[C@@H](O)C2CCN(C(=O)c3cc4ccc(OCCOCCOCCOCCNC(=O)CCC(=O)NCCOCCOCCOCCNc5ccc([N+](=O)[O-])cc5[N+](=O)[O-])cc4[nH]3)CC2)C[C@H]1C]

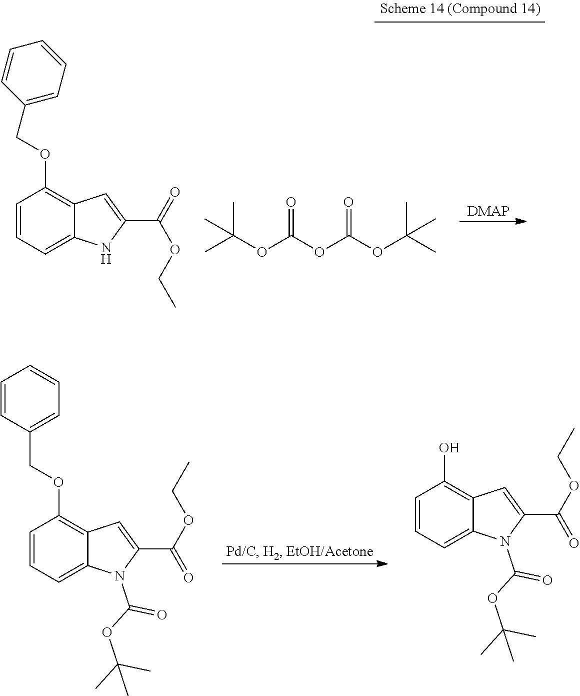

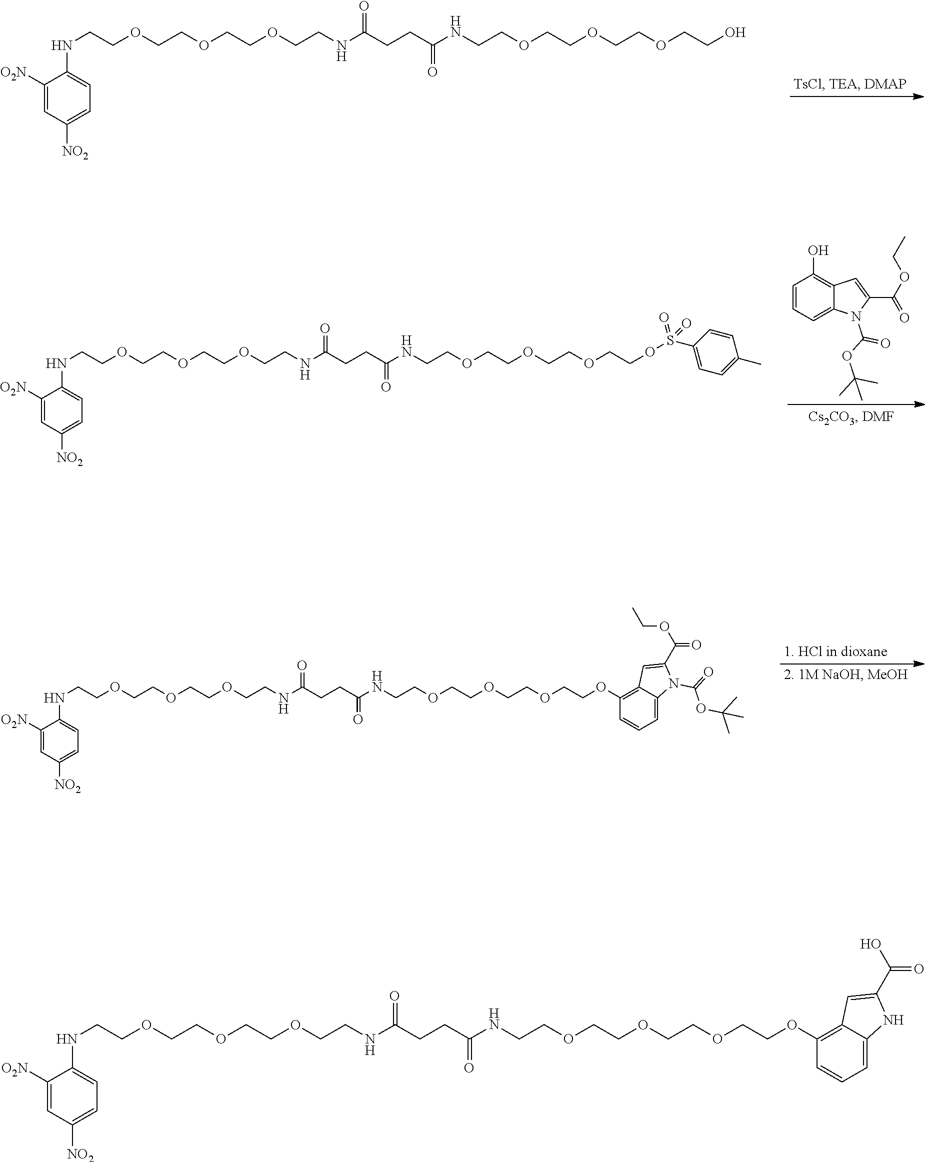

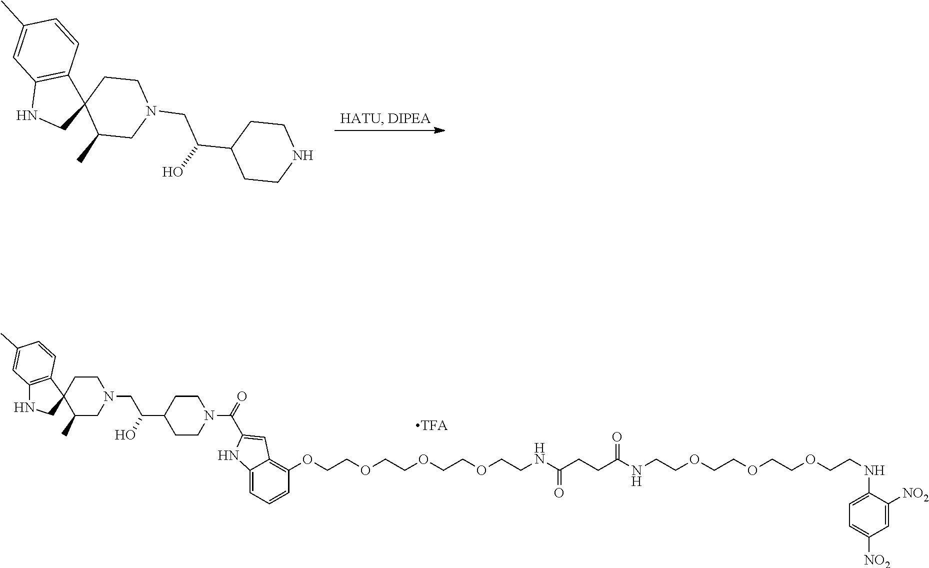 [SMILES: Cc1ccc2c(c1)NC[C@]21CCN(C[C@@H](O)C2CCNCC2)C[C@H]1C ; Cc1ccc2c(c1)NC[C@]21CCN(C[C@@H](O)C2CCN(C(=O)c3cc4c(OCCOCCOCCOCCNC(=O)CCC(=O)NCCOCCOCCOCCNc5ccc([N+](=O)[O-])cc5[N+](=O)[O-])cccc4[nH]3)CC2)C[C@H]1C]

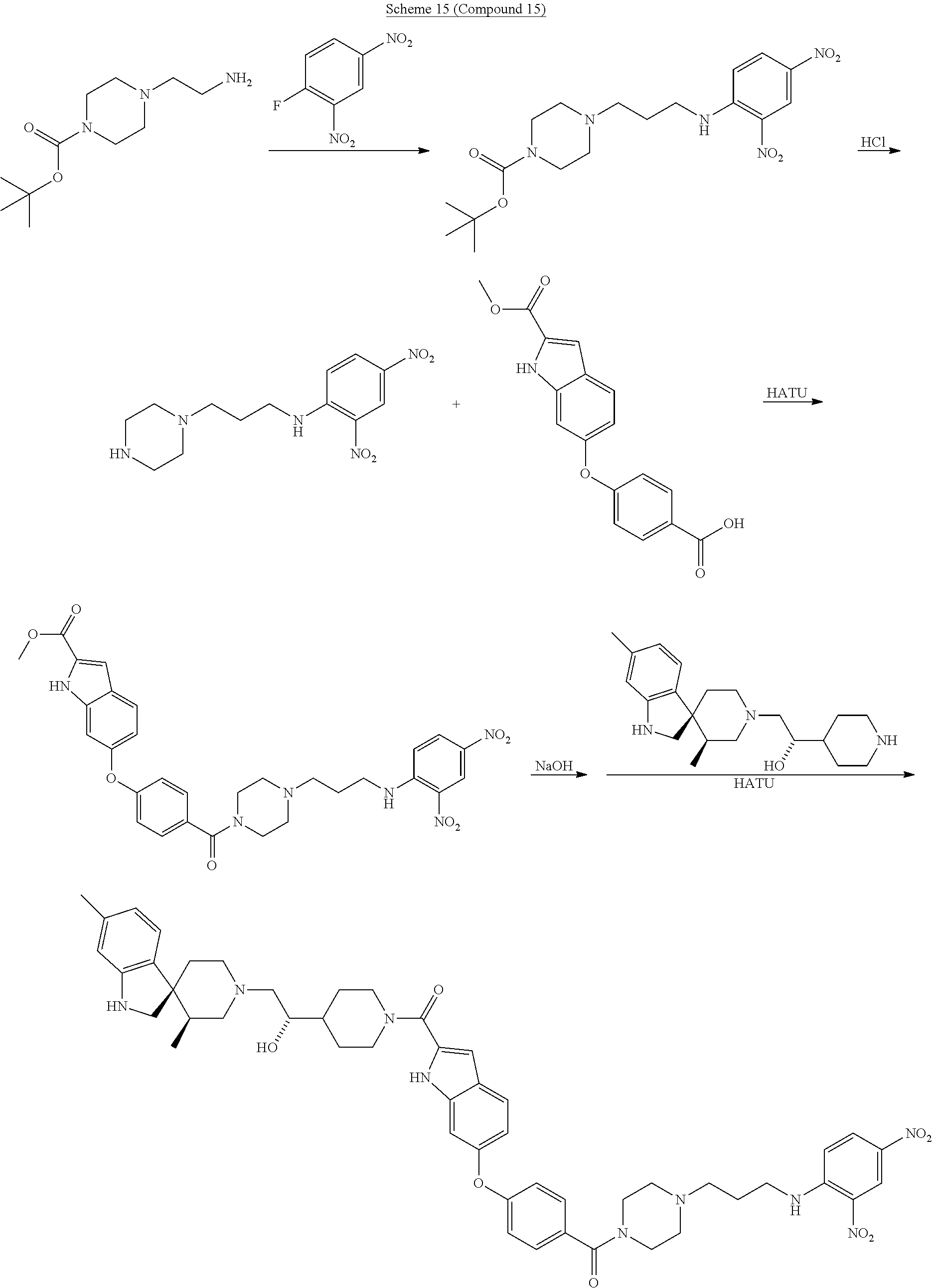

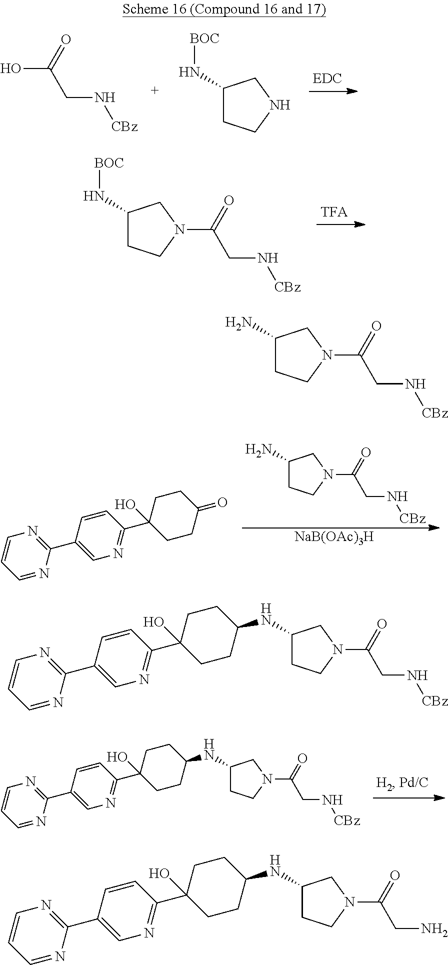

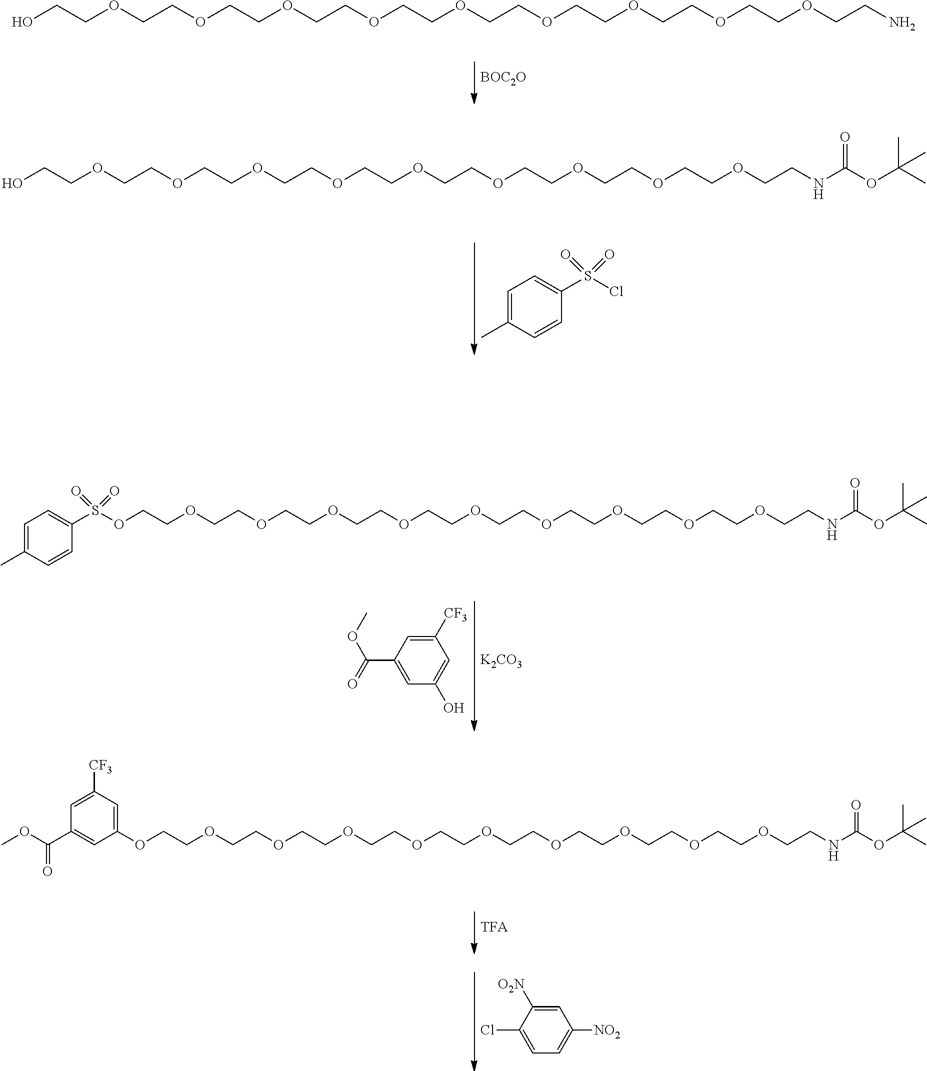

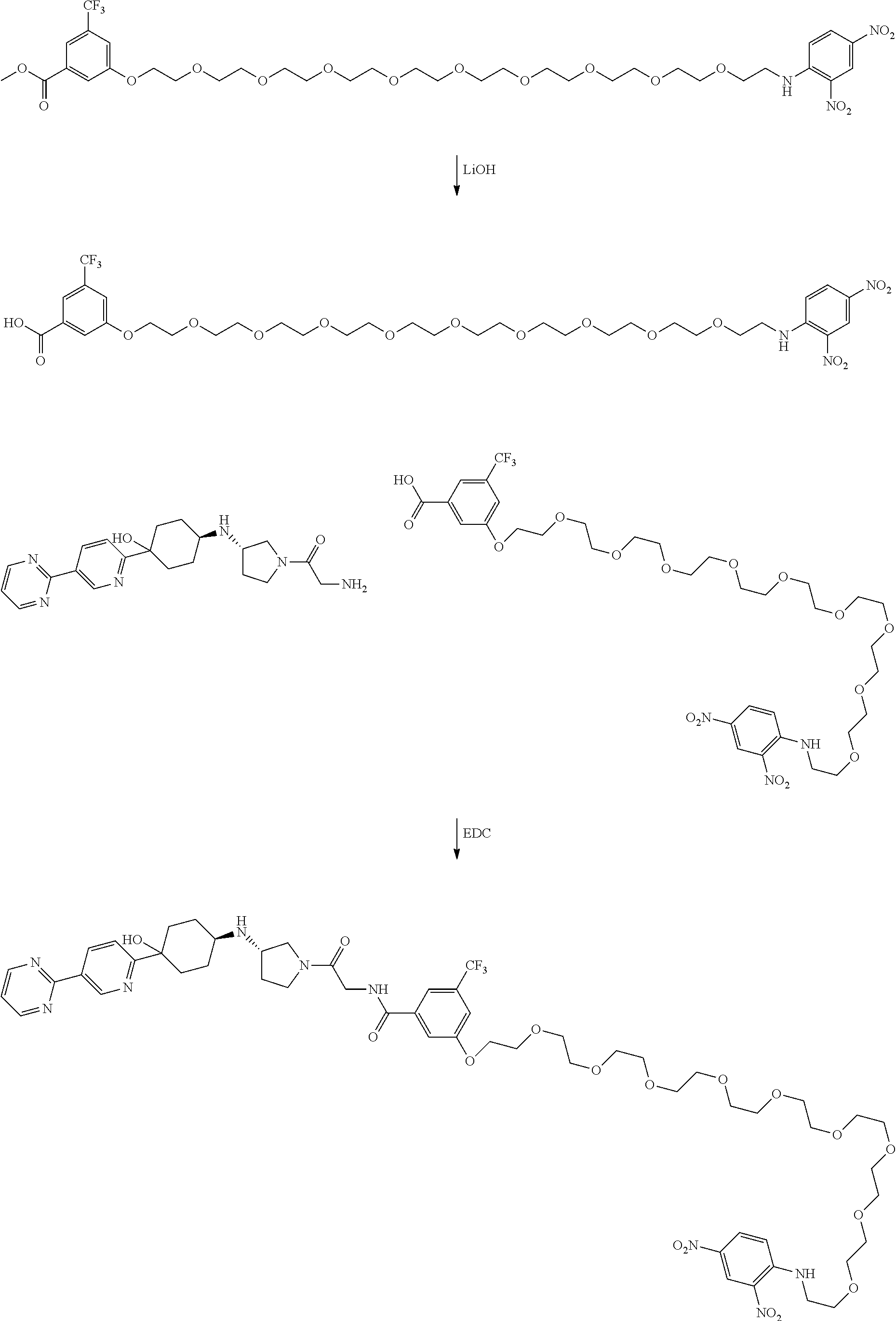

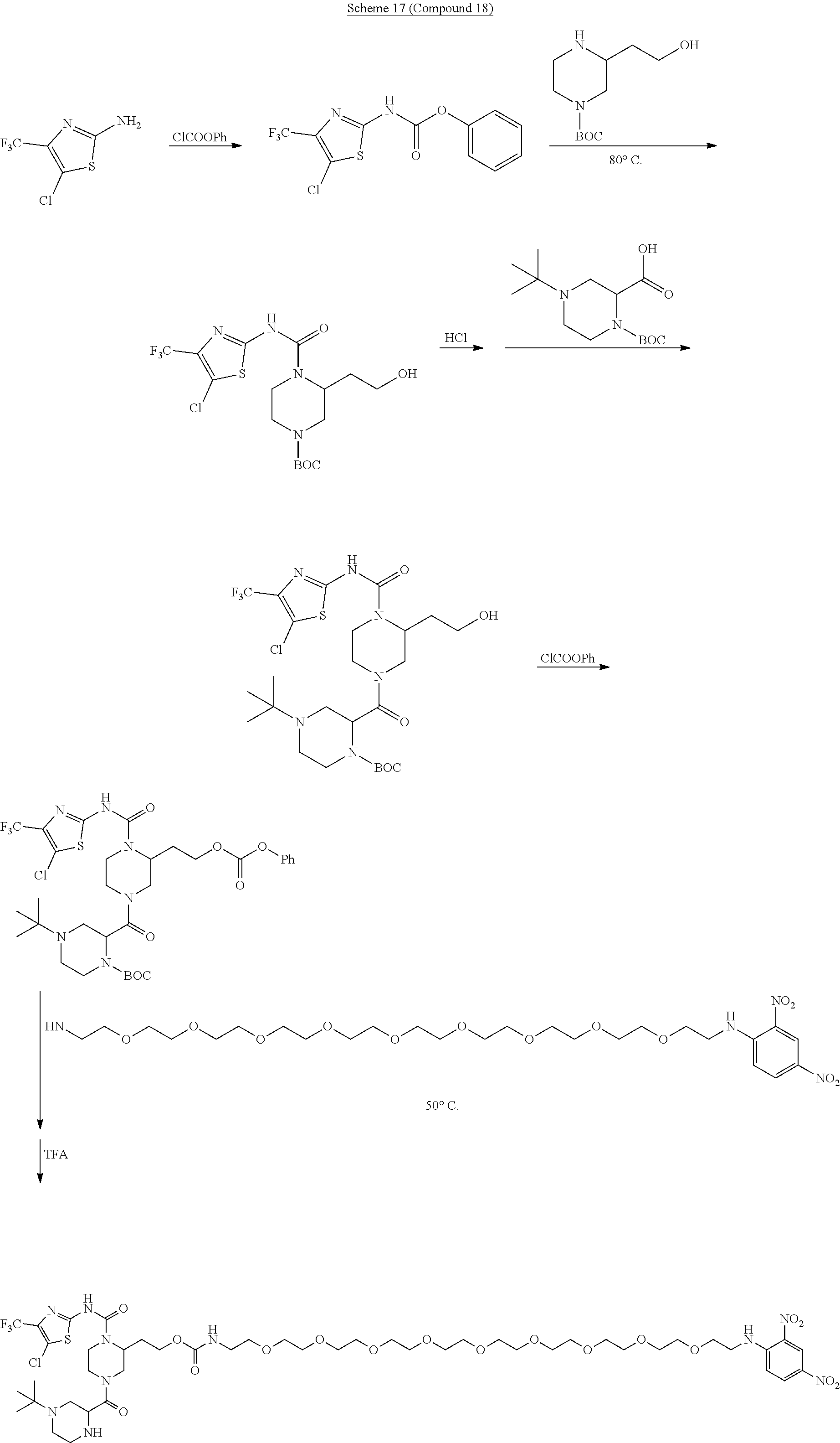

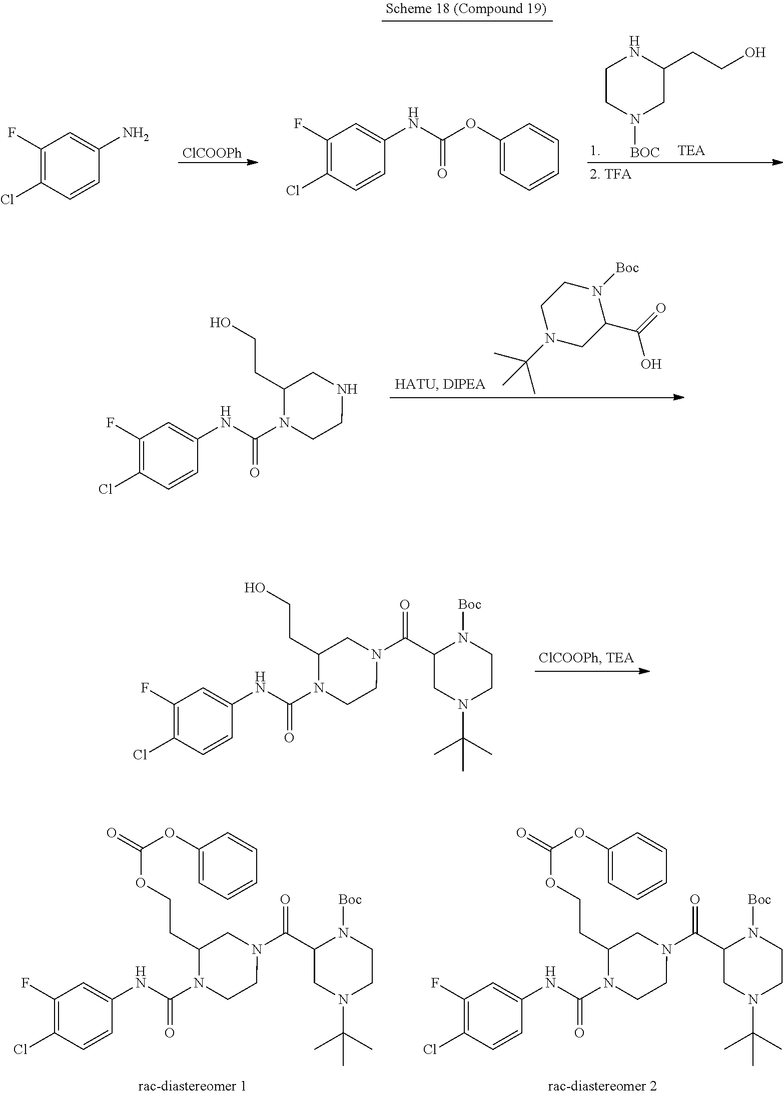

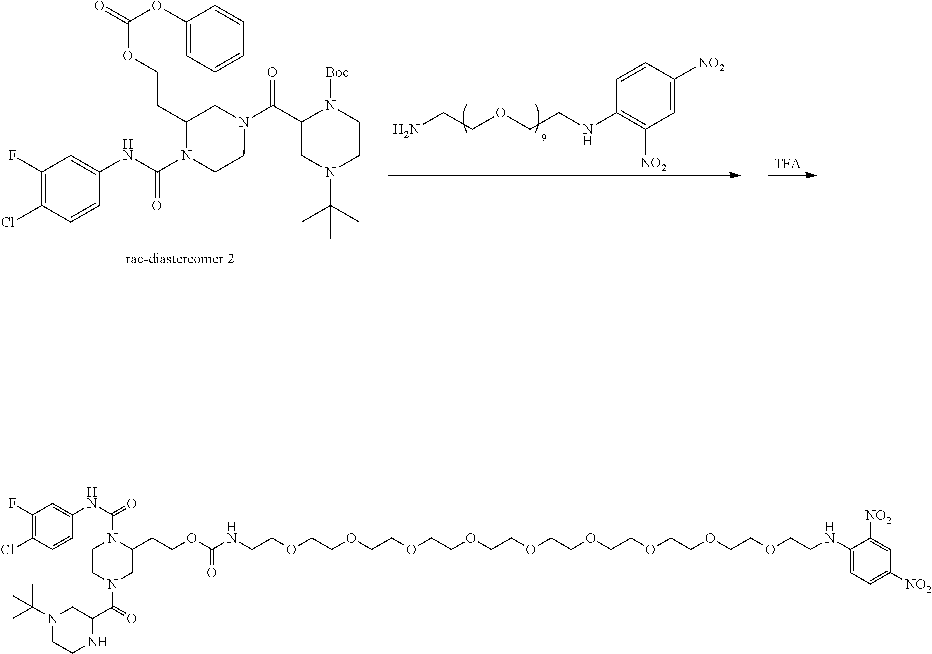

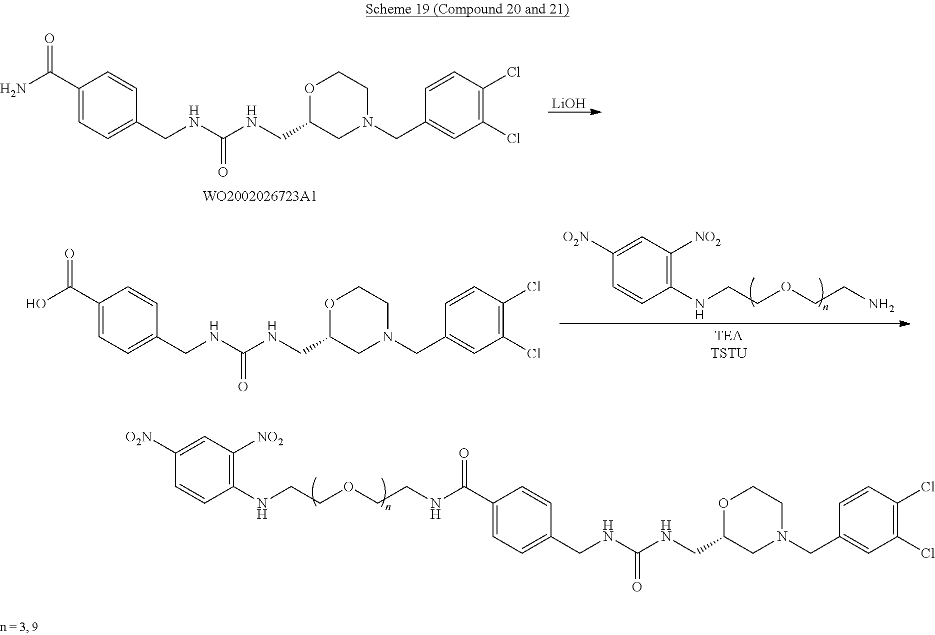

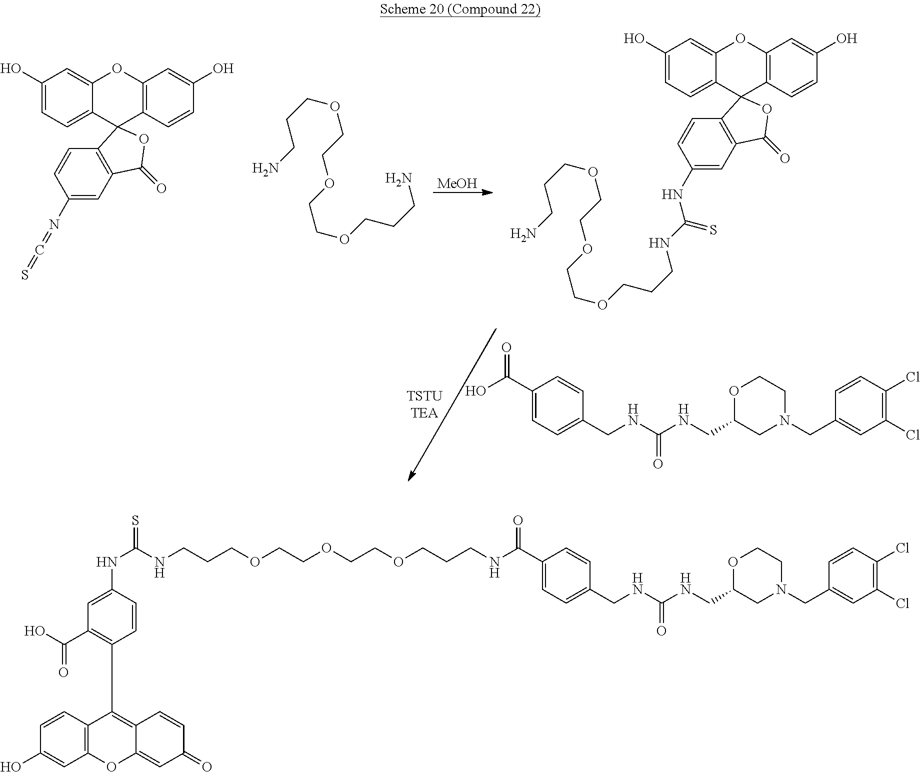 [SMILES: "MeOH" ; O=C1OC2(c3ccc(O)cc3Oc3cc(O)ccc32)c2ccc(N=C=S)cc21 ; NCCCOCCOCCOCCCN ; NCCCOCCOCCOCCCNC(=S)Nc1ccc2c(c1)C(=O)OC21c2ccc(O)cc2Oc2cc(O)ccc21 ; O=C(NCc1ccc(C(=O)O)cc1)NC[C@H]1CN(Cc2ccc(Cl)c(Cl)c2)CCO1 ; O=C(NCc1ccc(C(=O)NCCCOCCOCCOCCCNC(=S)Nc2ccc(-c3c4ccc(=O)cc-4oc4cc(O)ccc34)c(C(=O)O)c2)cc1)NC[C@H]1CN(Cc2ccc(Cl)c(Cl)c2)CCO1]

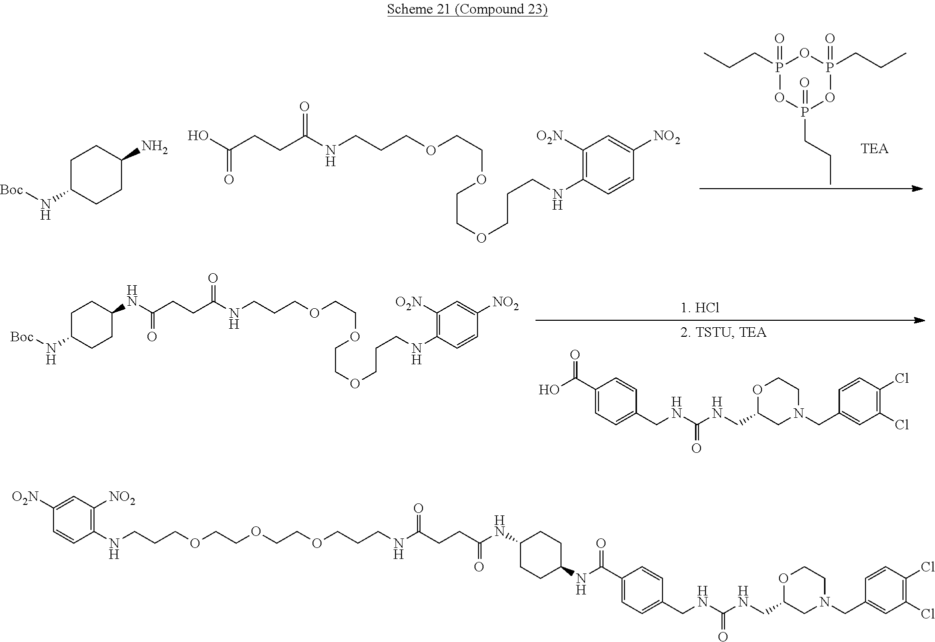

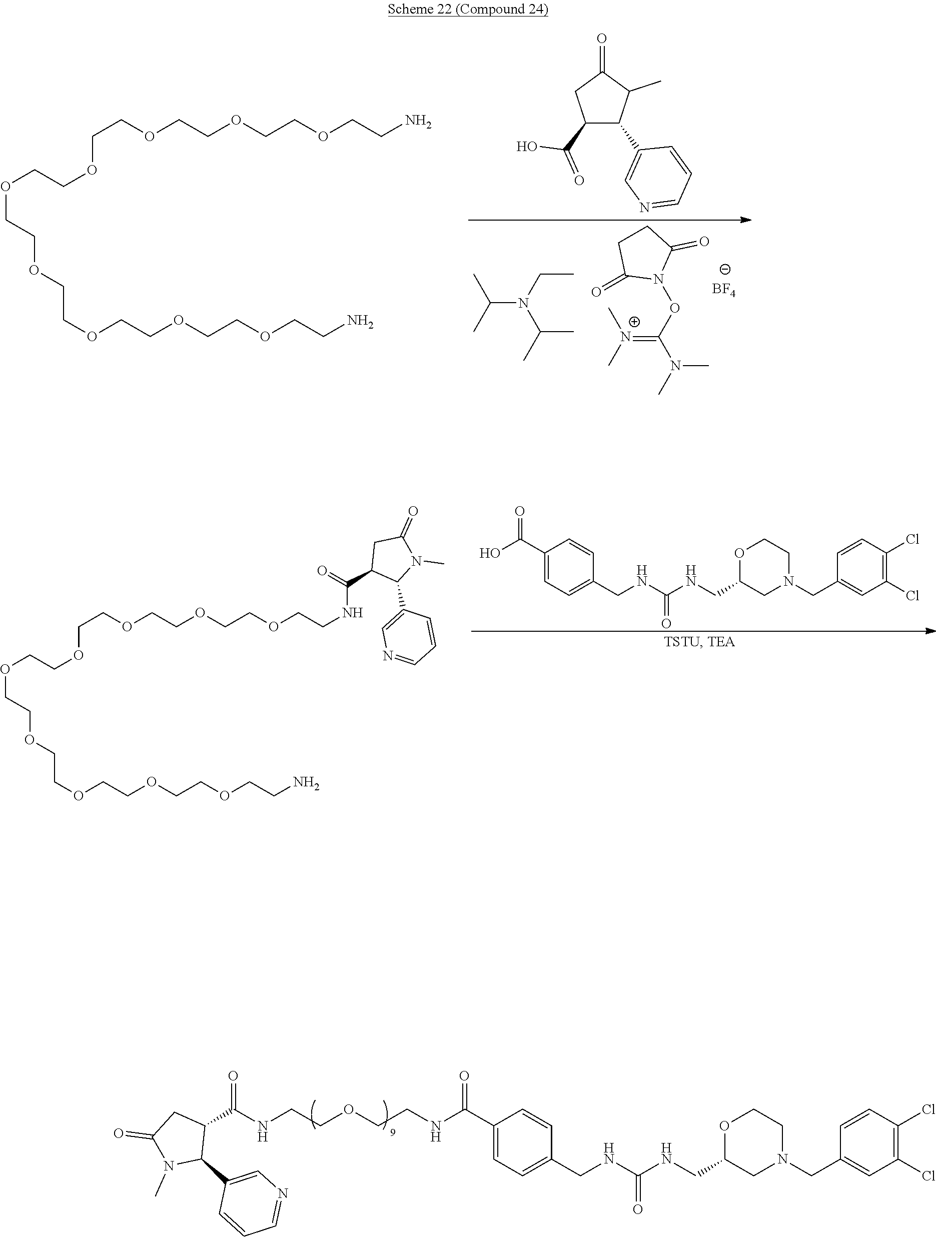

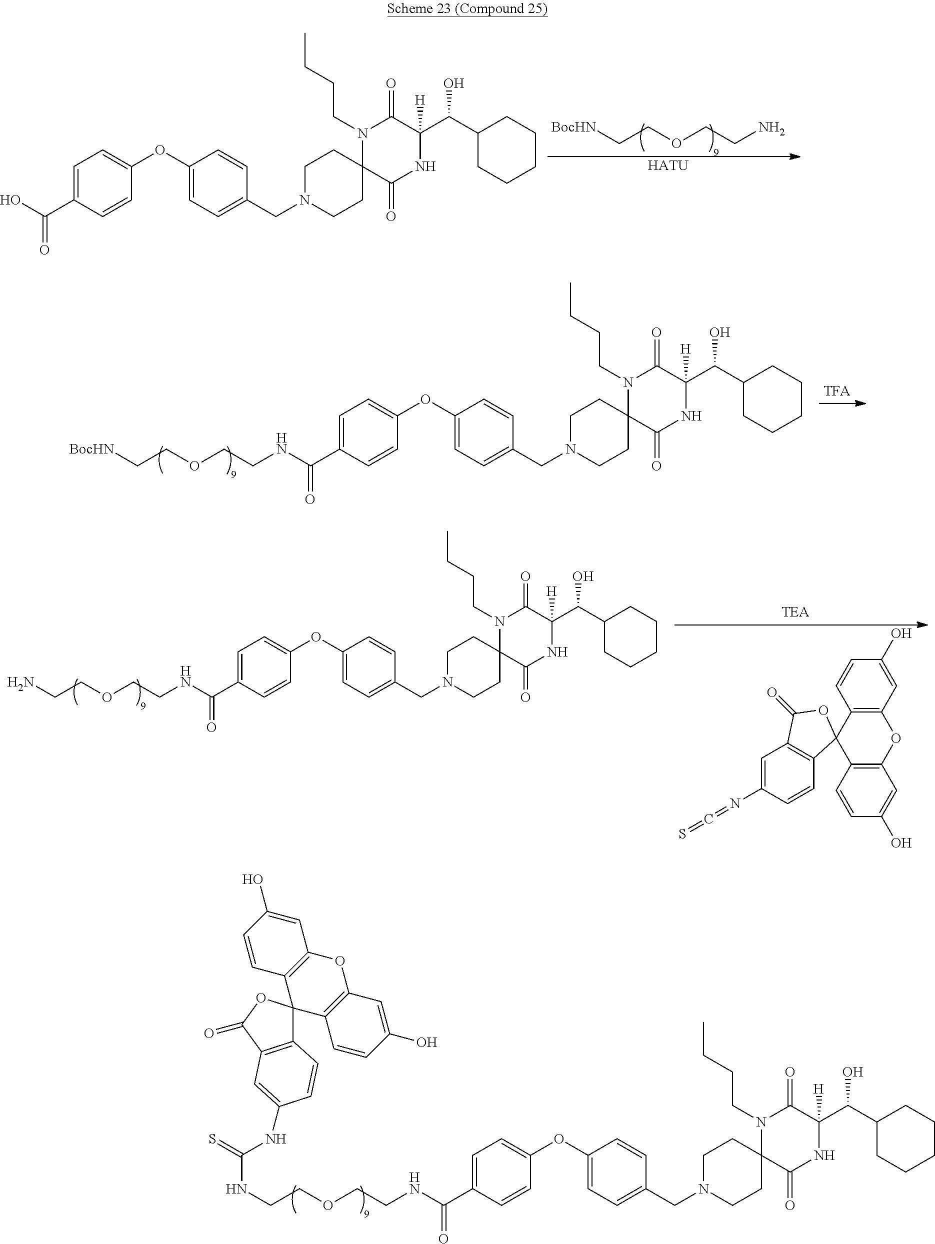

Section 1.2: Antibody Dependent Cellular Cytotoxicity Reporter Assay

This assay has four components
1) Heterobivalent Compound consisting of a targeting ligand and a hapten connected by a linker (ranging from 10 uM-1 pM)
2) A hapten antibody with functional Fc domain (typical concentrations are 0.01 ug/mL-200 ug/mL)
3) Target cells—CHOK1 cells engineered to overexpress either CCR1, CCR2, or CCR3, or CEMNKR cells engineered to overexpress CCR5 (typically 1000-20,000 cells per well)
4) Reporter cells—Jurkat cells engineered to express FcgRIIIa (ADCC reporter assay) and with a reporter gene (luciferase) under the control of the NFAT promoter (typically 3000-75,000 cells per well)

Reagents are combined in final volume of 20 uL in 384-well tissue culture treated plated.

All components are incubated together for ˜12-18 hours. Add BioGlo Detection reagent (from Promega) to lyse the cells and provide substrate for the luciferase reporter protein. Signal is measured on a microplate reader capable of measuring luminescence. Signal:Background is calculated by dividing the signal of a test well by the signal obtained when no compound is included in the assay. EC50 calculations were done using Graphpad Prism Software, specifically a nonlinear regression curve fit (Y=Bottom+(Top−Bottom)/(1+10\hat{ }((Log EC50−X)*HillSlope)))

For the purposes of administration, in certain embodiments, the HBM described herein are administered as a raw chemical or are formulated as pharmaceutical compositions. Pharmaceutical compositions disclosed herein include a HBM and one or more of: a pharmaceutically acceptable carrier, diluent or excipient. An HBM is present in the composition in an amount which is effective to treat a particular disease or condition of interest. The activity of HBM can be determined by one skilled in the art, for example, as described in the biological assays described below. Appropriate concentrations and dosages can be readily determined by one skilled in the art. In certain embodiments, HBM is present in the pharmaceutical composition in an amount from about 25 mg to about 500 mg. In certain embodiments, HMB is present in the pharmaceutical composition in an amount of about 100 mg to about 300 mg. In certain embodiments, HMB is present in the pharmaceutical composition in an amount of about 25 mg, 50 mg, 100 mg, 200 mg, 300 mg, 400 mg or about 500 mg, even higher.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

1. A method of destroying CCR-positive cells in a human, the method comprising administering to the human a Heterobivalent Molecule (HBM), wherein the HBM comprises a moiety binding to a CCR receptor on the cell and a moiety binding to endogenous or exogenous antibodies.

2. The method of claim 1 wherein the HBM further comprises a linker.

3. The method of claim 1 in which the cell destruction is mediated through ADCC, ADCP and/or CDC.

4. The method of claim 1 in which the CCR-positive cells being destroyed are cancer cells and/or pathogenic immune cells.

5. The method of claim 1 in which the CCR-positive cells being destroyed are pathogenic immune cells.

6. The method of claim 4 in which the cancer cells and/or pathogenic immune cells express one or more CCR receptors selected from the group of CCR1, CCR2, CCR3, and CCR5.

7. The method of claim 6 in which the CCR receptor is CCR1.

8. The method of claim 6 in which the CCR receptor is CCR2.

9. The method of claim 6 in which the CCR receptor is CCR3.

10. The method of claim 6 in which the CCR receptor is CCR5.

11. The method of claim 1 in which the moiety binding to endogenous or exogenous antibody is selected from the group consisting of DNP, fluorescein, cotinine and biotin.

12. A method of treating or preventing cancers, inflammatory disease, autoimmune disease, or allergic disease in a human patient comprising administering to the human patient a therapeutically effective amount of a Heterobivalent Molecule (HBM), wherein the HBM comprises a moiety binding to a CCR receptor on a cell and a moiety binding to endogenous or exogenous antibodies.

13. The method of claim 1 in which the HBM is administered orally.

14. A pharmaceutical composition comprising a Heterobivalent Molecule (HBM) and one or more pharmaceutically acceptable excipients, carriers, and/or diluents, wherein the HBM comprises a moiety binding to a CCR receptor on a cell and a moiety binding to endogenous or exogenous antibodies.
